(19) United States
(12) Patent Application Publication
(10) Pub. No.: US 2003/0166004 A1
(43) Pub. Date: Sep. 4, 2003

(54) Endothelial-cell binding peptides for diagnosis and therapy

(76) Inventors: Jeno Gyuris, Winchester, MA (US); Lou Lamphere, Newton, MA (US); Aaron J. Morris, Brighton, MA (US); Katherine Tsaioun, Belmont, MA (US)

Correspondence Address: ROPES & GRAY, ONE INTERNATIONAL PLACE, BOSTON, MA, 02110-2624, US

(21) Appl. No.: 10/286,457

(22) Filed: Nov. 1, 2002

Related U.S. Application Data
(60) Provisional application No. 60/334,822, filed on Nov. 1, 2001.

(51) Int. Cl. A61K 51/00; G06F 17/60; G01N 33/53; C07H 21/04; A61K 38/17; A61K 48/00; C12P 21/02; C12N 5/06

(52) U.S. Cl. 435/7.1; 435/69.1; 435/320.1; 435/226; 435/325; 514/44; 514/12; 424/1.11; 536/23.2; 705/2

(57) ABSTRACT

The present invention relates to peptides and their derivatives which bind to endothelial cells and inhibit their proliferation in in vitro assays, e.g., also referred to herein as endothelial cell binding peptide (ECBP) or ECBP sequence. These compositions may be combined with a pharmaceutically acceptable excipient or carrier and used to inhibit angiogenesis and angiogenesis-related diseases such as cancer, arthritis, macular degeneration, and diabetic retinopathy.

### REFERENCE TO RELATED APPLICATION

This application claims priority to U.S. Provisional Application No. 60/334,822, filed on Nov. 1, 2001, the entire contents of which are incorporated herein by reference.

### BACKGROUND OF THE INVENTION

Angiogenesis, the process by which new blood vessels are formed, is essential for normal body activities including reproduction, development and wound repair. Although the process is not completely understood, it is believed to involve a complex interplay of molecules which regulate the growth of endothelial cells (the primary cells of capillary blood vessels). Under normal conditions, these molecules appear to maintain the microvasculature in a quiescent state (i.e. one of no capillary growth) for prolonged periods which may last for as long as weeks or, in some cases, decades. When necessary (such as during wound repair), these same cells can undergo rapid proliferation and turnover within a 5 day period (Folkman, J. and Shing, Y., The Journal of Biological Chemistry, 267(16), 10931-10934, and Folkman, J. and Klagsbrun, M., Science, 235, 442-447 (1987).

Although angiogenesis is a highly regulated process under normal conditions, many diseases (characterized as angiogenic diseases) are driven by persistent unregulated angiogenesis. Otherwise stated, unregulated angiogenesis may either cause a particular disease directly or exacerbate an existing pathological condition. For example, ocular neovascularization has been implicated as the most common cause of blindness and dominates approximately 20 eye diseases. In certain existing conditions, such as arthritis, newly formed capillary blood vessels invade the joints and destroy cartilage. In diabetes, new capillaries formed in the retina invade the vitreous, bleed, and cause blindness. Growth and metastasis of solid tumors are also dependent on angiogenesis (Folkman, J., Cancer Research, 46, 467-473 (1986), Folkman, J., Journal of the National Cancer Institute, 82, 4-6 (1989). It has been shown, for example, that tumors which enlarge to greater than 2 mm must obtain their own blood supply and do so by inducing the growth of new capillary blood vessels. Once these new blood vessels become embedded in the tumor, they provide a means for tumor cells to enter the circulation and metastasize to distant sites such as liver, lung or bone (Weidner, N., et al., The New England Journal of Medicine, 324(1), 1-8 (1991).

Angiogenesis and angiogenesis related diseases are closely affected by cellular proliferation. The inhibition of endothelial cell proliferation also results in an inhibition of angiogenesis.

It is object of the invention to provide peptides or their derivatives that can inhibit abnormal or undesirable cellular proliferation, especially the growth of blood vessels into tumors. The peptides or their derivatives should be able to overcome the activity of endogenous growth factors in premetastatic tumors and prevent the formation of the capillaries in the tumors thereby inhibiting the growth of the tumors. The peptides or their derivatives should also be able to modulate the formation of capillaries in other angiogenic processes, such as wound healing and reproduction. The peptides or their derivatives for inhibiting angiogenesis should preferably be non-toxic and produce few side effects. In addition, the peptides or their derivatives should be capable of being conjugated to other molecules for both radioactive, non-radioactive or other labeling procedures for purposes of diagnosis, therapy or imagining.

### SUMMARY OF THE INVENTION

One aspect of the present invention relates to peptide sequences, which may be provided in the form of peptides, fusion proteins or peptidomimetics, which selectively bind to endothelial cells relative to other cells, e.g., also referred to herein as endothelial cell-binding peptides (ECBP) or ECBP sequence.

In certain embodiments, the present invention provides isolated, synthetic or recombinant peptides or polypeptides which includes one or more ECBP sequences, each having an amino acid sequence independently represented in any of the following sequences:

| | |
|---|---|
| T-h-s-X-X-X-X-u-s-G-s-G-K or | (SEQ ID NO: 531) |
| h-p-X-X-Y-t-h-X-s-s or | (SEQ ID NO: 532) |
| S-X-X-u-M-s-V or | (SEQ ID NO: 533) |
| t-h-h-s-L-R-h-X-a-u or | (SEQ ID NO: 534) |
| s-s-G-h-X-s-X-a-a-h-p-s or | (SEQ ID NO: 535) |
| p-s-a-h-X-X-T-s-V-P-h or | (SEQ ID NO: 536) |
| L-X-N-p-s-p-p-t-G-t-t-t or | (SEQ ID NO: 537) |
| h-h-P-+-h-h-L-p-p-h-h-t or | (SEQ ID NO: 538) |
| s-s-l-h-s-h-s-s-X-p-p-A or | (SEQ ID NO: 539) |
| S-s-c-N-H-X-t-X-X-c-s or | (SEQ ID NO: 540) |
| s-t-h-H-X-X-X-t-X-h-s-L or | (SEQ ID NO: 541) |
| h-h-h-T-S-h-p-X-X-t-X-t-s-h or | (SEQ ID NO: 542) |
| h-X-h-X-S-h-s-h-p-L-p or | (SEQ ID NO: 543) |
| s-X-T-hX-p-X-H-s-oh-s or | (SEQ ID NO: 544) |
| t-o-s-T-S-h-h-s-s or | (SEQ ID NO: 545) |
| L-P-s-X-K-p-h-T-p-u-h-s or | (SEQ ID NO: 546) |
| h-S-P-t-Q-Q-p-hp-s-x-T-u or | (SEQ ID NO: 547) |
| t-W-K-s-P-S or | (SEQ ID NO: 548) |
| h-T-S-P-h-h or | (SEQ ID NO: 549) |
| u-h-p-S-t-h-T-h or | (SEQ ID NO: 550) |
| h-h-h-h-P-H-X-h-t-s or | (SEQ ID NO: 551) |
| A-X-X-p-X-X-o-p-P-h-h or | (SEQ ID NO: 552) |
| s-h-h-G-o-L-h-X-p-s-T-t-s-s or | (SEQ ID NO: 553) |
| h-p-X-H-h-X-X-p-S-o-X-t or | (SEQ ID NO: 554) |
| u-X-u-X-h-s-X-X-P-R-X-h or | (SEQ ID NO: 555) |
| h-h-X-R-P-X-X-M-P or | (SEQ ID NO: 556) |
| h-s-Q-T-s-T-X-h-X-h-h or | (SEQ ID NO: 557) |
| p-A-s-s-s-X-X-p-s-p-h-u or | (SEQ ID NO: 558) |
| s-S-h-t-M-K-P-S-P-p-P-L-s-A or | (SEQ ID NO: 559) |
| s-A-h-P-A-t-p-h-X-t-h-s-s or | (SEQ ID NO: 560) |
| T-p-s-Y-P-s-R-h-G-s-p-P-H-P-c or | (SEQ ID NO: 561) |
| s-X-P-s-h-G-P-h-A-a or | (SEQ ID NO: 562) |
| s-h-W-X-s-X-X-h-s or | (SEQ ID NO: 563) |
| s-T-G-s-s-s-P-X-S-t or | (SEQ ID NO: 564) |
| s-s-s or | (SEQ ID NO: 565) |
| s-T-X-H-s-s-p or | (SEQ ID NO: 566) |
| A-t-s-s-P-+-V-h-s-L-s or | (SEQ ID NO: 567) |
| s-.-h-P-P-t-t-L or | (SEQ ID NO: 568) |
| t-X-X-X-L-X-s-s-h-s-p-s or | (SEQ ID NO: 569) |
| L-P-X-X-X-h-L or | (SEQ ID NO: 570) |
| T-t-h-h-s-h-P-R-h-X-t or | (SEQ ID NO: 571) |
| L-X-X-s-X-X-X-O-p-q or | (SEQ ID NO: 572) |
| s-X-h-L-p-T-P-t-X-c-a-p-N or | (SEQ ID NO: 573) |
| h-t-h-X-s-P-P-X-h-X-X-s or | (SEQ ID NO: 574) |
| h-h-h-P-X-h-T-s-X-X-S-p or | (SEQ ID NO: 575) |
| t-s-s-X-Q-P-S-X-h-s-A-p or | (SEQ ID NO: 576) |
| E-t-X-s-L-X-R-h-L or | (SEQ ID NO: 577) |
| H-t-X-p-X-P-P-s-L-h-p-X-L or | (SEQ ID NO: 578) |
| p-h-s-X-s-l-l-P-X-p-h-X-F or | (SEQ ID NO: 579) |
| p-p-X-X-s-A-h-X-s-l-u-P-X-s or | (SEQ ID NO: 580) |
| H-h-s-X-S-P-p-h-s-W-s-X-L or | (SEQ ID NO: 581) |
| W-P-h-t-D-h-P or | (SEQ ID NO: 582) |
| s-p-T-t-A-P-l-s-X-s-s-h or | (SEQ ID NO: 583) |
| h-X-s-s-h-T-o-h-R-S or | (SEQ ID NO: 584) |
| t-h-X-h-s-o-t-X-A-P-A-t-P-h or | (SEQ ID NO: 585) |
| M-+-X-s-h-p-A-P-s-s-t-A-h-H or | (SEQ ID NO: 586) |
| u-p-t-p-p-X-X-h-T-h-s-s-h-X-s or | (SEQ ID NO: 587) |
| Y-Y-P-A-X-S-t-h-p-o-R-s or | (SEQ ID NO: 588) |
| t-T-t-T-h-L-a-u-X-t-p-t or | (SEQ ID NO: 589) |
| p-X-X-h-H-s-T-h-p-s-H-t-h or | (SEQ ID NO: 590) |
| s-N-X-X-X-X-T-X-s-t-p-s-X-h-p or | (SEQ ID NO: 591) |
| A-t-o-h-s-P-X-A-s-h-h or | (SEQ ID NO: 592) |
| h-H-s-X-p-h-N-X-X-s-T-X-s- ++10 -s or | (SEQ ID NO: 593) |
| Y-q-h-X-s-X-p-s-X-p or | (SEQ ID NO: 594) |
| p-s-h-s-+-F-N-X-s-X-p-P or | (SEQ ID NO: 595) |
| s-p-h-s-X-h-s-P-u-X-h or | (SEQ ID NO: 596) |
| T-X-t-N-u-X-X-s-X-M-+-t or | (SEQ ID NO: 597) |
| S-M-V-Y-G-X-p-X-s-X-A or | (SEQ ID NO: 5981) |
| o-s-X-X-h-p-X-X-+-S-h-P-P-R-h or | (SEQ ID NO: 599) |
| t-h-t-P-h-S-X-S-h-X-h-P or | (SEQ ID NO: 600) |
| u-s-X-l-X-h-X-s-p-s or | (SEQ ID NO: 601) |
| h-t-Q-S-h-l-+-s-h-h-h-h or | (SEQ ID NO: 602) |
| s-p-p-X-h-s-L-t-S-s-p-X-h-h-h-D or | (SEQ ID NO: 603) |
| S-l-n-x-h-X-X-X-X-X-d-s or | (SEQ ID NO: 604) |
| S-L-H-X-L-X-t-D-h-h-h or | (SEQ ID NO: 605) |
| h-X-h-h-D-+-R-t-A-X-h-h or | (SEQ ID NO: 606) |
| p-X-u-X-X-X-X-R-X-s or | (SEQ ID NO: 607) |
| E-t-M-a-h-S-X-L or | (SEQ ID NO: 608) |
| N-X-X-X-X-p-p-h-h-t or | (SEQ ID NO: 609) |
| s-X-X-a-p-S-T-h-p-A-p-A or | (SEQ ID NO: 610) |
| S-p-X-h-X-t-Q-R-X-h-p-h or | (SEQ ID NO: 611) |
| p-X-h-X-Q-X-X-X-A-X-h-P or | (SEQ ID NO: 612) |
| u-s-X-p-h-t-X-S-S-h-t-h or | (SEQ ID NO: 613) |
| s-h-+-s-p-S-X-X-X-s-l-s-Y-p or | (SEQ ID NO: 614) |
| s-M-s-s-h-h-p-S-s-X-s-s-R or | (SEQ ID NO: 615) |
| h-h-s-h-Q-S-X-X-X-X-h or | (SEQ ID NO: 616) |
| o-X-h-s-S-M-h-h-h-s or | (SEQ ID NO: 617) |
| h-s-V-h-u-S-s-X-X-T or | (SEQ ID NO: 618) |
| h-S-t-L-P-H-h-X-L-s or | (SEQ ID NO: 619) |
| H-u-L-P-h-T-h-p-s-A-h or | (SEQ ID NO: 620) |
| h-h-X-p-p-T-H-X-h-P-h-p-s or | (SEQ ID NO: 621) |
| t-s-h-p-T-s-t-h-h-s-A or | (SEQ ID NO: 622) |
| t-s-T-s-Q-h-h-h-X-p-t-h or | (SEQ ID NO: 623) |
| h-X-h-s-p-D-V-p-h-X-h-h or | (SEQ ID NO: 624) |
| h-h-h-D-s-p-p-s-X-s-t-s-X-t or | (SEQ ID NO: 625) |
| p-X-X-X-X-X-R-h-T-X-h or | (SEQ ID NO: 626) |
| s-N-t-h-o-D-s-u-R-h or | (SEQ ID NO: 627) |
| I-X-X-X-c-L-T-X-P-s-P-t or | (SEQ ID NO: 628) |
| u-s-p-s-t-h-Q-s-R-t-h or | (SEQ ID NO: 629) |
| T-p-p-c-X-h-s-X-s-Y-h-A or | (SEQ ID NO: 630) |
| P-p-H-h-R-X-h-S-s-t-X-h or | (SEQ ID NO: 631) |
| s-p-+-c-h-p-X-u-R-t-h-p or | (SEQ ID NO: 632) |
| H-X-a-p-+-s-X-a-Y-p-s-A or | (SEQ ID NO: 633) |

wherein

X represents any amino acid residue;

o represents an amino acid with an alcoholic. side chain, e.g., Ser or Thr;

l represents an amino acid with an aliphatic side chain, e.g., Ile or Leu or Val;

a represents an amino acid with an aromatic side chain, e.g., Phe or His or Trp or Tyr;

c represents an amino acid with a charged side chain, e.g., Glu or Asp or His or Lys or Arg;

h represents an amino acid with a hydrophobic side chain, e.g., Ala or Cys or Phe or Gly or His or Ile or Lys or Leu or Met or Arg or Thr or Val Try or Tyr;

− represents an amino acid with a negatively charged side chain, e.g., Glu or Asp;

p represents an amino acid with a polar side chain, e.g., Cys or Asp or Glu or His or Lys or Asn or Gln or Arg or Ser or Thr;

+ represents an amino acid with a positive side chain, e.g., His or Lys or Arg;

s represents an amino acid with a small side chain, e.g., Ala or Cys or Asp or Gly or Asn or Pro or Ser or Thr or Val;

u represents an amino acid with a tiny side chain, e.g., Ala or Gly or Ser; and

t represents an amino acid most likely situated at a turn, e.g., Ala or Cys or Asp or Glu or Gly or His or Lys or Asn or Gln or Arg or Ser or Thr.

In certain preferred embodiments, the present invention provides isolated or recombinant peptides and polypeptides which include one or more ECBP sequences, each having an amino acid sequence independently represented in any of the following sequences:

| | |
|---|---|
| A-D-Y-R-S-SV-G-G-G-K or | (SEQ ID NO: 634) |
| L-S-N-N-s-K-H or | (SEQ ID NO: 635) |
| G-P-H-L-M-L-Q-N-K-L-R or | (SEQ ID NO: 636) |
| S-S-S-D-N-H-X-u-Q-L-H-T or | (SEQ ID NO: 637) |
| s-u-R-H-Q-S-W-H-P-H-D or | (SEQ ID NO: 638) |
| h-S-P-t-Q-Q-R-h-H-N-S-T or | (SEQ ID NO: 639) |
| A-P-I-H-L-H-S-c-P-L-L-H or | (SEQ ID NO: 640) |
| H-o-X-T-K-P-L or | (SEQ ID NO: 641) |
| H-s-I-Y-P-R-p or | (SEQ ID NO: 642) |
| Q-P-h-P-T-S-I or | (SEQ ID NO: 643) |
| h-A-s-u-S-M-P-T-s-R-L-A or | (SEQ ID NO: 644) |
| Y-H-h-P-P-S-s-T-P-L-s-A or | (SEQ ID NO: 645) |
| s-s-s-s-M-K-P-S-P-X-P or | (SEQ ID NO: 646) |
| T-T-s-Y-P-A-R-W-G-A-H-P or | (SEQ ID NO: 647) |
| L-P-I-s-K-A-L or | (SEQ ID NO: 648) |
| A-h-L-T-G-P-R or | (SEQ ID NO: 649) |
| p-S-L-H-Q-R-L or | (SEQ ID NO: 650) |
| H-Q-I-T-Q-P-p-S-L-L-S-P or | (SEQ ID NO: 651) |
| A-I-P-X-V-P or | (SEQ ID NO: 652) |
| H-K-A-P-S-P-K-h-D-W-s-P or | (SEQ ID NO: 653) |
| E-T-p-A-P-L or | (SEQ ID NO: 654) |
| G-E-T-X-A-P-h or | (SEQ iD NO: 655) |
| M-K-S-s-I-P-A-P-s-G-G or | (SEQ ID NO: 656) |
| S-P-F-R-A-P-s or | (SEQ ID NO: 657) |
| Y-P-h-R-A-P-T-s-Q-A-h-H or | (SEQ ID NO: 658) |
| S-T-A-o-Y-T-R or | (SEQ ID NO: 659) |
| Y-Y-P-A-u-S-T-I-Q-S-R-P or | (SEQ ID NO: 660) |
| H-D-T-Y-s-s-H or | (SEQ ID NO: 661) |
| H-A-A-T-M-P or | (SEQ ID NO: 662) |
| S-R-F-N-X-D or | (SEQ ID NO: 663) |
| T-X-p-N-G-P-S or | (SEQ ID NO: 664) |
| G-X-T-P-S-h-A or | (SEQ ID NO: 665) |
| S-M-V-Y-G-N-p-L-P-S-A-L or | (SEQ ID NO: 666) |
| h-A-h-S-M-P-P or | (SEQ ID NO: 667) |
| T-E-Q-p-W-I-K-N-I-Y-A-R or | (SEQ ID NO: 668) |
| A-L-H-S-A-R or | (SEQ ID NO: 669) |
| h-L-H-S-D-R-A-L-M-I-D or | (SEQ ID NO: 670) |
| S-A-P-L-t-S or | (SEQ ID NO: 671) |
| H-S-S-T-h-R-A or | (SEQ ID NO: 672) |
| S-p-P-W-s-A-Q-R-E-L-S-V or | (SEQ ID NO: 673) |
| u-T-W-S-H-H-h-S-S-u-u-L or | (SEQ ID NO: 674) |
| G-W-S-S-Y-R or | (SEQ ID NO: 675) |
| A-M-s-P-R-p-H-S-s-P-S-V or | (SEQ ID NO: 676) |
| M-P-A-V-M-S-S-s-Q-V-P-R or | (SEQ ID NO: 677) |
| L-L-A-D-T-T-H-H-h-P-W-T or | (SEQ ID NO: 678) |
| K-N-L-N-T-T-u-M-Y-A-A-S or | (SEQ ID NO: 679) |
| I-L-A-X-D-L-T-X-X-G-P or | (SEQ ID NO: 680) |
| O-G-K-W-Q-P-R or | (SEQ ID NO: 681) |
| G-L-Q-u-R-H-I or | (SEQ ID NO: 682) |
| K-h-I-P-t-T-Y or | (SEQ ID NO: 683) |
| Q-S-H-Y-R-X-I-S-P-A-Q-V | (SEQ ID NO: 684) |

wherein

X represents any amino acid residue;

o represents an amino acid with an alcoholic side chain, e.g., Ser or Thr;

l represents an amino acid with an aliphatic side chain, e.g., Ile or Leu or Val;

a represents an amino acid with an aromatic side chain, e.g., Phe or His or Trp or Tyr;

c represents an amino acid with a charged side chain, e.g., Glu or Asp or His or Lys or Arg;

h represents an amino acid with a hydrophobic side chain, e.g., Ala or Cys or Phe or Gly or His or Ile or Lys or Leu or Met or Arg or Thr or Val Try or Tyr;

− represents an amino acid with a negatively charged side chain, e.g., Glu or Asp;

p represents an amino acid with a polar side chain, e.g., Cys or Asp or Glu or His or Lys or Asn or Gin or Arg or Ser or Thr;

+ represents an amino acid with a positive side chain, e.g., His or Lys or Arg;

s represents an amino acid with a small side chain, e.g., Ala or Cys or Asp or Gly or Asn or Pro or Ser or Thr or Val;

u represents an amino acid with a tiny side chain, e.g., Ala or Gly or Ser;

t represents an amino acid most likely situated at a turn, e.g., Ala or Cys or Asp or Glu or Gly or His or Lys or Asn or Gln or Arg or Ser or Thr.

In another preferred embodiment, the invention provides isolated, synthetic or recombinant peptides or polypeptides which includes one or more ECBP sequences, each having an amino acid sequence independently represented in any of SEQ ID Nos: 1-530.

The ECBP sequence can be provided as a peptide, e.g., having 7 or more residues, e.g., preferably 7-12 or more amino acid residues. The subject ECBP sequence can also be present as a monomeric sequence in a larger polypeptide, or can be present in multiple copies having the same or different amino acid sequences. Whether provided in the form of a peptide or in the context of a larger polypeptide, the subject ECBP sequence can be selected by criteria which include its binding constant to endothelial cells.

Moreover, the ECBP sequence is a modular component, and can be added at various positions to a chimeric protein with no more than routine experimentation.

Another aspect of the invention provides a recombinant polypeptide which includes one or more ECBP sequences.

Another aspect of the invention provides a peptide or peptidomimetic or fusion protein, e.g., wherein one or more backbone bonds is replaced or one or more sidechains of a naturally occurring amino acid are replaced with sterically and/or electronically similar functional groups. In one embodiment, the invention provides a peptidomimetic comprising a binding sequence corresponding to an ECBP sequence represented in any of the above-described sequences or SEQ ID Nos: 1-530, having one or more peptide bond replacements or non-naturally occurring amino acid sidechains, wherein the peptidomimetic binds to a endothelial cell in a manner dependent upon the presence of the ECBP sequence.

In certain embodiments, the peptide or peptidomimetic or fusion protein is formulated in a pharmaceutically acceptable excipient.

In one embodiment, the ECBP sequence of any of the above peptide/polypeptide or the peptidomimetic thereof mediates binding to endothelial cells with a Kd of 10⁻⁵ or less.

In one embodiment, any of the above peptide/polypeptide or the peptidomimetic thereof has an EC₅₀ of 10⁻⁴ M or less for promoting at least one of endothelial cell proliferation or endothelial cell migration.

In one embodiment, any of the above peptide/polypeptide or the peptidomimetic thereof has an ED₅₀ of 10⁻⁴ M or less for inhibiting at least one of endothelial cell proliferation or endothelial cell migration.

In a preferred embodiment, any of the above peptide/polypeptide or the peptidomimetic thereof has an ED₅₀ for killing endothelial cells at least one order of magnitude greater than the ED₅₀ for inhibiting endothelial cell proliferation or endothelial cell migration.

In one embodiment, any of the above peptide/polypeptide or the peptidomimetic thereof is covalently or non-covalently coupled to a cytotoxic agent or antiproliferative agent.

In a preferred embodiment, the agent is selected from: alkylating agents, enzyme inhibitors, proliferation inhibitors, lytic agents, DNA or RNA synthesis inhibitors, membrane permeability modifiers, DNA intercalators, metabolites, dichloroethylsulfide derivatives, protein production inhibitors, ribosome inhibitors, inducers of apoptosis, and neurotoxins.

In one embodiment, any of the above peptide/polypeptide or the peptidomimetic thereof is coupled to a cytotoxic agents selected from taxanes, such as paclitaxel (Taxol®) and docetaxel (Taxotere®); nitrogen mustards, such as mechlorethamine, cyclophosphamide, melphalan, uracil mustard and chlorambucil; ethylenimine derivatives, such as thiotepa; alkyl sulfonates, such as busulfan; nitrosoureas, such as carmustine, lomustine, semustine and streptozocin; triazenes, such as dacarbazine; folic acid analogs, such as methotrexate; pyrimidine analogs, such as fluorouracil, cytarabine and azaribine; purine analogs, such as mercaptopurine and thioguanine; vinca alkaloids, such as vinblastine and vincristine; antibiotics, such as dactinomycin, daunorubicin, doxorubicin, bleomycin, mithramycin and mitomycin; enzymes, such as L-asparaginase; platinum coordination complexes, such as cisplatin; substituted urea, such as hydroxyurea; methyl hydrazine derivatives, such as procarbazine; adrenocortical suppressants, such as mitotane; hormones and antagonists, such as adrenocortisteroids (prednisone), progestins (hydroxyprogesterone caproate, medroprogesterone acetate and megestrol acetate), estrogens (diethylstilbestrol and ethinyl estradiol), antiestrogens (tamoxifen), and androgens (testosterone propionate and fluoxymesterone).

In one embodiment, any of the above peptide/polypeptide or the peptidomimetic thereof is coupled to a protein synthesis inhibitor, such as puromycin, cycloheximide, or ribonuclease.

In one embodiment, any of the above peptide/polypeptide or the peptidomimetic thereof is coupled to a toxin selected from ricin toxin, Pseudomonas exotoxin (PE), diphtheria toxin (DT), Clostridium perfringens phospholipase C (PLC), bovine pancreatic ribonuclease (BPR), pokeweed antiviral protein (PAP), abrin, abrin A chain (abrin toxin), cobra venom factor (CVF), gelonin (GEL), saporin (SAP), modeccin, viscumin or volkensin.

In one embodiment, any of the above peptide/polypeptide or the peptidomimetic thereof is coupled to an enzyme which converts a prodrug to an active drug.

In one embodiment, any of the above peptide/polypeptide or the peptidomimetic thereof is coupled to an agent selected from: metals; metal chelators; lanthanides; lanthanide chelators; radiometals; radiometal chelators; positron-emitting nuclei; microbubbles (for ultrasound); liposomes; molecules microencapsulated in liposomes or nanosphere; monocrystalline iron oxide nanocompounds; magnetic resonance imaging contrast agents; light absorbing, reflecting and/or scattering agents; colloidal particles; fluorophores, such as near-infrared fluorophores.

In a preferred embodiment, the peptide/polypeptide or the peptidomimetic is coupled to a metal-chelating ligand, such as an NₓS_{y} chelate moiety.

In a preferred embodiment, the metal-chelating ligand chelates a radiometal or paramagnetic ion.

Another aspect of the invention relates to an imaging preparation comprising the peptide/polypeptide or the peptidomimetic described above, including a chelated metal selected from ³²P, ³³P, ⁴³K, ⁴⁷Sc, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/^{81M}Kr, ^{87M}Sr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, 191Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi. Preferred therapeutic radionuclides include ¹⁸⁸Re, ¹⁸⁶Re, ²⁰³Pb, ²¹²Pb, ²¹²Bi, ¹⁰⁹Pd, ⁶⁴Cu, ⁶⁷Cu, ⁹⁰Y, ¹²⁵I, ¹³¹I, ⁷⁷Br, ²¹¹At, ⁹⁷Ru, ¹⁰⁵Rh, ¹⁹⁸Au and ¹⁹⁹Ag, ¹⁶⁶Ho or ¹⁷⁷Lu.

In one embodiment, any of the above peptide/polypeptide or the peptidomimetic thereof is coupled with a radiosensitizing agent.

In one embodiment, any of the above peptide/polypeptide or the peptidomimetic thereof is coupled to a polymer or a functionalized polymer.

In one embodiment, any of the above peptide/polypeptide or the peptidomimetic thereof is cyclic. In a preferred embodiment, the cyclic portion is formed by one or more intramolecular covalent bonds between two or more amino acid residues of the peptide. In a most preferred embodiment, the intramolecular bond is selected from: backbone-to-backbone, sidechain-to-backbone or sidechain-to-sidechain bonds. In another most preferred embodiment, said intramolecular bond is an intramolecular disulfide bond. In yet another most preferred embodiment, said intramolecular bond is selected from: backbone-to-backbone, sidechain-to-backbone or sidechain-to-sidechain bond.

In one embodiment, any of the above peptide/polypeptide or the peptidomimetic thereof is formulated with a polyanionic or polycatonic binding agent.

In one embodiment, any of the above peptide/polypeptide or the peptidomimetic thereof is formulated in a pharmaceutically acceptable excipient.

In one embodiment, any of the above peptide/polypeptide is a chimeric serum protein. In a preferred embodiment, the ECBP sequence(s) are present at one or more internal sites in the amino acid sequence of the serum protein. In another preferred embodiment, the serum protein is selected from: albumin, α-globulins, β-globulins, γ-globulins, haptoglobin, transthyretin, collagen, α2 macroglobulin, β2 microglobulin, C Reactive Protein, apolipoproteins, lipoproteins, cathepsins amylase, antichymotrypsin, ferritin, α fetoprotein, elastin and fibronectin and coagulation factors including fibrinogen, fibrin, thrombin, ceruloplasmin, antiplasmin or antithrombin III.

In one embodiment, any of the above peptide/polypeptide is a chimeric viral coat protein.

Another aspect of the invention relates to a nucleic acid encoding any of the polypeptide described above (which includes one or more ECBP sequence).

In a preferred embodiment, the nucleic acid encodes a chimeric serum protein fused to any of the above-described ECBP peptides/polypeptides. In a most preferred embodiment, the coding sequence is flanked at each end by a coding sequence of an intein polypeptide to encode a fusion protein which, when expressed, undergoes intramolecular splicing to yield a cyclic peptide including said one or more ECBP sequences.

In a preferred embodiment, the nucleic acid encodes a polypeptide that is a chimeric viral coat protein.

In a related aspect, the invention provides a viral particle including one or more chimeric viral coat proteins described above. In a preferred embodiment, the viral particle is an adenoviral particle or an adeno-associated viral particle. In a most preferred embodiment, the ECBP sequence is provided as part of a fusion protein including the fiber knob protein.

A related aspect of the invention relates to a nucleic acid including a coding sequence for any of the avove-described peptides/polypeptides.

One aspect of the present invention provides a method of treating, e.g., lessening the severity or preventing the occurrence of a disorder including unwanted proliferation of endothelial cells, especially those occurring in tumor blood vessels. In general, the subject method comprises administering to an animal, e.g., mammal, preferably a human, either (i) an ECBP or peptidomimetic or a fusion protein containing the ECBP sequences, or (ii) a gene construct for expressing the ECBP or fusion protein. The ECBP, peptidomimetic or gene construct is formulated in the pharmaceutical preparation for delivery into infected cells of the animal. The ECBP moiety may be associated with a toxin, a radioactive nuclei, a chemotherapeutic agent or agent which would be toxic when delivered to an endothelial cell.

Thus, the invention provides a method for promoting the proliferation and/or migration of endothelial cells comprising treating the cells with an ECBP agonist in an amount sufficient to promote proliferation and/or migration of the treated cells.

The invention also provides a method for reducing the proliferation and/or migration of endothelial cells comprising treating the cells with an ECBP antagonist in an amount sufficient to reduce proliferation and/or migration of the treated cells.

Yet another aspect of the invention provides a method for reducing angiogenesis comprising treating an mammal with an ECBP antagonist in an amount sufficient to reduce angiogenesis at one or more sites in the treated mammal.

In one embodiment, the method is for treatment of prophylaxis or reducing the effects of a disorder selected from: hemangioma, solid tumors, leukemia, metastasis, telangiectasia, psoriasis, scleroderma, pyogenic granuloma, myocardial angiogenesis, plaque neovascularization, coronary collaterals, cerebral collaterals, arteriovenous malformations, ischemic limb angiogenesis, corneal diseases, rubeosis, neovascular glaucoma, diabetic retinopathy, retrolental fibroplasia, arthritis, diabetic neovascularization, macular degeneration, wound healing, peptic ulcer, Helicobacter related diseases, fractures, keloids, vasculogenesis, hematopoiesis, ovulation, menstruation, placentation, or cat scratch fever.

In one embodiment, the method is part of a treatment regimen for treatment of a solid tumor or carcinoma.

In one embodiment, the method is part of a treatment regimen for treatment or prophylaxis for an autoimmune disease.

In one embodiment, the method is part of a treatment regimen for treatment or prophylaxis for an ocular diseases selected from: diabetic retinopathy, retinopathy of prematurity, corneal graft rejection, retrolental fibroplasia, neovascular glaucoma, rubeosis, retinal neovascularization due to macular degeneration or hypoxia.

In one embodiment, the method is part of a treatment regimen for treatment or prophylaxis for psoriasis.

In one embodiment, the method is used in combination with radiotherapy and/or other chemotherapeutic treatments.

In one embodiment, the ECBP antagonist used in any of the above methods is any of the peptide/polypeptide or peptidomimetic as described above.

Still another aspect of the invention provides a method for promoting angiogenesis comprising treating an mammal with an ECBP agonist in an amount sufficient to promote angiogenesis at one or more sites in the treated mammal.

In one embodiment, the method is part of a treatment regimen for myocardial infarction.

In one embodiment, the method is part of a treatment regimen for repair of vascular damage after ischemia.

In one embodiment, the method is part of a treatment regimen to stimulate the growth of transplanted tissue or vascularized prosthetic devices.

In one embodiment, the method is part of a treatment regimen to stimulate wound healing.

In one embodiment, the method is part of a treatment regimen for vascular tissue repair during or after angioplasty.

In one embodiment, the ECBP agonist used in any of the above methods is any of the peptide/polypeptide or peptidomimetic as described above.

In one embodiment, the ECBP agonist or antagonist used in any of the above methods is delivered systemically.

In one embodiment, the ECBP agonist or antagonist used in any of the above methods is delivered by local injection.

Still another aspect of the invention provides a medicament for promoting angiogenesis comprising an ECBP agonist in an amount sufficient to promote angiogenesis at one or more sites in the treated mammal.

Still another aspect of the invention provides a method for manufacturing a medicament for promoting angiogenesis comprising admixing an ECBP agonist, in an amount sufficient to promote angiogenesis at one or more sites in a treated mammal, with a pharmaceutically acceptable excipient.

Still another aspect of the invention provides a medicament for reducing angiogenesis comprising an ECBP antagonist in an amount sufficient to reduce angiogenesis at one or more sites in the treated mammal.

Still another aspect of the invention provides a method for manufacturing a medicament for inhibiting angiogenesis comprising admixing an ECBP antagonist, in an amount sufficient to reduce angiogenesis at one or more sites in a treated mammal, with a pharmaceutically acceptable excipient.

Still another aspect of the invention provides a method of imaging endothelial cells comprises administering to an animal, e.g., a human, an ECBP or peptidomimetic or fusion protein, associated with an imaging agent.

Yet another aspect of the invention provides a method of enhancing the infectivity of endothelial cells by viral particles comprising administering to an animal, e.g., a mammal, preferably a human, a viral particle having an ECBP or peptidomimetic or fusion protein associated with one or more coat proteins, as for example, the fiber knob of adenoviral or adeno-associated viral particles.

Yet another aspect of the invention relates to a pharmaceutical preparation comprising a therapeutically effective amount of an ECBP or peptidomimetic or fusion protein, formulated in the pharmaceutical preparation for delivery into infected cells of an animal, e.g. a mammal and preferably a human. In preferred embodiments, the polypeptide is formulated as a liposome.

Yet another aspect of the invention relates to a method of conducting a drug discovery business comprising: (i) identifying, from a variegated library of peptides, members of the library bind to endothelial cells; (ii) from the members of the library identified in step (i), identifying peptides which inhibit or promote growth and/or migration of endothelial cells; (iii) conducting therapeutic profiling of an agent including the peptide identified in step (ii), or peptidomimetic thereof or a protein containing the peptide for efficacy and toxicity in mammals; and (iv) formulating a pharmaceutical preparation including one or more agents identified in step (iii) as having an acceptable therapeutic profile.

In one embodiment, the method further includes an additional step of establishing a distribution system for distributing the pharmaceutical preparation for sale, and may optionally include establishing a sales group for marketing the pharmaceutical preparation.

Yet another aspect of the invention relates to a method of conducting a drug discovery business comprising: (i) identifying, from a variegated library of peptides, members of the library bind to endothelial cells; (ii) from the members of the library identified in step (i), identifying peptides which inhibit or promote growth and/or migration of endothelial cells; (iii) conducting therapeutic profiling of an agent including the peptide identified in step (ii), or peptidomimetic thereof or a protein containing the peptide for efficacy and toxicity in mammals; and (iv) licensing, to a third party, the rights for further drug development of one or more agents identified in step (iii) as having an acceptable therapeutic profile.

Yet another aspect of the invention relates to a method of conducting a drug discovery business comprising: (i) identifying, from a variegated library of peptides, members of the library bind to endothelial cells; (ii) from the members of the library identified in step (i), identifying peptides which inhibit or promote growth and/or migration of endothelial cells; (iii) licensing, to a third party, the rights for further drug development based on one or more peptides identified in step (ii).

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No.: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology, (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### BRIEF DESCRIPTION OF THE DRAWINGS

FIG. 1: Table listing all peptides isolated from random peptide libraries possessing an endothelial cell binding activity. It shows the individual sequences, sequence ID (SEQ ID Nos: 1-530) and the frequency of the peptides in the total sequenced population.

FIG. 2: ECBP motifs for 10% and corresponding motif subspace descriptors for 50% distance tree cuts.

FIG. 3: Figures showing binding of several representative synthetic peptides to endothelial cells.

FIG. 4: Figure showing inhibition of proliferation of several endothelial cell lines by several representative synthetic ECBPs.

FIG. 5: Figures showing greatly improved inhibition of proliferation of several endothelial cell lines by several representative mouse serum albumin-ECBP (MSA-ECBP) fusion proteins at much lower effective doses.

FIG. 6: Table summarizing RasMol's classification of the common amino acids.

FIG. 7: Inhibition of microvessel formation by EC-Binding Peptides in the Aortic Ring Assay. Eighteen identified peptides were tested over a range of concentrations (125 to 1000 μM), ten of them were found to be effective as angiogenesis inhibitor. Shown in this figure a positive result (7-H10) and a negative result (2-F8).

FIG. 8: List of 18 tested polypeptides, and their effects on angiogenesis inhibition. “+” designates a positive inhibitory function, “−” indicates undetectable or minimal effect in angiogenesis inhibition.

FIG. 9: Stimulation of angiogenesis in vivo by FGF in the mouse Matrigel plug assay.

FIG. 10: Inhibition of angiogenesis in the mouse Matrigel assay by EC-Binding Peptides. “n” represents the number of experiments conducted for each peptide/condition. “p” is the p-value for statistical significance. Except for the first set (+ and − FGF control), all experimental sets contains FGF, with the presence or absence of a test peptide being the variable being tested.

FIG. 11: Inhibition of angiogenesis in the mouse Matrigel assay by EC-Binding Peptides. Data in table.

FIG. 12: Dose-dependent inhibition of angiogenesis by 1-H5 in the mouse Matrigel assay.

FIG. 13: List of names, SEQ ID NOs., and sequences of peptides/polypeptides used in the examples.

### DETAILED DESCRIPTION OF THE INVENTION

I. Overview

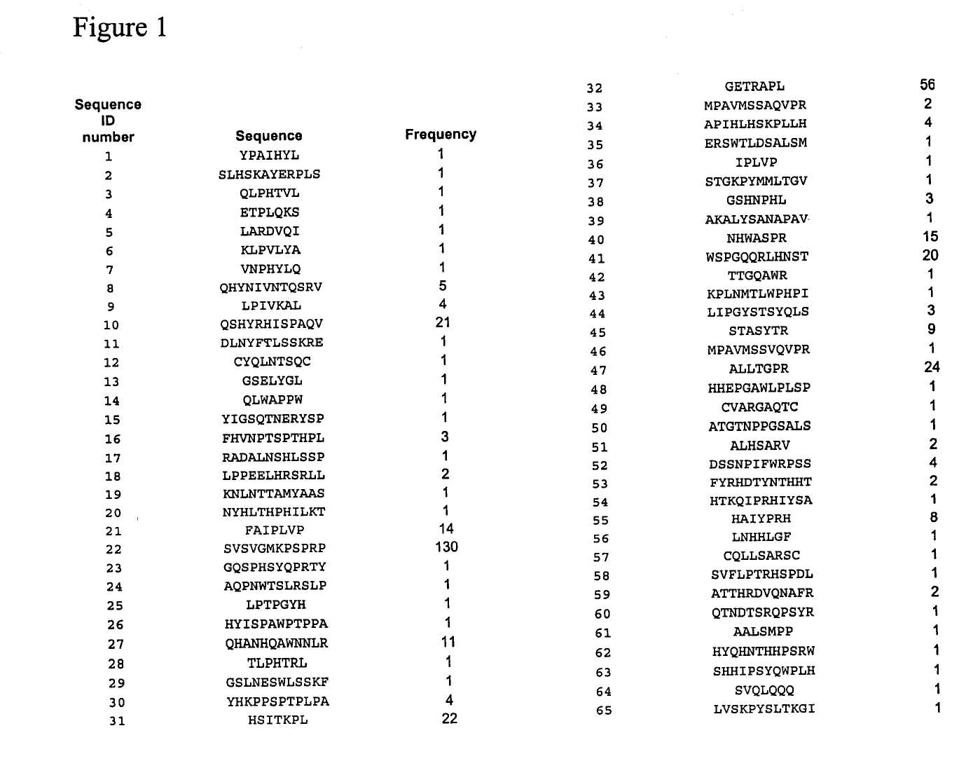

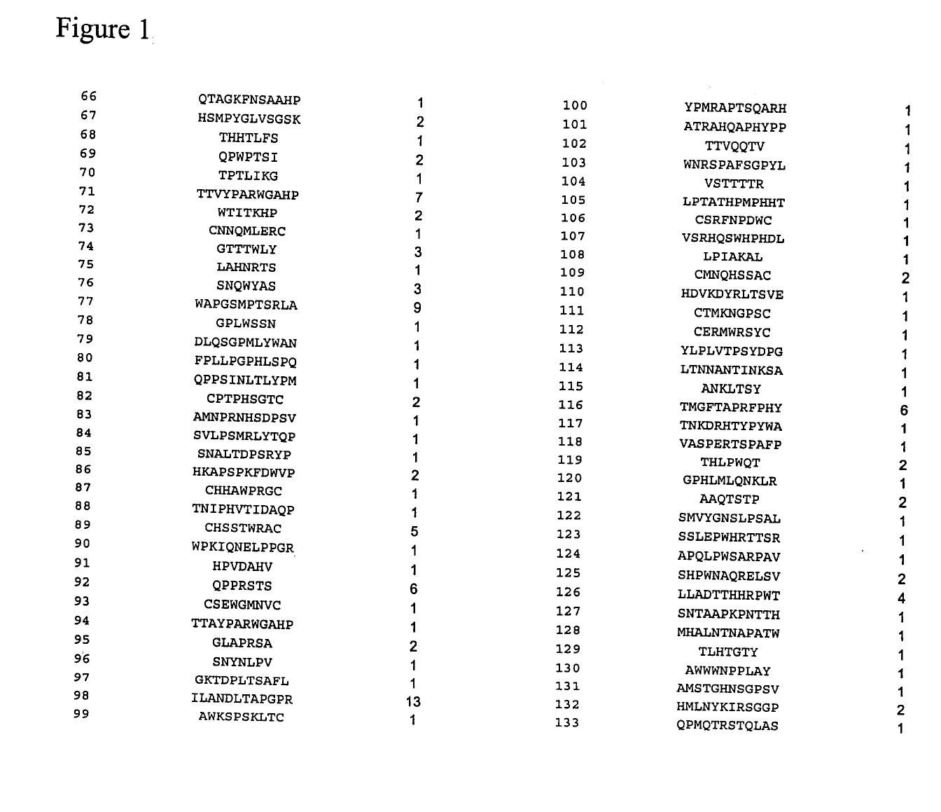

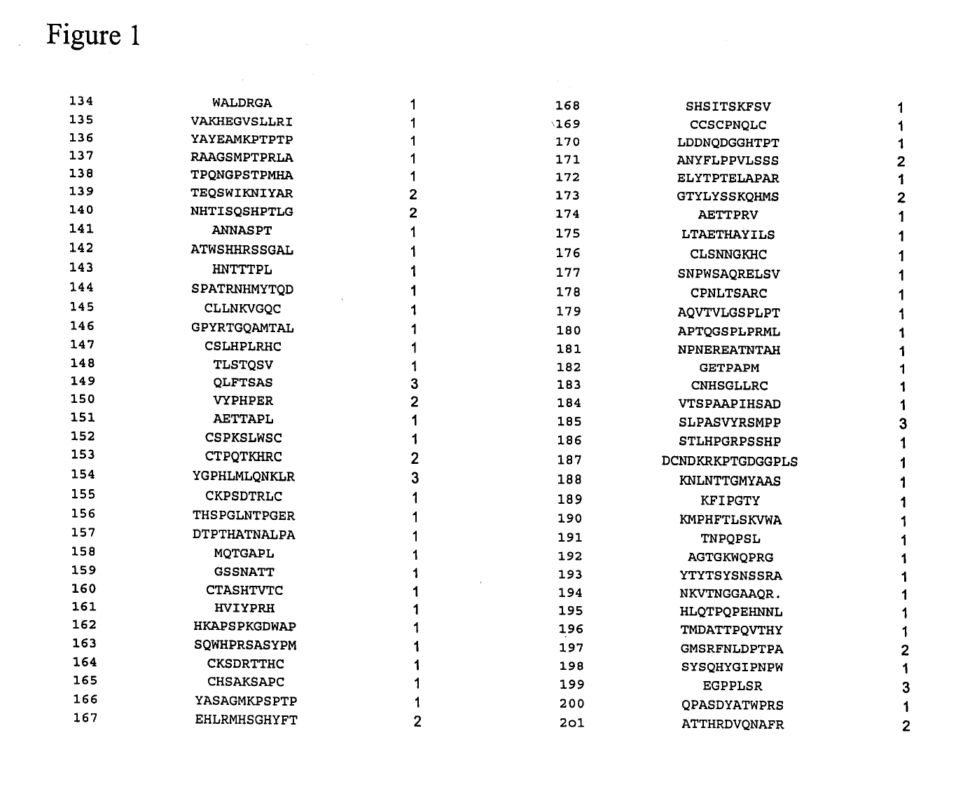

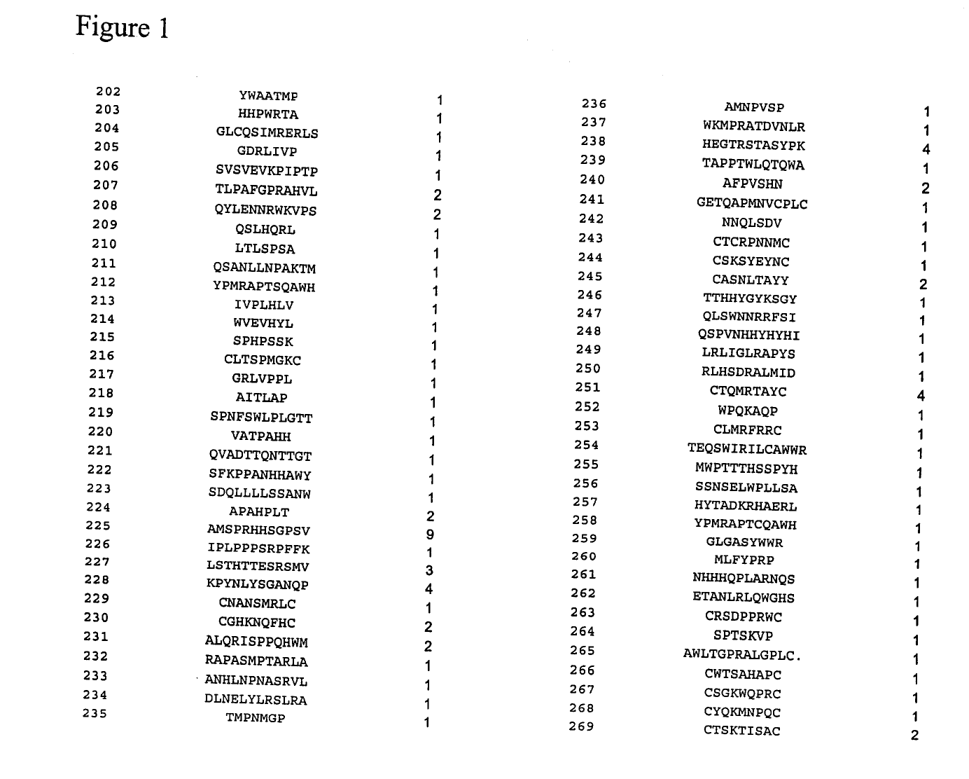

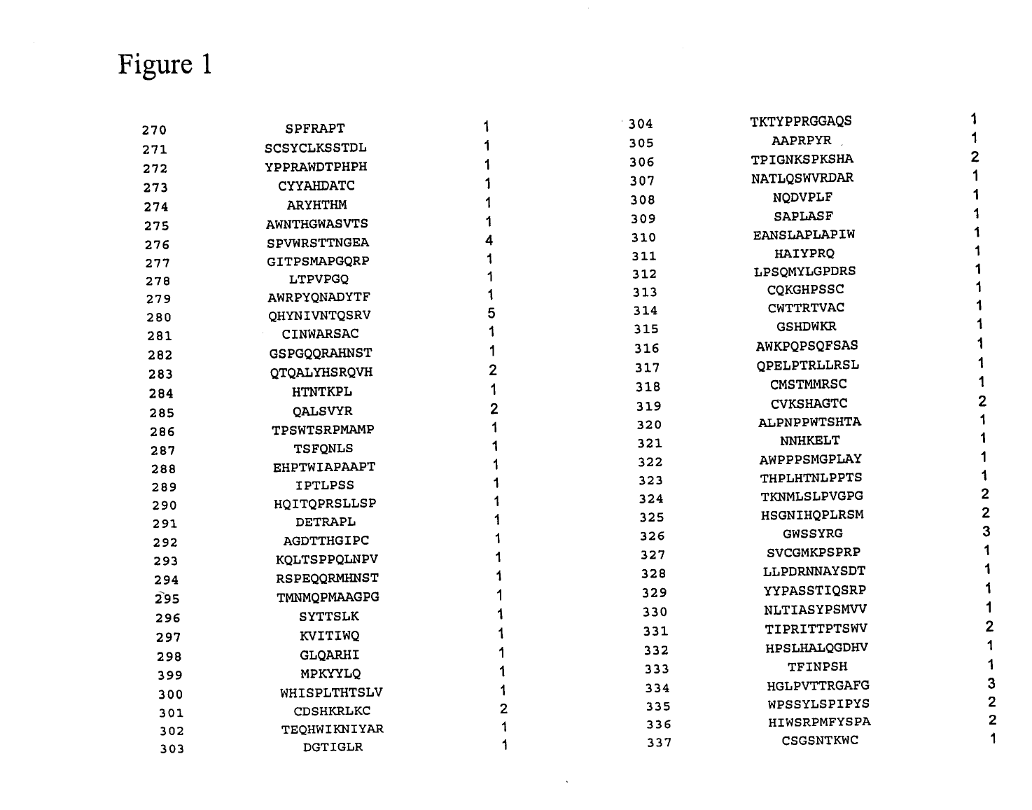

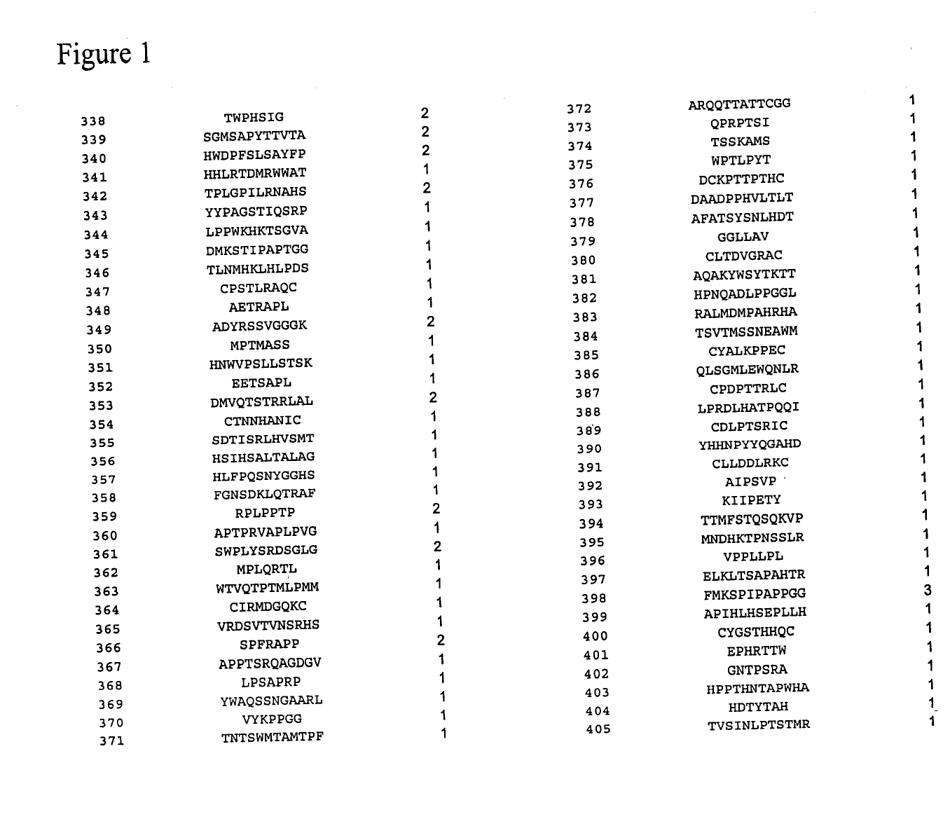

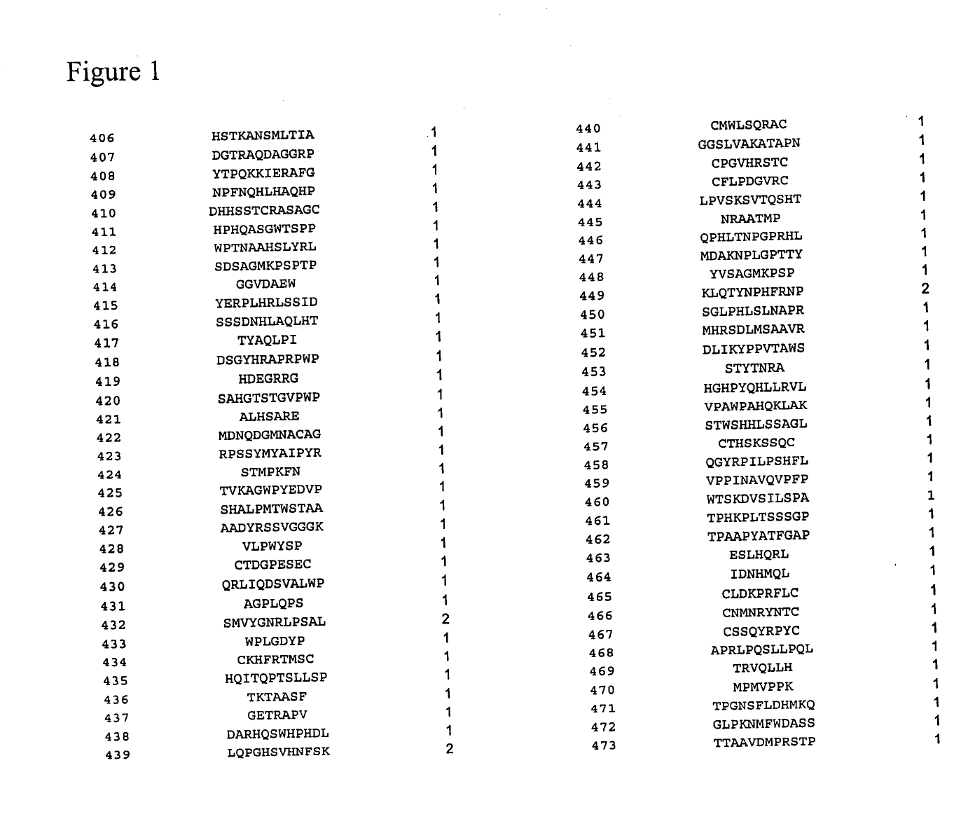

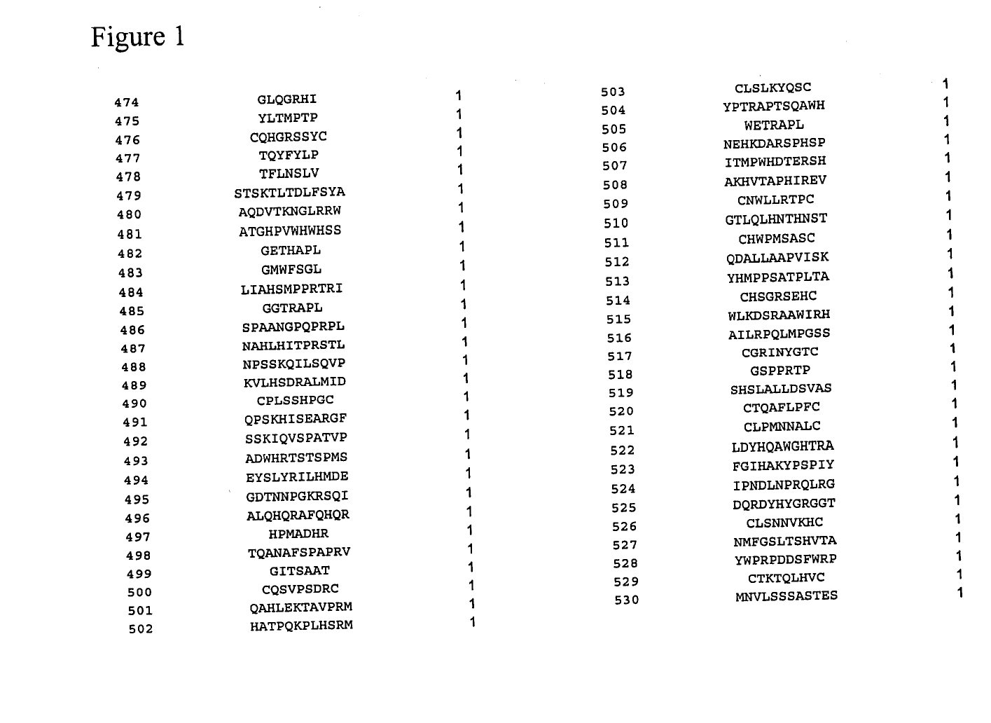

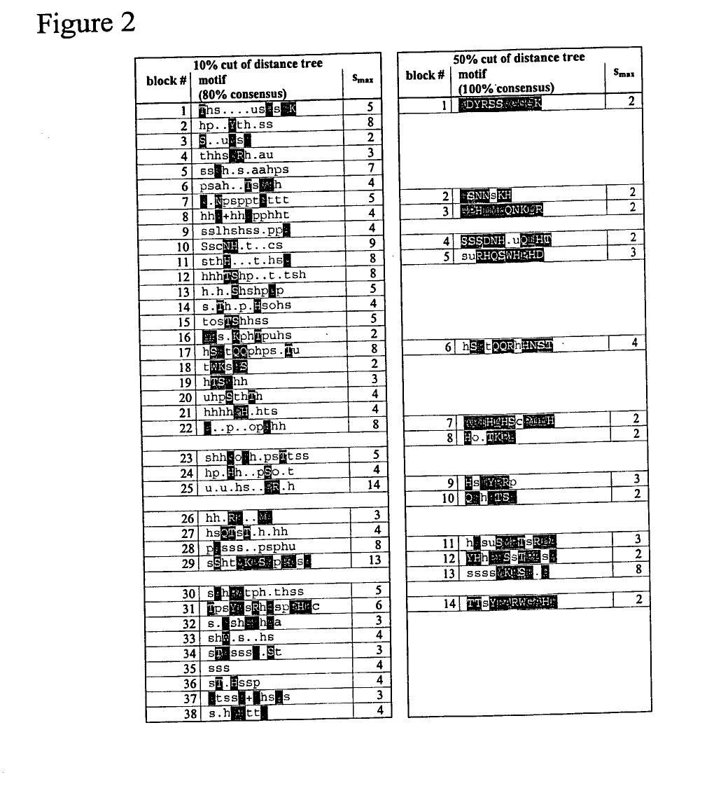

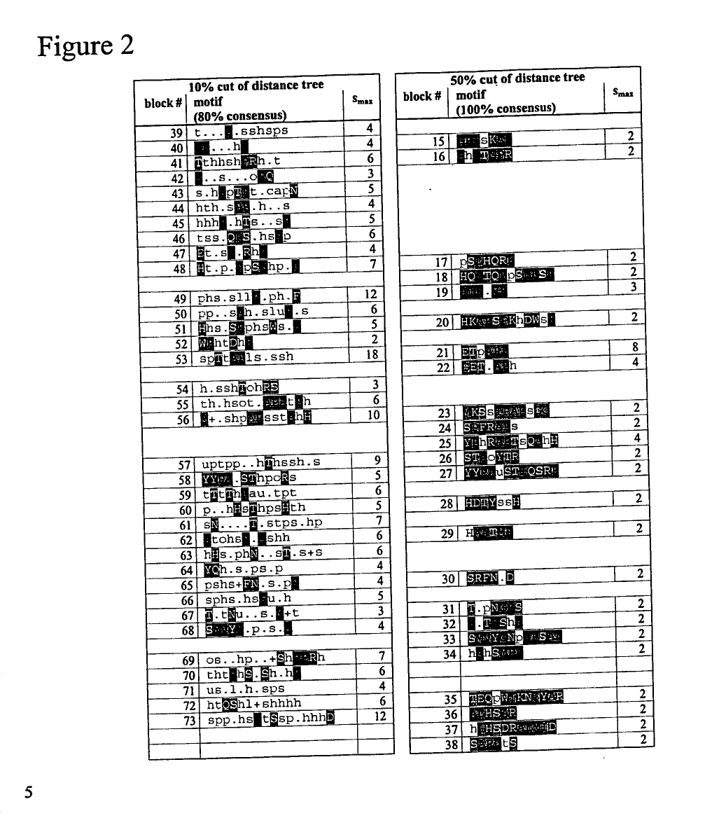

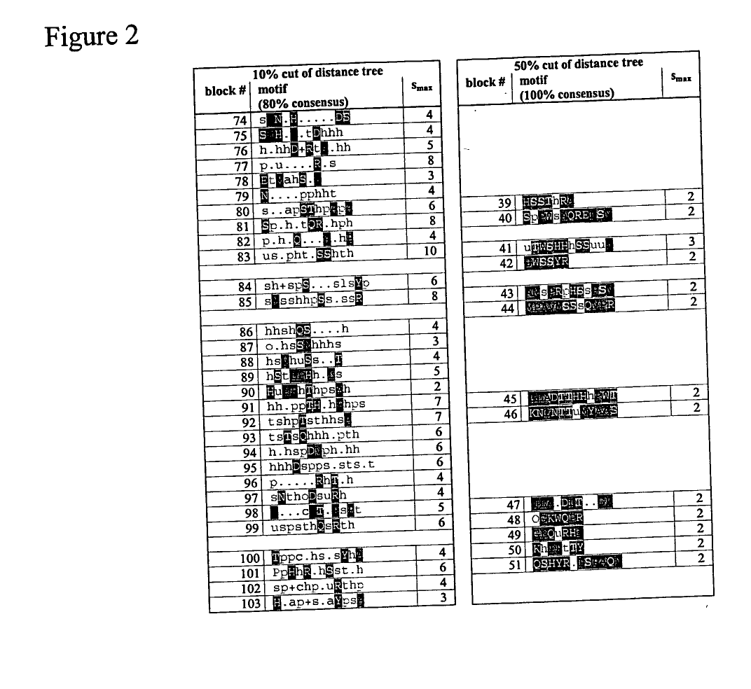

Three peptide libraries, containing linear 7 and 12-mer as well as cysteine constrained 7-mer peptides (C7C), were mixed and used in endothelial cell binding experiments to enrich for endothelial cell binding peptides (ECBP). After multiple rounds of affinity purification on bovine capillary endothelial (BCE) cells, the sequences of 1052 peptide were determined that correspond to 530 different peptides, as shown in FIG. 1.

II. Definitions

For convenience, certain terms employed in the specification, examples, and appended claims are collected here.

As used herein, the term “gene” or “recombinant gene” refers to a nucleic acid molecule comprising an open reading frame and including at least one exon and (optionally) an intron sequence. The term “intron” refers to a DNA sequence present in a given gene which is not translated into protein and is generally found between exons.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides.

The terms “protein”, “polypeptide” and “peptide” are used interchangeably herein when referring to a gene product, e.g., as may be encoded by a coding sequence.

In the phrase “isolated, synthetic or recombinant peptide or polypeptide”, the term “or” is used inclusively, e.g., certain peptides of the present invention may be both isolated and recombinantly produced.

As used herein, the term “transfection” means the introduction of a nucleic acid, e.g., an expression vector, into a recipient cell by nucleic acid-mediated gene transfer.

“Transcriptional regulatory sequence” is a generic term used throughout the specification to refer to DNA sequences, such as initiation signals, enhancers, and promoters, which induce or control transcription of protein coding sequences with which they are operably linked.

Operably linked is intended to mean that the nucleotide sequence is linked to a regulatory sequence in a manner which allows expression of the nucleotide sequence. Regulatory sequences are art-recognized and are selected to direct expression of the subject peptide. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990).

The term “gene construct” refers to a vector, plasmid, viral genome or the like which includes a coding sequence, can transfect cells, preferably mammalian cells, and can cause expression of the ECBP (or polypeptide including such moieties) or peptidomimetic of the cells transfected with the construct.

The term “interact” as used herein is meant to include detectable interactions between molecules, such as can be detected using, for example, a yeast two hybrid assay or by immunoprecipitation. The term interact is also meant to include “binding” interactions between molecules. Interactions may be, for example, protein-protein, protein-nucleic acid, protein-small molecule or small molecule-nucleic acid in nature. Preferred binding affinities have a Kd of 10⁻⁶ M or less, preferably ₁₀⁻⁸ or less, 10⁻⁹ or less, 10⁻¹⁰ or less, 10⁻¹¹ or less, or most preferably 10⁻¹² or less.

As used herein, the term “transfection” means the introduction of a nucleic acid, e.g., an expression vector, into a recipient cell by nucleic acid-mediated gene transfer. The term “transduction” is generally used herein when the transfection with a nucleic acid is by viral delivery of the nucleic acid. “Transformation”, as used herein, refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of a naturally-occurring form of the recombinant protein is disrupted.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The terms “chimeric”, “fusion” and “composite” are used to denote a protein, peptide domain or nucleotide sequence or molecule containing at least two component portions which are mutually heterologous in the sense that they are not, otherwise, found directly (covalently) linked in nature. More specifically, the component portions are not found in the same continuous polypeptide or gene in nature, at least not in the same order or orientation or with the same spacing present in the chimeric protein or composite domain. Such materials contain components derived from at least two different proteins or genes or from at least two non-adjacent portions of the same protein or gene. Composite proteins, and DNA sequences which encode them, are recombinant in the sense that they contain at least two constituent portions which are not otherwise found directly linked (covalently) together in nature.

An “ECBP”, which is used interchangeably herein with “ECBP sequence”, refers to an amino acid sequence that confers to a peptide or protein in which it is incorporated the ability to bind endothelial cells. In preferred embodiments, the subject ECBP sequence confers on a peptide or protein the ability to bind endothelial cells with an affinity constant (K_{d}) of 10⁻⁵ or less, and more preferably 10⁻⁶, 10⁻⁷, 10⁻⁸, 10⁻⁹ or less, or most preferably 10⁻¹⁰ or less.

The term “ECBP Therapeutic” as used herein is intended to generically encompass, unless otherwise obvious from its context, such molecules as polypeptides or peptides including an ECBP sequence, peptidomimetics and other small molecule mimics thereof, as well as expressions constructs of such peptides and polypeptides.

As used herein, the term “angiogenesis” means the generation of new blood vessels into a tissue or organ, and involves endothelial cell proliferation . Under normal physiological conditions, humans and other mammals undergo angiogenesis only in very specific restricted situations. For example, angiogenesis is normally observed in wound healing, fetal and embryonic development and formation of the corpus luteum, endometrium and placenta. The term “endothelium” means a thin layer of flat epithelial cells that lines serous cavities, lymph vessels, and blood vessels.

The term “ECBP therapeutic” refers to compositions which include an ECBP sequence, such as peptides and chimeric proteins, as well as the coding sequences for such peptides and proteins, and also to peptidomimetics of an ECBP sequence. The term also refers to compounds, such as small organic molecules, which compete an ECBP for endothelial cell binding and mimic the activity of the peptide, e.g., induce or inhibit endothelial cell proliferation.

As used herein, the term “antiangiogenesis activity” refers to the capability of a molecule to inhibit the growth of blood vessels.

As used herein, the term “endothelial inhibiting activity” refers to the capability of a molecule to inhibit angiogenesis in general and, for example, to inhibit the growth or migration of endothelial cells. In preferred embodiments, antagonist forms of the subject ECBP therapeutics have an IC₅₀ for endothelial inhibiting activity at least one order of magnitude, and more preferably at least two, three or even four orders of magnitude less than its IC₅₀ for killing endothelial cells.

An “antagonist” in the context of the present application refers to an ECBP therapeutic which, when bound to an endothelial cell, e.g., through a cell surface receptor, will inhibit EC proliferation, such as when assayed using the EC assay described below.

As used herein, the term “endothelial promoting activity” refers to the capability of a molecule to promote angiogenesis in general and, for example, to potentiate the growth or migration of endothelial cells. In preferred embodiments, agonist forms of the subject ECBP therapeutics have an EC₅₀ of 10⁻⁴M or less for promoting at least one of endothelial cell proliferation or endothelial cell migration, and more preferably 10⁻⁵M, 10⁻⁶M, 10⁻⁷M, or even 10⁻⁸M or less.

An “agonist” in the context of the present application refers to an ECBP therapeutic which, when bound to an endothelial cell, e.g., through a cell surface receptor, will promote EC proliferation, such as when assayed using the EC assay described below.

The “growth state” of a cell refers to the rate of proliferation of the cell and/or the state of differentiation of the cell. An “altered growth state” is a growth state characterized by an abnormal rate of proliferation, e.g., a cell exhibiting an increased or decreased rate of proliferation relative to a normal cell.

As used herein, “immortalized cells” refers to cells which have been altered via chemical and/or recombinant means such that the cells have the ability to grow through an indefinite number of divisions in culture.

A “patient” or “subject” to be treated by the subject method can mean either a human or non-human mammal.

As used herein, “proliferating” and “proliferation” refer to cells undergoing mitosis.

As used herein, “transformed cells” refers to cells which have spontaneously converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control.

As used herein, the term “prodrug” refers to compounds which are rapidly transformed in vivo to yield the parent compound, for example, by enzymatic hydrolysis in blood. A thorough discussion is provided in T. Higuchi and V. Stella, Prodrugs as Novel Delivery Systems, Vol. 14 of the A.C.S. Symposium Series and in Edward B. Roche, ed., Bioreversible Carriers in Drug Design, American Pharmaceutical Association and Permagon Press, 1987.

As used herein, the term “pharmaceutically acceptable prodrug” refers to (1) those prodrugs of the compounds of the present invention which are, within the scope of sound medical judgment, suitable for use in contact with the tissues of humans and other mammal without undue toxicity, irritation, allergic response and the like, commensurate with a suitable benefit-to-risk ratio and effective for their intended use and (2) zwitterionic forms, where possible, of the parent compound.

The term “amino acid residue” is known in the art. In general the abbreviations used herein for designating the amino acids and the protective groups are based on recommendations of the IUPAC-IUB Commission on Biochemical Nomenclature (see Biochemistry (1972) 11:1726-1732). In certain embodiments, the amino acids used in the application of this invention are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan.

The term “amino acid residue” further includes analogs, derivatives and congeners of any specific amino acid referred to herein, as well as C-terminal or N-terminal protected amino acid derivatives (e.g. modified with an N-terminal or C-terminal protecting group). For example, the present invention contemplates the use of amino acid analogs wherein a side chain is lengthened or shortened while still providing a carboxyl, amino or other reactive precursor functional group for cyclization, as well as amino acid analogs having variant side chains with appropriate functional groups). For instance, the subject compound can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminopimelic acid, ornithine, or diaminobutyric acid. Other naturally occurring amino acid metabolites or precursors having side chains which are suitable herein will be recognized by those skilled in the art and are included in the scope of the present invention.

As used herein, the term “N-protecting group” refers to those groups intended to protect the α-N-terminal of an amino acid or peptide or to otherwise protect the amino group of an amino acid or peptide against undesirable reactions during synthetic procedures. Commonly used N-protecting groups are disclosed in Greene, “Protective Groups In Organic Synthesis,” (John Wiley & Sons, New York (1981)), which is hereby incorporated by reference. Additionally, protecting groups can be used as prodrugs which are readily cleaved in vivo, for example, by enzymatic hydrolysis, to release the biologically active parent. N-protecting groups comprise loweralkanoyl groups such as formyl, acetyl (“Ac”), propionyl, pivaloyl, t-butylacetyl and the like; other acyl groups include 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl and the like; sulfonyl groups such as benzenesulfonyl, p-toluenesulfonyl and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxy-carbonyl, 2-nitro-4,5-dimethoxybenzyloxycarbonyl, 3,4,5-trimethoxybenzyloxycarbonyl, 1-(p-biphenylyl)-1-methylethoxycarbonyl, .alpha.,.alpha.-dimethyl-3,5-dimethoxybenzyloxycarbonyl, benzhydryloxycarbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl and the like; arylalkyl groups such as benzyl, triphenylmethyl, benzyloxymethyl, 9-fluorenylmethyloxycarbonyl (Fmoc) and the like and silyl groups such as trimethylsilyl and the like. Preferred N-protecting groups are formyl, acetyl, benzoyl, pivaloyl, t-butylacetyl, phenylsulfonyl, benzyl, t-butyloxycarbonyl (Boc) and benzyloxycarbonyl (Cbz). For example, lysine may be protected at the .alpha.-N-terminal by an acid labile group (e.g. Boc) and protected at the ε-N-terminal by a base labile group (e.g. Fmoc) then deprotected selectively during synthesis.

As used herein, the term “carboxy protecting group” refers to a carboxylic acid protecting ester or amide group employed to block or protect the carboxylic acid functionality while the reactions involving other functional sites of the compound are performed. Carboxy protecting groups are disclosed in Greene; “Protective Groups in Organic Synthesis” pp. 152-186 (1981), which is hereby incorporated by reference. Additionally, a carboxy protecting group can be used as a prodrug whereby the carboxy protecting group can be readily cleaved in vivo, for example by enzymatic hydrolysis, to release the biologically active parent. Such carboxy protecting groups are well known to those skilled in the art, having been extensively used in the protection of carboxyl groups in the penicillin and cephalosporin fields as described in U.S. Pat. Nos. 3,840,556 and 3,719,667, the disclosures of which are hereby incorporated herein by reference. Representative carboxy protecting groups are C₁-C₈ loweralkyl (e.g., methyl, ethyl or t-butyl and the like); arylalkyl such as phenethyl or benzyl and substituted derivatives thereof such as alkoxybenzyl or nitrobenzyl groups and the like; arylalkenyl such as phenylethenyl and the like; aryl and substituted derivatives thereof such as 5-indanyl and the like; dialkylaminoalkyl such as dimethylaminoethyl and the like); alkanoyloxyalkyl groups such as acetoxymethyl, butyryloxymethyl, valeryloxymethyl, isobutyryloxymethyl, isovaleryloxymethyl, 1-(propionyloxy)-1-ethyl, 1-(pivaloyloxyl)-1-ethyl, 1-methyl-1-(propionyloxy)-1-ethyl, pivaloyloxymethyl, propionyloxymethyl and the like; cycloalkanoyloxyalkyl groups such as cyclopropylcarbonyloxymethyl, cyclobutylcarbonyloxymethyl, cyclopentylcarbonyloxymethyl, cyclohexylcarbonyloxymethyl and the like; aroyloxyalkyl such as benzoyloxymethyl, benzoyloxyethyl and the like; arylalkylcarbonyloxyalkyl such as benzylcarbonyloxymethyl, 2-benzylcarbonyloxyethyl and the like; alkoxycarbonylalkyl or cycloalkyloxycarbonylalkyl such as methoxycarbonylmethyl, cyclohexyloxycarbonylmethyl, 1-methoxycarbonyl-1-ethyl and the like; alkoxycarbonyloxyalkyl or cycloalkyloxycarbonyloxyalkyl such as methoxycarbonyloxymethyl, t-butyloxycarbonyloxymethyl, 1-ethoxycarbonyloxy-1-ethyl, 1-cyclohexyloxycarbonyloxy-1-ethyl and the like; aryloxycarbonyloxyalkyl such as 2-(phenoxycarbonyloxy)ethyl, 2-(5-indanyloxycarbonyloxy)ethyl and the like; alkoxyalkylcarbonyloxyalkyl such as 2-(1-methoxy-2-methylpropan-2-oyloxy)ethyl and like; arylalkyloxycarbonyloxyalkyl such as 2-(benzyloxycarbonyloxy)ethyl and the like; arylalkenyloxycarbonyloxyalkyl such as 2-(3-phenylpropen-2-yloxycarbonyloxy)ethyl and the like; alkoxycarbonylaminoalkyl such as t-butyloxycarbonylaminomethyl and the like; alkylaminocarbonylaminoalkyl such as methylaminocarbonylaminomethyl and the like; alkanoylaminoalkyl such as acetylaminomethyl and the like; heterocycliccarbonyloxyalkyl such as 4-methylpiperazinylcarbonyloxymethyl and the like; dialkylaminocarbonylalkyl such as dimethylaminocarbonylmethyl, diethylaminocarbonylmethyl and the like; (5-(loweralkyl)-2-oxo-1,3-dioxolen-4-yl)alkyl such as (5-t-butyl-2-oxo-1,3-dioxolen-4-yl)methyl and the like; and (5-phenyl-2-oxo-1,3-dioxolen-4-yl)alkyl such as (5-phenyl-2-oxo-1,3-dioxolen-4-yl)methyl and the like.

Also included are the (D) and (L) stereoisomers of such amino acids when the structure of the amino acid admits of stereoisomeric forms. The configuration of the amino acids and amino acid residues herein are designated by the appropriate symbols (D), (L) or (DL), furthermore when the configuration is not designated the amino acid or residue can have the configuration (D), (L) or (DL). It will be noted that the structure of some of the compounds of this invention includes asymmetric carbon atoms. It is to be understood accordingly that the isomers arising from such asymmetry are included within the scope of this invention. Such isomers can be obtained in substantially pure form by classical separation techniques and by sterically controlled synthesis. For the purposes of this application, unless expressly noted to the contrary, a named amino acid shall be construed to include both the (D) or (L) stereoisomers. D- and L-α-Amino acids are represented by the following Fischer projections and wedge-and-dash drawings. In the majority of cases, D- and L-amino acids have R- and S-absolute configurations, respectively.

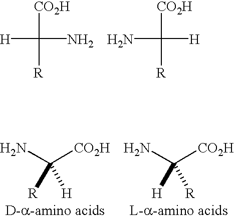

A “reversed” or “retro” peptide sequence as disclosed herein refers to that part of an overall sequence of covalently-bonded amino acid residues (or analogs or mimetics thereof) wherein the normal carboxyl-to amino direction of peptide bond formation in the amino acid backbone has been reversed such that, reading in the conventional left-to-right direction, the amino portion of the peptide bond precedes (rather than follows) the carbonyl portion. See, generally, Goodman, M. and Chorev, M. Accounts of Chem. Res. 1979, 12, 423.

The reversed orientation peptides described herein include (a) those wherein one or more amino-terminal residues are converted to a reversed (“rev”) orientation (thus yielding a second “carboxyl terminus” at the left-most portion of the molecule), and (b) those wherein one or more carboxyl-terminal residues are converted to a reversed (“rev”) orientation (yielding a second “amino terminus” at the right-most portion of the molecule). A peptide (amide) bond cannot be formed at the interface between a normal orientation residue and a reverse orientation residue.

Therefore, certain reversed peptide compounds of the invention can be formed by utilizing an appropriate amino acid mimetic moiety to link the two adjacent portions of the sequences depicted above utilizing a reversed peptide (reversed amide) bond. In case (a) above, a central residue of a di-keto compound may conveniently be utilized to link structures with two amide bonds to achieve a peptidomimetic structure. In case (b) above, a central residue of a diamino compound will likewise be useful to link structures with two amide bonds to form a peptidomimetic structure.

The reversed direction of bonding in such compounds will generally, in addition, require inversion of the enantiomeric configuration of the reversed amino acid residues in order to maintain a spatial orientation of side chains that is similar to that of the non-reversed peptide. The configuration of amino acids in the reversed portion of the peptides is preferably (D), and the configuration of the non-reversed portion is preferably (L). Opposite or mixed configurations are acceptable when appropriate to optimize a binding activity.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof (e.g. the ability to bind to the ECBP binding domain), wherein one or more simple variations of substituents are made which do not adversely affect the efficacy of the compound in binding to the ECBP binding domain. In general, the compounds of the present invention may be prepared by the methods illustrated in the general reaction schemes as, for example, described below, or by modifications thereof, using readily available starting materials, reagents and conventional synthesis procedures. Thus, the contemplated equivalents include peptidomimetic or non-peptide small molecule binders of the ECBP domain. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

As used herein, the term “pharmaceutically acceptable” refers to a carrier medium which does not interfere with the effectiveness of the biological activity of the active ingredients and which is not excessively toxic to the hosts of the concentrations of which it is administered. The administration(s) may take place by any suitable technique, including subcutaneous and parenteral administration, preferably parenteral. Examples of parenteral administration include intravenous, intraarterial, intramuscular, and intraperitoneal, with intravenous being preferred.

As used herein, the term “prophylactic or therapeutic” treatment refers to administration to the host of the medical condition. If it is administered prior to exposure to the condition, the treatment is prophylactic (i.e., it protects the host against infection), whereas if administered after infection or initiation of the disease, the treatment is therapeutic (i.e., it combats the existing infection or cancer).

The term “cell-proliferative disorder” denotes malignant as well as nonmalignant cell populations which morphologically often appear to differ from the surrounding tissue.

III. Exemplary Uses of ECBP Sequences

Merely to illustrate, potential applications of the ECBP therapeutics of the present invention include (by category):

A. Diagnostics:

Fluorescence-based cystoscopy for directed biopsy of otherwise invisible neointima or cancerous lesions

Staging of neointima or cancer by radioscintigraphy

Staging of neointima or cancer by MRI

Staging of neointima or cancer by optical imaging

B. Treatment:

Certain of the compounds of the invention possess anti-angiogenic activity, e.g., are capable of inhibiting angiogenesis by inhibiting proliferation of endothelial cells, and are thereby treating diseases and processes that are mediated by angiogenesis including, but not limited to, hemangioma, solid tumors, leukemia, metastasis, telangiectasia, psoriasis, scleroderma, pyogenic granuloma, myocardial angiogenesis, plaque neovascularization, coronary collaterals, cerebral collaterals, arteriovenous malformations, ischemic limb angiogenesis, corneal diseases, rubeosis, neovascular glaucoma, diabetic retinopathy, retrolental fibroplasia, arthritis, diabetic neovascularization, macular degeneration, wound healing, peptic ulcer, Helicobacter related diseases, fractures, keloids, vasculogenesis, hematopoiesis, ovulation, menstruation, placentation, and cat scratch fever.

To further illustrate, as angiogenesis inhibitors, such compounds are useful in the treatment of both primary and metastatic solid tumors and carcinomas of the breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries, choriocarcinoma and gestational trophoblastic disease; male genital tract including prostate, seminal vesicles, testes and germ cell tumors; endocrine glands including thyroid, adrenal, and pituitary; skin including hemangiomas, melanomas, sarcomas arising from bone or soft tissues and Kaposi's sarcoma; tumors of the brain, nerves, eyes, and meninges including astrocytomas, gliomas, glioblastomas, retinoblastomas, neuromas, neuroblastomas, Schwannomas and meningiomas; solid tumors arising from hematopoietic malignancies such as leukemias and including chloromas, plasmacytomas, plaques and tumors of mycosis fungoides and cutaneous T-cell lymphoma/leukemia; lymphomas including both Hodgkin's and non-Hodgkin's lymphomas; prophylaxis of autoimmune diseases including rheumatoid, immune and degenerative arthritis; ocular diseases including diabetic retinopathy, retinopathy of prematurity, corneal graft rejection, retrolental fibroplasia, neovascular glaucoma, rubeosis, retinal neovascularization due to macular degeneration and hypoxia; abnormal neovascularization conditions of the eye; skin diseases including psoriasis; blood vessel diseases including hemagiomas and capillary proliferation within atherosclerotic plaques; Osler-Webber Syndrome; myocardial angiogenesis; plaque neovascularization; telangiectasia; hemophiliac joints; angiofibroma; wound granulation; diseases characterized by excessive or abnormal stimulation of endothelial cells including intestinal adhesions, Crohn's disease, atherosclerosis, scleroderma and hypertrophic scars (i.e. keloids) and diseases which have angiogenesis as a pathologic consequence including cat scratch disease (Rochele minalia quintosa) and ulcers (Helicobacter pylori). Another use is as a birth control agent which inhibits ovulation and establishment of the placenta.

The anti-angiogenic compounds of the present invention may also be useful for the prevention of metastases from the tumors described above either when used alone or in combination with radiotherapy and/or other chemotherapeutic treatments conventionally administered to patients for treating angiogenic diseases. For example, when used in the treatment of solid tumors, compounds of the present invention may be administered with chemotherapeutic agents such as alpha inteferon, COMP (cyclophosphamide, vincristine, methotrexate and prednisone), etoposide, mBACOD (methortrexate, bleomycin, doxorubicin, cyclophosphamide, vincristine and dexamethasone), PRO-MACE/MOPP (prednisone, methotrexate (w/leucovin rescue), doxorubicin, cyclophosphamide, taxol, etoposide/mechlorethamine, vincristine, prednisone and procarbazine), vincristine, vinblastine, angioinhibins, TNP-470, pentosan polysulfate, platelet factor 4, angiostatin, LM-609, SU-101, CM-101, Techgalan, thalidomide, SP-PG and the like. Other chemotherapeutic agents include alkylating agents such as nitrogen mustards including mechloethamine, melphan, chlorambucil, cyclophosphamide and ifosfamide; nitrosoureas including carmustine, lomustine, semustine and streptozocin; alkyl sulfonates including busulfan; triazines including dacarbazine; ethyenimines including thiotepa and hexamethylmelamine; folic acid analogs including methotrexate; pyrimidine analogues including 5-fluorouracil, cytosine arabinoside; purine analogs including 6-mercaptopurine and 6-thioguanine; antitumor antibiotics including actinomycin D; the anthracyclines including doxorubicin, bleomycin, mitomycin C and methramycin; hormones and hormone antagonists including tamoxifen and cortiosteroids and miscellaneous agents including cisplatin and brequinar. For example, a tumor may be treated conventionally with surgery, radiation or chemotherapy and kringle 5 administration with subsequent kringle 5 adminsteration to extend the dormancy of micrometastases and to stabilize and inhibit the growth of any residual primary tumor.

In addition to these disorders, the antagonists may also be employed to treat retinopathy associated with diabetes, rheumatoid arthritis, osteoarthritis, macular degeneration, glaucoma, Keloid formation, ulcerative colitis, Krohn's disease, psoriasis, and other conditions caused are exacerbated by increased angiogenic activity. The antagonists may be employed in a composition with a pharmaceutically acceptable carrier, e.g., as described herein.

In certain preferred embodiments, the subject methods employ embodiment(s) of ECBP therapeutics have ED₅₀ for inhibiting endothelial cell proliferation and/or migration of 10⁻⁴M or less, and even more preferably 10⁻⁵M, 10⁻⁶M, 10⁻⁷M or even 10⁻⁸M or less.

In other embodiments, the certain of the subject ECBP therapeutics promote angiogenesis, e.g., are angiogenic, and be used to promote angiogenesis, such as, to repair damage of myocardial tissue as a result of myocardial infarction. Such methods may also include the repair of the cardiac vascular system after ischemia including the growth of collateral vasculature. Methods utilizing ECBP therapeutics may be employed to stimulate the growth of transplanted tissue and collateral vasculature where coronary bypass surgery is performed. Methods may also treat damaged vascular tissue as a result of coronary artery disease and peripheral or central nervous system vascular disease or ischemia.

Methods of the invention may also promote wound healing, particularly to re-vascularize damaged tissues or stimulate collateral blood flow during ischemia and where new capillary angiogenesis is desired. Other methods of the invention may be employed to treat full-thickness wounds such as dermal ulcers, including pressure sores, venous ulcers, and diabetic ulcers. In addition, methods employing ECBP therapeutics may be employed to treat full-thickness burns and injuries where a skin graft or flap is used to repair such burns and injuries. Such ECBP therapeutics may also be employed for use in plastic surgery, for example, for the repair of lacerations, burns, or other trauma. In urology, methods of the invention may assist in recovery of erectile function. In the field of female reproductive health, methods of the invention may assist in the modulation of menstruation, ovulation, endometrial lining formation and maintenance, and placentation.

Since angiogenesis is important in keeping wounds clean and non-infected, methods may be employed in association with surgery and following the repair of cuts. They may also be employed for the treatment of abdominal wounds where there is a high risk of infection. Methods using ECBP therapeutics described herein may be employed for the promotion of endothelialization in vascular graft surgery. In the case of vascular grafts using either transplanted or synthetic material, ECBP therapeutics can be applied to the surface of the graft or at the junction to promote the growth of vascular smooth muscle and adventitial cells in conjunction with endothelial cells.

Methods of the invention may also be employed to coat artificial prostheses or natural organs which are to be transplanted in the body to minimize rejection of the transplanted material and to stimulate vascularization of the transplanted materials and may also be employed for vascular tissue repair, for example, that occurring during arteriosclerosis and required following balloon angioplasty where vascular tissues are damaged. Specifically, methods of the invention may be employed to promote recovery from arterial wall injury and thereby inhibit restenosis.

These therapeutic agents may be administered by any route which is compatible with the particular agent employed. The ECBP therapeutic agents of the invention may be provided to an individual by any suitable means, preferably directly (e.g., locally, as by injection or topical administration to a tissue locus) or systemically (e.g., parenterally or orally). Where the agent is to be provided parenterally, such as by intravenous, intraarterial, subcutaneous, or intramuscular, administration, the agent preferably comprises part of an aqueous solution. The solution is physiologically acceptable so that in addition to delivery of the desired agent to the subject, the solution does not otherwise adversely affect the subject's electrolyte and/or volume balance. The aqueous medium for the ECBP therapeutic may comprise normal physiologic saline (e.g., 9.85% NaCl, 0.15 M, pH 7-7.4).

In certain preferred embodiments, the subject methods employ embodiment(s) of ECBP therapeutics have EC₅₀ for promoting endothelial cell proliferation and/or migration of 10⁻⁴ M or less, and even more preferably 10⁻⁵M, 10⁻⁶ M, 10⁻⁷ M or even 10⁻⁸ M or less.

For therapy associated with modulating angiogenesis, one may administer the present ECBP therapeutics (or derivatives) in conjunction with one or more pharmaceutical compositions used for treating other clinical complications of the need for angiogenic modulation, such as those used for treatment of cancer (e.g., chemotherapeutics), cachexia, high blood pressure, high cholesterol, and other adverse conditions. Administration may be simultaneous or may be in seriatim. Similarly, one may administer more than one ECBP therapeutic (or derivatives), having the same or differing mode of action, to attain an additive or synergistic effect on angiogenesis.

C. Prevention:

Identification of early lesions for preventative removal

Adjuvant treatment after definitive resection

D. Discovery

Biological identification of the compounds that mitigate the inhibitory effects of the peptide—i.e. to screen for growth factors or other compounds capable of overcoming or reversing the inhibition of endothelial cell proliferation.

The subject peptides can also be used to identify compounds, such as small organic molecules, which can mimic the effect of the peptide on endothelial cell proliferation and/or migration.

IV. Description of Certain Preferred Embodiments

A. Generating Variants of ECBP Sequences

As mentioned above, the inventive peptide compositions, including other peptidomimetics, non-peptide small molecules, genes and recombinant polypeptides may be generated using combinatorial techniques available in the art for generating combinatorial libraries of small organic/peptide libraries. See, for example, Blondelle et al. (1995)Trends Anal. Chem. 14:83; the Affymax U.S. Pat. Nos. 5,359,115 and 5,362,899; the Ellman U.S. Pat. No. 5,288,514; the Still et al. PCT publication WO 94/08051; Chen et al. (1994) JACS 116:2661; Kerr et al. (1993) JACS 115:252; PCT publications WO92/10092, WO93/09668 and WO91/07087; and the Lerner et al. PCT publication WO93/20242).

In a preferred embodiment, the combinatorial peptide library is produced by way of a degenerate library of genes encoding a library of polypeptides which each include at least a portion of potential ECBP sequences. For instance, a mixture of synthetic oligonucleotides can be enzymatically ligated into gene sequences such that the degenerate set of potential ECBP binding nucleotide sequences are expressible as individual polypeptides, or alternatively, as a set of larger fusion proteins (e.g., for phage display) containing the set of ECBP sequences therein.

There are many ways by which the gene library of potential ECBP binding homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential ECBP sequences. The synthesis of degenerate oligonucleotides is well known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp. 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

A wide range of techniques are known in the art for screening gene products of combinatorial libraries made by point mutations. Such techniques will be generally adaptable for rapid screening of the gene libraries generated by the combinatorial mutagenesis of ECBP sequences. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Such illustrative assays are amenable to high throughput analysis as necessary to screen large numbers of degenerate sequences created by combinatorial mutagenesis techniques.

In an illustrative embodiment of a screening assay, the ECBP binding gene library can be expressed as a fusion protein on the surface of a viral particle. For instance, in the filamentous phage system, foreign peptide sequences can be expressed on the surface of infectious phage, thereby conferring two significant benefits. First, since these phage can be applied to endothelial cancer cells at very high concentrations, a large number of phage can be screened at one time. Second, since each infectious phage displays the combinatorial gene product on its surface, if a particular phage is recovered from the cancer cells in low yield, the phage can be amplified by another round of infection. The group of almost identical E. coli filamentous phages M13, fd, and f1 are most often used in phage display libraries, as either of the phage gIII or gVIII coat proteins can be used to generate fusion proteins without disrupting the ultimate packaging of the viral particle (Ladner et al. PCT publication WO 90/02909; Garrard et al., PCT publication WO 92/09690; Marks et al. (1992) J. Biol. Chem. 267:16007-16010; Griffths et al. (1993) EMBO J 12:725-734; Clackson et al. (1991) Nature 352:624-628; and Barbas et al. (1992) PNAS 89:4457-4461).

For example, the recombinant phage antibody system (RPAS, Pharmacia Catalog number 27-9400-01) can be easily modified for use in expressing and screening ECBP binding motif combinatorial libraries of the present invention. For instance, the pCANTAB 5 phagemid of the RPAS kit contains the gene which encodes the phage gIII coat protein. The ECBP binding combinatorial gene library can be cloned into the phagemid adjacent to the gill signal sequence such that it will be expressed as a gIII fusion protein. After ligation, the phagemid is used to transform competent E. coli TG1 cells. Transformed cells are subsequently infected with M13KO7 helper phage to rescue the phagemid and its candidate ECBP binding gene insert. The resulting recombinant phage contain phagemid DNA encoding a specific candidate ECBP, and display one or more copies of the corresponding fusion coat protein. The phage-displayed candidate proteins which are capable of, for example, binding to endothelial cells, are selected or enriched by panning. For instance, the phage library can be panned on endothelial cells, and unbound phage washed away from the cells. The bound phage is then isolated, and if the recombinant phage express at least one copy of the wild type gIII coat protein, they will retain their ability to infect E. Coli. Thus, successive rounds of reinfection of E. coli, and panning will greatly enrich for ECBP binding homologs.

B. ECBP Peptidomimetics

In other embodiments, the subject ECBP binding therapeutics are peptidomimetics of the ECBP. Peptidomimetics are compounds based on, or derived from, peptides and proteins. The ECBP binding peptidomimetics of the present invention typically can be obtained by structural modification of a known ECBP sequence using unnatural amino acids, conformational restraints, isosteric replacement, and the like. The subject peptidomimetics constitute the continuum of structural space between peptides and non-peptide synthetic structures; ECBP binding peptidomimetics may be useful, therefore, in delineating pharmacophores and in helping to translate peptides into nonpeptide compounds with the activity of the parent ECBPs.

Moreover, as is apparent from the present disclosure, mimetopes of the subject ECBPs can be provided. Such peptidomimetics can have such attributes as being non-hydrolyzable (e.g., increased stability against proteases or other physiological conditions which degrade the corresponding peptide), increased specificity and/or potency, and increased cell permeability for intracellular localization of the peptidomimetic. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988, p123), C-7 mimics (Huffman et al. in Peptides: Chemistry and Biologyy, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), β-aminoalcohols (Gordon et al. (1985) Biochem Biophys Res Commun126:419; and Dann et al. (1986) Biochem Biophys Res Commun 134:71), diaminoketones (Natarajan et al. (1984) Biochem Biophys Res Commun 10 124:141), and methyleneamino-modified (Roark et al. in Peptides: Chemistry and Biology, G. R. Marshall ed., ESCOM Publisher: Leiden, Netherlands, 1988, p134). Also, see generally, Session III: Analytic and synthetic methods, in Peptides: Chemistry and Biology, G. R. Marshall ed., ESCOM Publisher: Leiden, Netherlands, 1988)

In addition to a variety of sidechain replacements which can be carried out to generate the subject ECBP binding peptidomimetics, the present invention specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides.

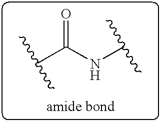

Examples of Surrogates

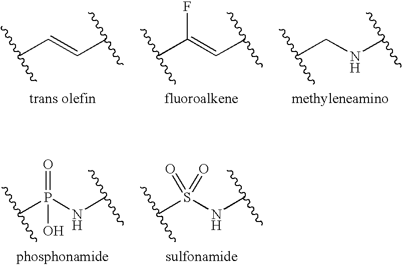

Additionally, peptidomimietics based on more substantial modifications of the backbone of the E2 peptide can be used. Peptidomimetics which fall in this category include (i) retro-inverso analogs, and (ii) N-alkyl glycine analogs (so-called peptoids).

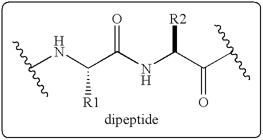

Examples of Analogs

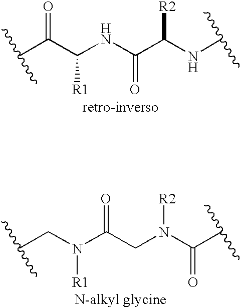

Furthermore, the methods of combinatorial chemistry are being brought to bear, e.g., by G. L. Verdine at Harvard University, on the development of new peptidomimetics. For example, one embodiment of a so-called “peptide morphing” strategy focuses on the random generation of a library of peptide analogs that comprise a wide range of peptide bond substitutes.

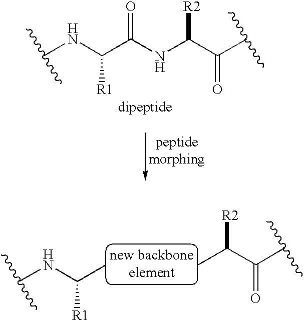

In an exemplary embodiment, the peptidomimetic can be derived as a retro-inverso analog of the peptide

Retro-inverso analogs can be made according to the methods known in the art, such as that described by the Sisto et al. U.S. Pat. No. 4,522,752. As a general guide, sites which are most susceptible to proteolysis are typically altered, with less susceptible amide linkages being optional for mimetic switching The final product, or intermediates thereof, can be purified by HPLC.

In another illustrative embodiment, the peptidomimetic can be derived as a retro-enatio analog of a particular ECBP sequence. Retro-enantio analogs such as this can be synthesized commercially available D-amino acids (or analogs thereof) and standard solid- or solution-phase peptide-synthesis techniques.

In still another illustrative embodiment, trans-olefin derivatives can be made for any of the subject polypeptides. A trans-olefin analog of ECBP can be synthesized according to the method of Y. K. Shue et al. (1987) Tetrahedron Letters 28:3225 and also according to other methods known in the art. It will be appreciated that variations in the cited procedure, or other procedures available, may be necessary according to the nature of the reagent used.

It is further possible couple the pseudodipeptides synthesized by the above method to other pseudodipeptides, to make peptide analogs with several olefinic functionalities in place of amide functionalities. For example, pseudodipeptides corresponding to certain di-peptide sequences could be made and then coupled together by standard techniques to yield an analog of the ECBP which has alternating olefinic bonds between residues.

Still another class of peptidomimetic derivatives include phosphonate derivatives. The synthesis of such phosphonate derivatives can be adapted from known synthesis schemes. See, for example, Loots et al. in Peptides: Chemistry and Biology, (Escom Science Publishers, Leiden, 1988, p. 118); Petrillo et al. in Peptides: Structure and Function (Proceedings of the 9th American Peptide Symposium, Pierce Chemical Co. Rockland, Ill., 1985).

Many other peptidomimetic structures are known in the art and can be readily adapted for use in the subject ECBP binding peptidomimetics. To illustrate, the ECBP binding peptidomimetic may incorporate the 1-azabicyclo[4.3.0]nonane surrogate ( see Kim et al, (1997) J. Org. Chem. 62:2847), or an N-acyl piperazic acid (see Xi et al. (1998) J. Am. Chem. Soc. 120:80), or a 2-substituted piperazine moiety as a constrained amino acid analogue (see Williams et al. (1996) J. Med. Chem. 39:1345-1348). In still other embodiments, certain amino acid residues can be replaced with aryl and bi-aryl moieties, e.g., monocyclic or bicyclic aromatic or heteroaromatic nucleus, or a biaromatic, aromatic-heteroaromatic, or biheteroaromatic nucleus.

The subject ECBP binding peptidomimetics can be optimized by, e.g., combinatorial synthesis techniques combined with such high throughput screening as described above using affinity maturation of the library on endothelial cells.

Moreover, other examples of mimetopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of natural and synthetic compounds for compounds capable of binding to the ECBP binding domain or inhibiting the interaction between the ECBP binding domain and the natural ligand. A mimetope can also be obtained, for example, from libraries of natural and synthetic compounds, in particular, chemical or combinatorial libraries (i.e., libraries of compounds that differ in sequence or size but that have the same building blocks). A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimetopes by, for example, computer modeling. the predicted mimetope structures can then be produced by, for example, chemical synthesis, recombinant DNA technology, or by isolating a mimetope from a natural source (e.g., plants, animals, bacteria and fungi).

C. Chimeric ECBP Binding Peptides and Peptidomimetics

In one aspect, the invention provides chimeric proteins which include one or more ECBPs fused to one or more additional protein domains. In one embodiment, the chimeric protein includes one ECBP. In other embodiments, the chimeric activator comprises two or more ECBPs, three or more, five or more, or ten or more ECBPs that are covalently linked. When referring to a polypeptide comprising an ECBP it is meant that the polypeptide comprises the amino acid sequence of an ECBP covalently linked to other amino acids or peptides to form one polypeptide. The order of the ECBP(s) relative to each other and relative to the other domains of the fusion protein can be as desired.

Techniques for making the subject fusion proteins are adapted from well-known procedures. Essentially, the joining of various DNA fragments coding for different polypeptide sequences is performed in accordance with conventional techniques, employing blunt-ended or stagger-ended termini for ligation, restriction enzyme digestion to provide for appropriate termini, filling in of cohesive ends as appropriate, alkaline phosphatase treatment to avoid undesirable joining, and enzymatic ligation. Alternatively, the fusion gene can be synthesized by conventional techniques including automated DNA synthesizers. In another method, PCR amplification of gene fragments can be carried out using anchor primers which give rise to complementary overhangs between two consecutive gene fragments. Amplification products can subsequently be annealed to generate a chimeric gene sequence (see, for example, Current Protocols in Molecular Biology, Eds. Ausubel et al. John Wiley & Sons: 1992).

In certain preferred embodiments, the subject peptide sequences are included as part of a fusion protein with a serum protein, being added at either the N- or C-terminus of the proteins, or at one or more internal sites. Examples of serum proteins which can be used in the present invention include albumin, α-globulins, β-globulins, γ-globulins, haptoglobin, transthyretin, collagen, α2 macroglobulin, β2 microglobulin, C Reactive Protein, apolipoproteins, lipoproteins, cathepsins amylase, antichymotrypsin, ferritin, α fetoprotein, elastin and fibronectin and coagulation factors including fibrinogen, fibrin, thrombin, ceruloplasmin, antiplasmin and antithrombin III, and the like.

In other embodiments, the subject peptide sequences are provided in the form of cyclic peptides. The term “cyclic peptide” as used herein refers to cyclic derivatives of peptides containing one or more intramolecular binds to form a macrocyclic structure. The intramolecular bond may be a backbone-to-backbone, sidechain-to-backbone or sidechain-to-sidechain bond (i.e., terminal functional groups of a linear peptide and/or sidechain functional groups of a terminal or interior residue may be linked to achieve cyclization). Preferred intramolecular bonds include, but are not limited to: disulfide bonds; amide bonds between terminal functional groups, between residue side chains or between one terminal functional groups and one residue side chain; thioether bonds; and Δ1, Δ1′-ditryptophan. Merely to illustrate, the subject cyclic peptides can be generated by incorporation of an intramolecular disulfide bond, i.e., —S—S—, an intramolecular amide bond, e.g., —CONH— or —NHCO—, or intramolecular S-alkyl bonds.

Strategies for the preparation of circular polypeptides from linear precursors are well known. Approaches include chemical (Camarero, et al., (1998) Angew. Chem. Int. Ed., 37:347-349; Tamand Lu (1998) Prot. Sci. 77:1583-1592; Camarero and Muir (1997) Chem. Commun., 1997:1369-1370; and Zhang and Tam (1997) J. Am. Chem. Soc. 119:2363-2370) and enzymatic (Jackson et al., (1995) J. Am. Chem. Soc., 117:819-820) intramolecular ligation methods which allow linear synthetic peptides to be efficiently cyclized under aqueous conditions.

To further illustrate, the cyclic derivatives containing an intramolecular disulfide bond may be prepared by conventional solid phase synthesis while incorporating suitable S-protected cysteine or homocysteine residues at the positions selected for cyclization such as the amino and carboxy terminals of the peptides, with the option of including spacing residues, such as alanine, glycine or non-natural amino acids such as 6-aminocaproic acid, between the terminal residue and the linking residue. The linking residues may then be linked together using known techniques to form cyclicized peptide derivatives. For example, a cyclic peptide can be prepared by elongation from a linear peptide by selective removal of the S-protecting groups with a consequent on-support oxidation of free corresponding two SH-functions, to form S—S bonds, followed by conventional removal of the product from the support and appropriate purification procedure, or by removal of the peptide from the support along with complete side-chain deprotection, followed by oxidation of the free SH-functions in highly dilute aqueous solution.

In another illustrative embodiment, air oxidation of thiols can generate disulfide linkages over a period of several days using either basic or neutral aqueous media. The peptide is used in high dilution to minimize aggregation and intermolecular side reactions. This method suffers from the disadvantage of being slow but has the advantage of only producing H₂O as a side product. Alternatively, strong oxidizing agents such as I₂ and K₃Fe(CN)₆ can be used to form disulfide linkages. Those of ordinary skill in the art will recognize that care must be taken not to oxidize the sensitive side chains of Met, Tyr, Trp or His. Cyclic peptides produced by this method can be purified using standard techniques, but this oxidation is applicable at acid pHs. Oxidizing agents also allow concurrent deprotection/oxidation of suitable S-protected linear precursors to avoid premature, nonspecific oxidation of free cysteine.

DMSO, unlike I₂ and K₃Fe(CN)₆, is a mild oxidizing agent which does not cause oxidative side reactions of the nucleophilic amino acids mentioned above. DMSO is miscible with H₂O at all concentrations, and oxidations can be performed at acidic to neutral pHs with harmless byproducts. Methyltrichlorosilane-diphenylsulfoxide may alternatively be used as an oxidizing agent, for concurrent deprotection/oxidation of S-Acm, S-Tacm or S-t-Bu of cysteine without affecting other nucleophilic amino acids. There are no polymeric products resulting from intermolecular disulfide bond formation. Suitable thiol-containing residues for use in such oxidation methods include, but are not limited to, cysteine, β,β-dimethyl cysteine (penicillamine or Pen), β,β-tetramethylene cysteine (Tmc), β,β-pentamethylene cysteine (Pmc), β-mercaptopropionic aid (Mpr), β,β-pentamethylene-β-mercaptopropionic acid (Pmp), 2-mercaptobenzene, 2-mercaptoaniline and 2-mercaptoproline.

Head-to-tail (backbone) peptide cyclization has been used to rigidify structure and improve in vivo stability of small bioactive peptides (see Camarero and Muir, (1999) J. Am. Chem. Soc., 121:5597-5598). The cyclic derivatives containing an intramolecular amide bond may be prepared by conventional solid phase synthesis while incorporating suitable amino and carboxyl side-chain protected amino acid derivatives at the positions selected for cyclization.

For instance, cyclization may be achieved by amide bond formation. To further illustrate, a peptide bond may be formed between terminal functional groups (i.e., the amino and carboxy termini of a linear peptide prior to cyclization), with or without an N-terminal acetyl group and/or a C-terminal amide. Within another such embodiment, the linear peptide comprises a D-amino acid. Alternatively, cyclization may be accomplished by linking one terminus and a residue side chain or using two side chains, with or without an N-terminal acetyl group and/or a C-terminal amide. Residues capable of forming a lactam bond include lysine, ornithine, α-amino adipic acid, m-aminomethylbenzoic acid, α,β-diaminopropionic acid, glutamate or aspartate.

Other methods for forming amide bonds are well known in the art and are based on well established principles of chemical reactivity. Within one such method, carbodiimide-mediated lactam formation can be accomplished by reaction of the carboxylic acid with DCC, DIC, EDAC or DCCI, resulting in the formation of an O-acylurea that can be reacted immediately with the free amino group to complete the cyclization. The formation of the inactive N-acylurea, resulting from O=>N migration, can be circumvented by converting the O-acylurea to an active ester by reaction with an N-hydroxy compound such as 1-hydroxybenzotriazole, 1-hydroxysuccinimide, 1-hydroxynorbornene carboxamide or ethyl 2-hydroximino-2-cyanoacetate. In addition to minimizing O=>N migration, these additives also serve as catalysts during cyclization and assist in lowering racemization. Alternatively, cyclization can be performed using the azide method, in which a reactive azide intermediate is generated from an alkyl ester via a hydrazide. Hydrazinolysis of the terminal ester necessitates the use of a t-butyl group for the protection of side chain carboxyl functions in the acylating component. This limitation can be overcome by using diphenylphosphoryl acid (DPPA), which furnishes an azide directly upon reaction with a carboxyl group. The slow reactivity of azides and the formation of isocyanates by their disproportionation restrict the usefulness of this method. The mixed anhydride method of lactam formation is widely used because of the facile removal of reaction by-products. The anhydride is formed upon reaction of the carboxylate anion with an alkyl chloroformate or pivaloyl chloride. The attack of the amino component is then guided to the carbonyl carbon of the acylating component by the electron donating effect of the alkoxy group or by the steric bulk of the pivaloyl chloride t-butyl group, which obstructs attack on the wrong carbonyl group. Mixed anhydrides with phosphoric acid derivatives have also been successfully used. Alternatively, cyclization can be accomplished using activated esters. The presence of electron withdrawing substituents on the alkoxy carbon of esters increases their susceptibility to aminolysis. The high reactivity of esters of p-nitrophenol, N-hydroxy compounds and polyhalogenated phenols has made these “active esters” useful in the synthesis of amide bonds. The last few years have witnessed the development of benzotriazolyloxytris-(dimethylamino)phosphonium hexafluorophosphonate (BOP) and its congeners as advantageous coupling reagents. Their performance is generally superior to that of the well established carbodiimide amide bond formation reactions.

The cyclic derivatives containing intramolecular —S-alkyl bonds may be prepared by conventional solid phase synthesis while incorporating an amino acid residue with a suitable amino-protected side chain, and a suitable S-protected cysteine or homocysteine residue at the positions selected for cyclization.

Within a further embodiment, a thioether linkage may be formed between the side chain of a thiol-containing residue and an appropriately derivatized α-amino acid. By way of example, a lysine side chain can be coupled to bromoacetic acid through the carbodiimide coupling method (DCC, EDAC) and then reacted with the side chain of any of the thiol containing residues mentioned above to form a thioether linkage. In order to form dithioethers, any two thiol containing side-chains can be reacted with dibromoethane and diisopropylamine in DMF. Cyclization may also be achieved using Δ1, Δ1′-ditryptophan

Another solution to generating the cyclic peptides of the present invention is to generate circular recombinant peptides using a native chemical ligation approach. For instance, inteins (internal proteins) can be used to catalyze head-to-tail peptide ligation in vivo (see, for example, Evans, et al. (1999) J. Biol. Chem. 5.274:18359-18363; Iwai et al. (1999) FEBS Lett. 459:166-172; Wood, et al. (1999) Nature Biotechnology 17:889-892; Camarero et al (1 999) J. Am. Chem. Soc. 121:5597-5598; and Scott, et al. (1999) Proc. Natl. Acad. Sci. USA 96:13638-13643). Inteins are self-splicing proteins that occur as in-frame insertions in specific host proteins. In a self-splicing reaction, inteins excise themselves from a precursor protein, while the flanking regions, the exteins, become joined to restore host gene function. Inteins can also catalyze a trans-ligation self-splicing reaction. Approaches making use of the trans-ligation reaction include splitting the intein into two parts and reassembling the two parts in vitro, each fused to a different extein (Southworth, et al., (1998) EMBO J. 17:918-926). A somewhat different approach uses an intein domain, and the reaction is then triggered with a thiolate nucleophile, such as DTT (Xu, et al., (1998) Protein Sci., 7:2256-2264). Fusing the split intein sequences to the carboxy and amino termini of a peptide, e.g., by generating a chimeric coding sequence, permits such split intein-mediated circular ligation reactions to be used to recombinantly generate the head-to-tail cyclic peptides of the present invention.

In certain embodiments, polyanionic or polycatonic binding agents such as oligonucleotides, heparin, lentinan and similar polysaccharide chains, polyamino peptides such as polyaspartate, polyglutamate, polylysine and polyarginine, or other binding agents which maintain a number of either negative or positive charges over their structure at physiological pH's, can be used to specifically bind the subject ECBPs or peptidomimetics. In certain preferred embodiments, a polyanionic component is used, such as heparin, pentosan polysulfate, polyaspartate, polyglutamate, chondroitin sulfate, heparan sulfate, citrate, nephrocalcin, or osteopontin, to name but a few.

(i) Additional Domains and Linkers

Additional domains may be included in the subject fusion proteins of this invention. For example, the fusion proteins may include domains that facilitate their purification, e.g. “histidine tags” or a glutathione-S-transferase domain. They may include “epitope tags” encoding peptides recognized by known monoclonal antibodies for the detection of proteins within cells or the capture of proteins by antibodies in vitro.

It may be necessary in some instances to introduce an unstructured polypeptide linker region between an ECBP and other portions of the chimeric protein. The linker can facilitate enhanced flexibility of the fusion protein. The linker can also reduce steric hindrance between any two fragments of the fusion protein. The linker can also facilitate the appropriate folding of each fragment to occur. The linker can be of natural origin, such as a sequence determined to exist in random coil between two domains of a protein. An exemplary linker sequence is the linker found between the C-terminal and N-terminal domains of the RNA polymerase a subunit. Other examples of naturally occurring linkers include linkers found in the lcI and LexA proteins. Alternatively, the linker can be of synthetic origin. For instance, the sequence (Gly₄Ser)₃ can be used as a synthetic unstructured linker. Linkers of this type are described in Huston et al. (1988) PNAS 85:4879; and U.S. Pat. No. 5,091,513.

In some embodiments it is preferable that the design of a linker involve an arrangement of domains which requires the linker to span a relatively short distance, preferably less than about 10 Angstroms. However, in certain embodiments, depending, e.g., upon the selected domains and the configuration, the linker may span a distance of up to about 50 Angstrom.

Within the linker, the amino acid sequence may be varied based on the preferred characteristics of the linker as determined empirically or as revealed by modeling. For instance, in addition to a desired length, modeling studies may show that side groups of certain amino acids may interfere with the biological activity of the fusion protein. Considerations in choosing a linker include flexibility of the linker, charge of the linker, and presence of some amino acids of the linker in the naturally-occurring subunits. The linker can also be designed such that residues in the linker contact DNA, thereby influencing binding affinity or specificity, or to interact with other proteins. For example, a linker may contain an amino acid sequence which can be recognized by a protease so that the activity of the chimeric protein could be regulated by cleavage. In some cases, particularly when it is necessary to span a longer distance between subunits or when the domains must be held in a particular configuration, the linker may optionally contain an additional folded domain.

(ii) Altering Viral Targeting

In certain embodiments, the present invention relates to the use of ECBPs and peptidomimetics to alter the infectivity spectrum of a viral particle, e.g., to enhance the specificity of a given viral particle for endothelial cells. Viral coat proteins can be modified with an ECBP or peptidomimetic or fusion protein by chemical conjugation. In other embodiments, the peptide sequence can be incorporated into a viral coat protein to create a fusion protein which displays the ECBP on the surface of an assembled viral particle.

For instance, the strategies for the modifying the infection spectrum of retroviral particles include coupling the subject ECBPs and peptidomimetics to the viral env protein (Roux et al., (1989) PNAS USA 86:9079-9083; Julan et al., (1992) J. Gen Virol 73:3251-3255; and Goud et al., (1983) Virology 163:251-254). Coupling can be in the form of the chemical cross-linking with a protein or other variety (e.g. lactose to convert the env protein to an asialoglycoprotein), as well as by generating fusion proteins.

Likewise, the ECBP sequence can be added to the coat protein of an adenoviral or adeno-associated viral system. In one preferred embodiment, the ECBP sequence is provided as part of a fusion protein, being included as part of the fiber knob protein. An adenovirus uses two separate cellular receptors, both of which must be present, to attach to and infect a cell (Wickham et al., (1993) Cell 73:309-319). First, the adenovirus fiber protein attaches the virus to a cell by binding to an as yet unidentified receptor. Then, the penton base binds to αv integrins, which are a family of heterodimeric cell-surface receptors that mediate cellular adhesion to the extracellular matrix molecules, as well as other molecules. Once an adenovirus is attached to a cell, it undergoes receptor-mediated internalization into clathrin-coated endocytic vesicles and is stepwise stripped down to the viral double-stranded genome, and then the genome (and some accompanying viral components) subsequently is transported to the cell nucleus, thus initiating infection. The fiber monomer consists of an amino terminal tail (which attaches noncovalently to the penton base), a shaft (whose length varies among different virus serotypes), and a carboxy terminal globular knob domain (which is necessary and sufficient for host cell binding) (Devaux et al., (1990) J. Molec. Biol., 215 :567-588; Green et al. (1983) EMBO J., 2:1357-1365; Henry et al. (1994) J. Virology 68 :5239-5246). The regions necessary for trimerization of fiber (which is required for penton base binding) also are located in the knob region of the protein (Henry et al. (1994), supra; Novelli et al., Virology, 185, 365-376 (1991)). The fiber, together with the hexon, determine the serotype specificity of the virus, and also comprise the main antigenic determinants of the virus (Watson et al., J. Gen. Virol., 69, 525-535 (1988)).

In certain preferred embodiments, one or more ECBP sequences are incorporated into the coding sequence for the fiber knob protein, such as corresponding to the HI loop, to produce a chimeric protein. The size of the insert and the final location are such as to not inhibit fiber trimerization nor ultimately disturb formation of the cell-binding site localized in the knob.

(iii) Toxins and Imaging Agents

In certain embodiments, the subject ECBPs and peptidomimetics can be covalently or non-covalently coupled to a cytotoxin or other cell proliferation inhibiting compound, in order to localize delivery of that agent to an endothelial cell. For instance, the agent can be selected from the group consisting of alkylating agents, enzyme inhibitors, proliferation inhibitors, lytic agents, DNA or RNA synthesis inhibitors, membrane permeability modifiers, DNA intercalators, metabolites, dichloroethylsulfide derivatives, protein production inhibitors, ribosome inhibitors, inducers of apoptosis, and neurotoxins.

Chemotherapeutics useful as active moieties which when conjugated to a modified ECBP or peptidomimetic or fusion proteins are specifically delivered to endothelial cells are typically, small chemical entities produced by chemical synthesis. Chemotherapeutics include cytotoxic and cytostatic drugs. Chemotherapeutics may include those which have other effects on cells such as reversal of the transformed state to a differentiated state or those which inhibit cell replication. Examples of known cytotoxic agents useful in the present invention are listed, for example, in Goodman et al., “The Pharmacological Basis of Therapeutics,” Sixth Edition, A. G. Gilman et al, eds./Macmillan Publishing Co. New York, 1980. These include taxanes, such as paclitaxel (Taxol®) and docetaxel (Taxotere®); nitrogen mustards, such as mechlorethamine, cyclophosphamide, melphalan, uracil mustard and chlorambucil; ethylenimine derivatives, such as thiotepa; alkyl sulfonates, such as busulfan; nitrosoureas, such as carmustine, lomustine, semustine and streptozocin; triazenes, such as dacarbazine; folic acid analogs, such as methotrexate; pyrimidine analogs, such as fluorouracil, cytarabine and azaribine; purine analogs, such as mercaptopurine and thioguanine; vinca alkaloids, such as vinblastine and vincristine; antibiotics, such as dactinomycin, daunorubicin, doxorubicin, bleomycin, mithramycin and mitomycin; enzymes, such as L-asparaginase; platinum coordination complexes, such as cisplatin; substituted urea, such as hydroxyurea; methyl hydrazine derivatives, such as procarbazine; adrenocortical suppressants, such as mitotane; hormones and antagonists, such as adrenocortisteroids (prednisone), progestins (hydroxyprogesterone caproate, medroprogesterone acetate and megestrol acetate), estrogens (diethylstilbestrol and ethinyl estradiol), antiestrogens (tamoxifen), and androgens (testosterone propionate and fluoxymesterone).

Drugs that interfere with intracellular protein synthesis can also be used; such drugs are known to these skilled in the art and include puromycin, cycloheximide, and ribonuclease.

Prodrugs forms of the chemotherapeutic moiety are especially useful in the present invention to generate an inactive precursor.

Most of the chemotherapeutic agents currently in use in treating cancer possess functional groups that are amenable to chemical cross-linking directly with an amine or carboxyl group of an ECBP. For example, free amino groups are available on methotrexate, doxorubicin, daunorubicin, cytosinarabinoside, bleomycin, gemcitabine, fludarabine, and cladribine while free carboxylic acid groups are available on methotrexate, melphalan, and chlorambucil. These functional groups, that is free amino and carboxylic acids, are targets for a variety of homobifunctional and heterobifunctional chemical cross-linking agents which can crosslink these drugs directly to a free amino group of an ECBP.

Peptide and polypeptide toxins are also useful as active moieties, and the present invention specifically contemplates embodiments wherein the ECBP moiety is coupled to a toxin. In certain preferred embodiments, the ECBP and toxin are both polypeptides and are provided in the form of a fusion protein. Toxins are generally complex toxic products of various organisms including bacteria, plants, etc. Examples of toxins include but are not limited to: ricin, ricin A chain (ricin toxin), Pseudomonas exotoxin (PE), diphtheria toxin (DT), Clostridium perfringens phospholipase C (PLC), bovine pancreatic ribonuclease (BPR), pokeweed antiviral protein (PAP), abrin, abrin A chain (abrin toxin), cobra venom factor (CVF), gelonin (GEL), saporin (SAP), modeccin, viscumin and volkensin.

The invention further contemplates embodiments the ECBP is coupled to a polymer or a functionalized polymer (e.g., a polymer conjugated to another molecule). Preferred examples include water soluble polymers, such as, polyglutamic acid or polyaspartic acid, conjugated to a drug such as a chemotherapeutic or antiangiogenic agent, including, for example, paclitaxel or docetaxel.

In certain preferred embodiments, particularly where the cytotoxic moiety is chemically cross-linked to the peptide moiety, the linkage is hydrolyzable from the peptide, e.g., such as may be provided by use of an amide or ester group in the linking moiety.

In certain embodiments, the subject peptides and peptidomimetics can be coupled with an agent useful in imaging endothelial cells. Such agents include: metals; metal chelators; lanthanides; lanthanide chelators; radiometals; radiometal chelators; positron-emitting nuclei; microbubbles (for ultrasound); liposomes; molecules microencapsulated in liposomes or nanosphere; monocrystalline iron oxide nanocompounds; magnetic resonance imaging contrast agents; light absorbing, reflecting and/or scattering agents; colloidal particles; fluorophores, such as near-infrared fluorophores. In many embodiments, such secondary functionality will be relatively large, e.g., at least 25 amu in size, and in many instances can be at least 50, 100 or 250 amu in size.

In certain preferred embodiments, the secondary functionality is a chelate moiety for chelating a metal, e.g., a chelator for a radiometal or paramagnetic ion. In preferred embodiments, it is a chelator for a radionuclide useful for radiotherapy or imaging procedures. Radionuclides useful within the present invention include gamma-emitters, positron-emitters, Auger electron-emitters, X-ray emitters and fluorescence-emitters, with beta- or alpha-emitters preferred for therapeutic use. Examples of radionuclides useful as toxins in radiation therapy include: ³²P, ³³P, ⁴³K, ⁴⁷Sc, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/^{81M}Kr, ^{87M}Sr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, 191Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi. Preferred therapeutic radionuclides include ¹⁸⁸Re, ¹⁸⁶Re, ²⁰³Pb, ²¹²Pb, ²¹²Bi, ¹⁰⁹Pd, ⁶⁴Cu, ⁶⁷Cu, ⁹⁰Y, ¹²⁵I, ¹³¹I, ⁷⁷Br, ²¹¹At, ⁹⁷Ru, ¹⁰⁵Rh, ¹⁹⁸Au and ¹⁹⁹Ag, ¹⁶⁶Ho or ¹⁷⁷Lu. Conditions under which a chelator will coordinate a metal are described, for example, by Gansow et al., U.S. Pat. Nos. 4,831,175, 4,454,106 and 4,472,509

^{99m}Tc is a particularly attractive radioisotope for therapeutic and diagnostic applications, as it is readily available to all nuclear medicine departments, is inexpensive, gives minimal patient radiation doses, and has ideal nuclear imaging properties. It has a half-life of six hours which means that rapid targeting of a technetium-labeled antibody is desirable. Accordingly, in certain preferred embodiments, the modified ECBPs includes a chelating agent for technium.

In still other embodiments, the secondary functionality can be a radiosensitizing agent, e.g., a moiety that increase the sensitivity of cells to radiation. Examples of radiosensitizing agents include nitroimidazoles, metronidazole and misonidazole (see: DeVita, V. T. Jr. in Harrison's Principles of Internal Medicine, p.68, McGraw-Hill Book Co., N.Y. 1983, which is incorporated herein by reference). The modified ECBP that comprises a radiosensitizing agent as the active moiety is administered and localizes at the metastasized cell. Upon exposure of the individual to radiation, the radiosensitizing agent is “excited” and causes the death of the cell.

There are a wide range of moieties which can serve as chelators and which can be derivatized to the ECBPs. For instance, the chelator can be a derivative of 1,4,7,10-tetraazacyclododecanetetraacetic acid (DOTA), ethylenediaminetetraacetic acid (EDTA), diethylenetriaminepentaacetic acid (DTPA) and 1-p-Isothiocyanato-benzyl-methyl-diethylenetriaminepentaacetic acid (ITC-MX). These chelators typically have groups on the side chain by which the chelator can be used for attachment to an ECBP. Such groups include, e.g., benzylisothiocyanate, by which the DOTA, DTPA or EDTA can be coupled to, e.g., an amine group of the ECBP.

In one embodiment, the chelate moiety is an “NₓS_{y}” chelate moiety. As defined herein, the term “NₓS_{y} chelates” includes bifunctional chelators that are capable of coordinately binding a metal or radiometal and, preferably, have N₂S₂ or N₃S cores. Exemplary NₓS_{y} chelates are described, e.g., in Fritzberg et al. (1988) PNAS 85:4024-29; and Weber et al. (1990) Bioconjugate Chem. 1:431-37; and in the references cited therein.

The Jacobsen et al. PCT application WO 98/12156 provides methods and compositions, i.e. synthetic libraries of binding moities, for identifying compounds which bind to a metal atom. The approach described in that publication can be used to identify binding moieties which can subsequently be added to ECBPs to derive the modified ECBPs of the present invention.

A problem frequently encountered with the use of conjugated proteins in radiotherapeutic and radiodiagnostic applications is a potentially dangerous accumulation of the radiolabeled moiety fragments in the kidney. When the conjugate is formed using a acid- or base-labile linker, cleavage of the radioactive chelate from the protein can advantageously occur. If the chelate is of relatively low molecular weight, as most of the subject modified ECBPs are expected to be, it is not retained in the kidney and is excreted in the urine, thereby reducing the exposure of the kidney to radioactivity. However, in certain instances, it may be advantageous to utilize acid- or base-labile linkers in the subject ligands for the same reasons they have been used in labeled proteins.

Accordingly, certain of the subject modified ECBPs can be synthesized, by standard methods known in the art, to provide reactive functional groups which can form acid-labile linkages with, e.g., a carbonyl group of the ligand. Examples of suitable acid-labile linkages include hydrazone and thiosemicarbazone functions. These are formed by reacting the oxidized carbohydrate with chelates bearing hydrazide, thiosemicarbazide, and thiocarbazide functions, respectively.

Alternatively, base-cleavable linkers, which have been used for the enhanced clearance of the radiolabel from the kidneys, can be used. See, for example, Weber et al. 1990 Bioconjug. Chem. 1:431. The coupling of a bifunctional chelate to an ECBP via a hydrazide linkage can incorporate base-sensitive ester moieties in a linker spacer arm. Such an ester-containing linker unit is exemplified by ethylene glycolbis (succinimidyl succinate), (EGS, available from Pierce Chemical Co., Rockford, Ill.), which has two terminal N-hydroxysuccinimide (NHS) ester derivatives of two 1,4-dibutyric acid units, each of which are linked to a single ethylene glycol moiety by two alkyl esters. One NHS ester may be replaced with a suitable amine-containing BFC (for example 2-aminobenzyl DTPA), while the other NTIS ester is reacted with a limiting amount of hydrazine. The resulting hyrazide is used for coupling to the ECBP, forming an ligand-BFC linkage containing two alkyl ester functions. Such a conjugate is stable at physiological pH, but readily cleaved at basic pH.

ECBPs labeled by chelation are subject to radiation-induced scission of the chelator and to loss of radioisotope by dissociation of the coordination complex. In some instances, metal dissociated from the complex can be re-complexed, providing more rapid clearance of non-specifically localized isotope and therefore less toxicity to non-target tissues. For example, chelator compounds such as EDTA or DTPA can be infused into patients to provide a pool of chelator to bind released radiometal and facilitate excretion of free radioisotope in the urine.

In still other embodiments, the peptide or peptidomimetic or fusion protein is coupled to a Boron addend, such as a carborane. For example, carboranes can be prepared with carboxyl functions on pendant side chains, as is well known in the art. Attachment of such carboranes to an amine functionality, e.g., as may be provided on the ECBP, can be achieved by activation of the carboxyl groups of the carboranes and condensation with the amine group to produce the conjugate. Such modified ECBPs can be used for neutron capture therapy.

The present invention also contemplates the modification of the subject peptides with dyes, for example, useful in photodynamic therapy, and used in conjunction with appropriate non-ionizing radiation. The use of light and porphyrins in methods of the present invention is also contemplated and their use in cancer therapy has been reviewed. van den Bergh, Chemistry in Britain, 22: 430-437 (1986), which is incorporated herein in its entirety by reference.

D. Nucleic Acid Compositions

In another aspect of the invention, the proteins described herein are provided in expression vectors. For instance, expression vectors are contemplated which include a nucleotide sequence encoding a polypeptide containing at least one ECBP sequence, which coding sequence is operably linked to at least one transcriptional regulatory sequence. Regulatory sequences for directing expression of the instant fusion proteins are art-recognized and are selected by a number of well understood criteria. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding the fusion proteins of this invention. Such useful expression control sequences, include, for example, the early and late promoters of SV40, adenovirus or cytomegalovirus immediate early promoter, the lac system, the trp system, the TAC or TRC system, T7 promoter whose expression is directed by T7 RNA polymerase, the promoter for 3-phosphoglycerate kinase or other glycolytic enzymes, the promoters of acid phosphatase, e.g., Pho5, and the promoters of the yeast α-mating factors and other sequences known to control the expression of genes of prokaryotic or eukaryotic cells or their viruses, and various combinations thereof. It should be understood that the design of the expression vector may depend on such factors as the choice of the host cell to be transformed. Moreover, the vector's copy number, the ability to control that copy number and the expression of any other protein encoded by the vector, such as antibiotic markers, should also be considered.

As will be apparent, the subject gene constructs can be used to cause expression of the subject fusion proteins in cells propagated in culture, e.g. to produce proteins or polypeptides, including fusion proteins, for purification.

This invention also pertains to a host cell transfected with a recombinant gene in order to express one of the subject polypeptides. The host cell may be any prokaryotic or eukaryotic cell. For example, a fusion proteins of the present invention may be expressed in bacterial cells such as E. coli, insect cells (baculovirus), yeast, or mammalian cells. Other suitable host cells are known to those skilled in the art.

Accordingly, the present invention further pertains to methods of producing the subject fusion proteins. For example, a host cell transfected with an expression vector encoding a protein of interest can be cultured under appropriate conditions to allow expression of the protein to occur. The protein may be secreted, by inclusion of a secretion signal sequence, and isolated from a mixture of cells and medium containing the protein. Alternatively, the protein may be retained cytoplasmically and the cells harvested, lysed and the protein isolated. A cell culture includes host cells, media and other byproducts. Suitable media for cell culture are well known in the art. The proteins can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the protein.

Thus, a coding sequence for a fusion protein of the present invention can be used to produce a recombinant form of the protein via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial cells), are standard procedures.

Expression vehicles for production of a recombinant protein include plasmids and other vectors. For instance, suitable vectors for the expression of the instant fusion proteins include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning: A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant fusion proteins by the use of a baculovirus expression system. Examples of such baculovirus expression systems include pVL-derived vectors (such as pVL1392, pVL1393 and pVL941), pAcUW-derived vectors (such as pAcUWI), and pBlueBac-derived vectors (such as the beta-gal containing pBlueBac III).

In yet other embodiments, the subject expression constructs are derived by insertion of the subject gene into viral vectors including recombinant retroviruses, adenovirus, adeno-associated virus, and herpes simplex virus-1, or recombinant bacterial or eukaryotic plasmids. As described in greater detail below, such embodiments of the subject expression constructs are specifically contemplated for use in various in vivo and ex vivo gene therapy protocols.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a fusion protein of the present invention, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.) Greene Publishing Associates, (1989), Sections 9.10-9.14 and other standard laboratory manuals. Examples of suitable retroviruses include pLJ, pZIP, pWE and pEM which are well known to those skilled in the art. Retroviruses have been used to introduce a variety of genes into many different cell types, including neural cells, epithelial cells, endothelial cells, lymphocytes, myoblasts, hepatocytes, bone marrow cells, in vitro and/or in vivo (see for example Eglitis et al., (1985) Science 230:1395-1398; Danos and Mulligan, (1988) PNAS USA 85:6460-6464; Wilson et al., (1988) PNAS USA 85:3014-3018; Armentano et al., (1990) PNAS USA 87:6141-6145; Huber et al., (1991) PNAS USA 88:8039-8043; Ferry et al., (1991) PNAS USA 88:8377-8381; Chowdhury et al., (1991) Science 254:1802-1805; van Beusechem et al., (1992) PNAS USA 89:7640-7644; Kay et al., (1992) Human Gene Therapy 3:641-647; Dai et al., (1992) PNAS USA 89:10892-10895; Hwu et al., (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234, WO94/06920, and WO94/11524). For instance, strategies for the modification of the infection spectrum of retroviral vectors include: coupling antibodies specific for cell surface antigens to the viral env protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral env proteins (Neda et al., (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

Another viral gene delivery system useful in the present invention utilizes adenovirus-derived vectors. The genome of an adenovirus can. be manipulated such that it encodes a gene product of interest, but is inactivate in terms of its ability to replicate in a normal lytic viral life cycle (see, for example, Berkner et al., (1988) BioTechniques 6:616; Rosenfeld et al., (1991) Science 252:431-434; and Rosenfeld et al., (1992) Cell 68:143-155). Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types, including airway epithelium (Rosenfeld et al., (1992) cited supra), endothelial cells (Lemarchand et al., (1992) PNAS USA 89:6482-6486), hepatocytes (Herz and Gerard, (1993) PNAS USA 90:2812-2816) and muscle cells (Quantin et al., (1992) PNAS USA 89:2581-2584). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al., (1979) Cell 16:683, Berkner et al., supra; and Graham et al., in Methods in Molecular Biology, E. J. Murray, Ed. (Humana, Clifton, N.J., 1991) vol. 7. pp. 109-127). Expression of the inserted chimeric gene can be under control of, for example, the E1A promoter, the major late promoter (MLP) and associated leader sequences, the viral E3 promoter, or exogenously added promoter sequences.

Yet another viral vector system useful for delivery of the subject chimeric genes is the adeno-associated virus (AAV). Adeno-associated virus is a naturally occurring defective virus that requires another virus, such as an adenovirus or a herpes virus, as a helper virus for efficient replication and a productive life cycle. (For a review, see Muzyczka et al., Curr. Topics in Micro. and Immunol. (1992) 158:97-129). It is also one of the few viruses that may integrate its DNA into non-dividing cells, and exhibits a high frequency of stable integration (see for example Flotte et al., (1992) Am. J. Respir. Cell. Mol. Biol. 7:349-356; Samulski et al., (1989) J. Virol. 63:3822-3828; and McLaughlin et al., (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al., (1985) Mol. Cell. Biol. 5:3251-3260 can be used to introduce DNA into cells. A variety of nucleic acids have been introduced into different cell types using AAV vectors (see for example Hermonat et al., (1984) PNAS USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

Other viral vector systems that may have application in gene therapy have been derived from herpes virus, vaccinia virus, and several RNA viruses. In particular, herpes virus vectors may provide a unique strategy for persistence of the recombinant gene in cells of the central nervous system and ocular tissue (Pepose et al., (1994) Invest Ophthalmol Vis Sci 35:2662-2666)

In addition to viral transfer methods, such as those illustrated above, non-viral methods can also be employed to cause expression of a protein in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the gene by the targeted cell. Exemplary gene delivery systems of this type include liposomal derived systems, poly-lysine conjugates, and artificial viral envelopes.

In a representative embodiment, a gene encoding an ECBP-containing polypeptide can be entrapped in liposomes bearing positive charges on their surface (e.g., lipofectins) and (optionally) which are tagged with antibodies against cell surface antigens of the target tissue (Mizuno et al., (1992) No Shinkei Geka 20:547-551; PCT publication WO91/06309; Japanese patent application 1047381; and European patent publication EP-A-43075). For example, lipofection of neuroglioma cells can be carried out using liposomes tagged with monoclonal antibodies against glioma-associated antigen (Mizuno et al., (1992) Neurol. Med. Chir. 32:873-876).

In yet another illustrative embodiment, the gene delivery system comprises an antibody or cell surface ligand which is cross-linked with a gene binding agent such as poly-lysine (see, for example, PCT publications WO93/04701, WO92/22635, WO92/20316, WO92/19749, and WO92/06180). For example, any of the subject gene constructs can be used to transfect specific cells in vivo using a soluble polynucleotide carrier comprising an antibody conjugated to a polycation, e.g., poly-lysine (see U.S. Pat. No. 5,166,320). It will also be appreciated that effective delivery of the subject nucleic acid constructs via -mediated endocytosis can be improved using agents which enhance escape of the gene from the endosomal structures. For instance, whole adenovirus or fusogenic peptides of the influenza HA gene product can be used as part of the delivery system to induce efficient disruption of DNA-containing endosomes (Mulligan et al., (1993) Science 260-926; Wagner et al., (1992) PNAS USA 89:7934; and Christiano et al., (1993) PNAS USA 90:2122).

In clinical settings, the gene delivery systems can be introduced into a patient by any of a number of methods, each of which is familiar in the art.

For instance, a pharmaceutical preparation of the gene delivery system can be introduced systemically, e.g., by intravenous injection, and specific transduction of the construct in the target cells occurs predominantly from specificity of transfection provided by the gene delivery vehicle, cell-type or tissue-type expression due to the transcriptional regulatory sequences controlling expression of the gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al., (1994) PNAS USA 91: 3054-3057).

E. Exemplary Formulations

The subject compositions may be used alone, or as part of a conjoint therapy with other chemotherapeutic compounds.

The ECBP therapeutics for use in the subject method may be conveniently formulated for administration with a biologically acceptable medium, such as water, buffered saline, polyol (for example, glycerol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the ECBP therapeutics, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., U.S.A. 1985). These vehicles include injectable “deposit formulations”.

Pharmaceutical formulations of the present invention can also include veterinary compositions, e.g., pharmaceutical preparations of the ECBP therapeutics suitable for veterinary uses, e.g., for the treatment of live stock or domestic animals, e.g., dogs, cats and like.

Other formulations of the present invention include agricultural formulations, e.g., for application to plants.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of an ECBP therapeutic at a particular target site.

The pharmaceutical compositions according to the present invention may be administered as either a single dose or in multiple doses. The pharmaceutical compositions of the present invention may be administered either as individual therapeutic agents or in combination with other therapeutic agents. The treatments of the present invention may be combined with conventional therapies, which may be administered sequentially or simultaneously. The pharmaceutical compositions of the present invention may be administered by any means that enables the ECBP moiety to reach the targeted cells. In some embodiments, routes of administration include those selected from the group consisting of oral, intravesically, intravenous, intraarterial, intraperitoneal, local administration into the blood supply of the organ in which the tumor resides or directly into the tumor itself. Intravenous administration is the preferred mode of administration. It may be accomplished with the aid of an infusion pump.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other mammals for therapy by any suitable route of administration, including orally, intravesically, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular ECBP therapeutic employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient will range from about 0.0001 to about 100 mg per kilogram of body weight per day.

Because the subject ligands are specifically targeted to tumor endothelial cells, those modified ECBPs which comprise chemotherapeutics or toxins can be administered in doses less than those which are used when the chemotherapeutics or toxins are administered as unconjugated active agents, preferably in doses that contain up to 100 times less active agent. In some embodiments, modified ECBPs which comprise chemotherapeutics or toxins are administered in doses that contain 10-100 times less active agent as an active moiety than the dosage of chemotherapeutics or toxins administered as unconjugated active agents. To determine the appropriate dose, the amount of compound is preferably measured in moles instead of by weight. In that way, the variable weight of different modified ECBPs does not affect the calculation. Presuming a one to one ratio of modified ECBP to active moiety in modified ECBPs of the invention, less moles of modified ECBPs may be administered as compared to the moles of unmodified ECBPs administered, preferably up to 100 times less moles.

If desired, the effective daily dose of the active compound may be administered as two, three, four, five, six or more sub-doses administered separately at appropriate intervals throughout the day, optionally, in unit dosage forms.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable carriers and can also be administered in conjunction with other antimicrobial agents such as penicillins, cephalosporins, aminoglycosides and glycopeptides. Conjunctive therapy, thus includes sequential, simultaneous and separate administration of the active compound in a way that the therapeutical effects of the first administered one is not entirely disappeared when the subsequent is administered.

### V. Examples

A. Binding of Synthetic Peptides to Endothelial Cells

We developed a cell-based saturation ligand binding assay to measure the specific binding of the isolated EC binding peptides to human microvascular endothelial cells (HMVEC) and aortic smooth muscle cells (AoSMC). This assay allows the determination of the relative binding affinities of the isolated EC binding peptides. The assay is based on the highly sensitive fluorescence enhancement technique DELFIA® and coated microtitration plates. In this assay the synthetic peptides labeled with biotin at their amino terminus are incubated with fixed target cells in microtiter plate format. After incubation, the excess, unbound peptides are removed and europium labeled streptavidin added to the plates. After the removal of excess streptavidin, the europium is released and its fluorescence is measured.

Saturation ligand binding experiments measure specific ligand binding at equilibrium at various concentrations of the ligand. Binding curves are analyzed to determine binding site or receptor number and affinity. This curve is known as a rectangular hyperbola, binding isotherm, or saturation binding curve. This curve is determined by the following equation:

Y=(Bₘₐₓ*X)/(K_{d}+X)

Y is zero initially, and increases to a maximum plateau value, Bmax. This equation describes the equilibrium binding of a ligand to a receptor as a function of increasing ligand concentration. X is the concentration of the ligand, and Y is the specific binding. K_{d} is the equilibrium dissociation constant, expressed in the same units as the X-axis (concentration). When the drug concentration equals K_{d}, half the binding sites are occupied at equilibrium.

(i) Chemicals, Supplies and Equipment

96-well microtiter plates (Corning, N.Y., cat # 3997) and reagent reservoir (Corning, N.Y., cat # 4870) were purchased from Corning, Inc. Cells were dispensed into the 96-well plates with Multidrop and plates were washed with Titertek automated plate washer (ICN-Titertek). 4% buffered formalin was purchased from Sigma Diagnostics (St. Louis, Mo., cat. # HT50-1-2). Pierce Casein Blocker was bought from Pierce (Rockford, Ill., cat # 37532). Custom biotinylated peptides were synthesized by Bio-Synthesis, Inc. (Lewisville, Tex.).

Streptavidin-Eu³⁺ reagent (cat # 1244-105), reagent diluent (cat # 1244-111), and Enhancement Solution (cat # 1244-105) were purchased from Perkin Elmer-Wallac (Turku, Finland), and the plates were read on Wallac Victor counter. The binding constants were calculated using the method of nonlinear regression analysis with the Prizm software (GraphPad Software, Inc.).

(ii) Cell Culture

The HMVEC (human microvascular endothelial cell) and aortic smooth muscle cells (AoSMC) cells were purchased from Clonetics (San Diego, Calif.) and maintained according to the specifications of the supplier. In these experiments the cultures were used from passages 5 and 6 only.

(iii) Saturation Binding Assay

The cultured subconfluent cells were trypsinized and resuspended in the culture media according to Clonetics instructions. The diluted suspension was dispensed into a reagent reservoir with magnetic stirring bar. 50 μl of the cell suspension was dispensed into each well of the 96-well plate with the Multidrop while stirring the suspension. The seeding density was 5*10³ cells/well. The plates are incubated at 37° C. in the atmosphere containing 5% CO₂ for 48 hours.

The cells were then washed three times with TBS with 0.01%Tween20 (wash buffer) and fixed with 2% formalin solution for fifteen minutes at 20° C. The cells were subsequently washed three times with wash buffer. The nonspecific sites on the plates were blocked by 300 μl of Pierce Casein Blocker per well for two hours at 20° C.

The saturation binding assay was performed by adding 50μl of the biotinylated peptide per well at increasing concentrations. The concentration was ranging from 1 nM to 10 μM. All measurements were performed in triplicates. Peptide dilutions were prepared from 10 mM DMSO stocks in wash buffer. The plates were incubated for one hour at room temperature and subsequently washed five times with wash buffer using Titertek automatic plate washer to remove any unbound peptide. The biotinylated peptide bound to the cells was then allowed to react for thirty minutes with 50 μl of 2000× dilution of the streptavidin-Eu³⁺ reagent, diluted in the Wallac reagent diluent. Finally, after washing the unbound streptavidin-Eu³⁺ reagent, 50 μl of Wallac Enhancement Solution per well was added to release the free Eu³⁺ from its chelate form. Free Eu³⁺ rapidly forms a highly fluorescent stable chelate with the components of the Enhancement solution. The fluorescence (λₑₓ=340 nm, λₑₘ=613 nm) was then measured with time-resolved fluorometer Victor I.

The specificity of binding of the peptides to the cells was determined by performing the binding to the blocked wells of the microtiter plates. The binding under the assay conditions was negligible under 10 μM peptide concentration. The nonspecific binding of the streptavidin-Eu³⁺ reagent to the cells was determined by omitting the biotinylated peptide incubation step in the assay. There was no appreciable binding of streptavidin-Eu³⁺ reagent to the cells in the assay.

(iv) Final Assay Conditions:

Total Reaction Volume=50 μL

Final DMSO Concentration Maximum 0.5%

Blank Condition: Peptide Solution Diluent Added to the Cells

Positive Control: RDG Peptide

Negative Control: Unrelated Peptide

(v) Results

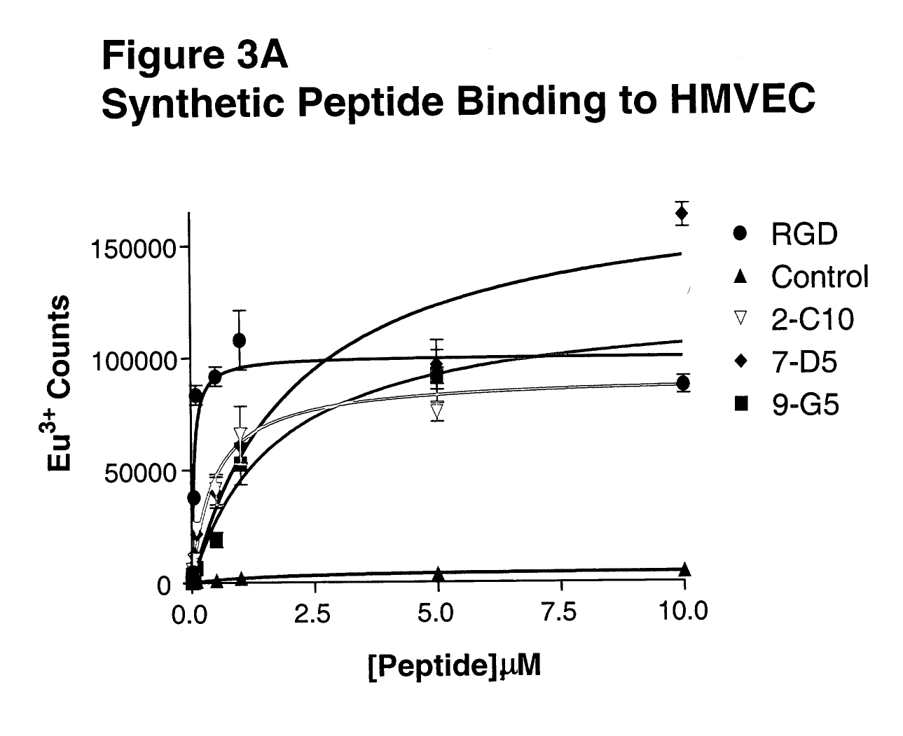

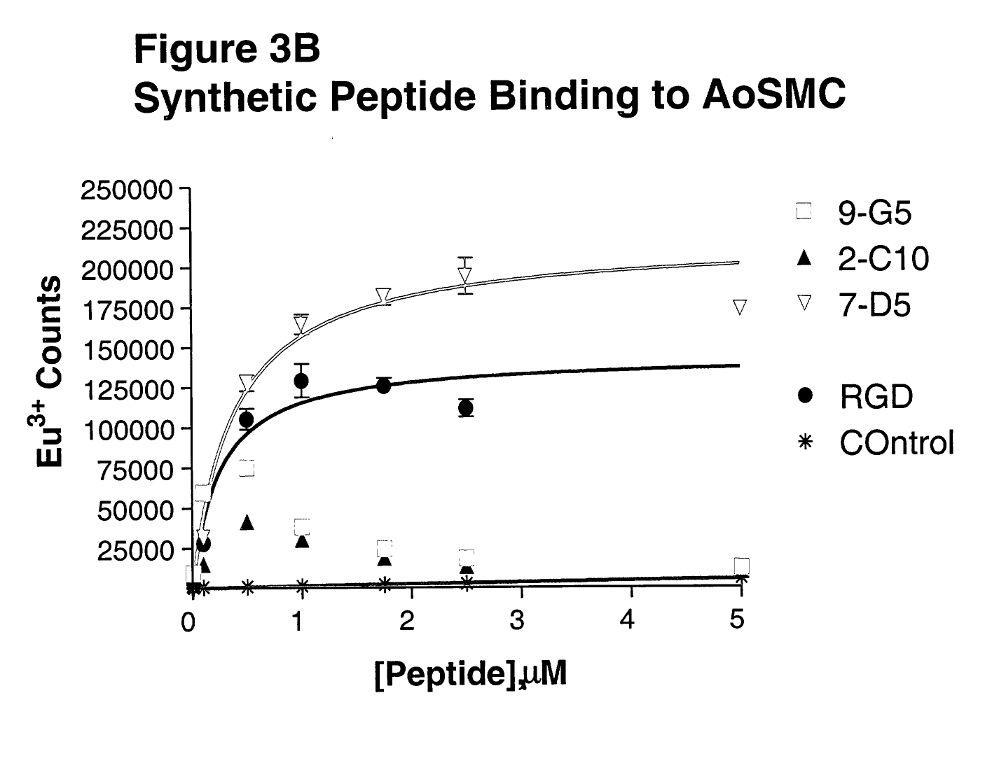

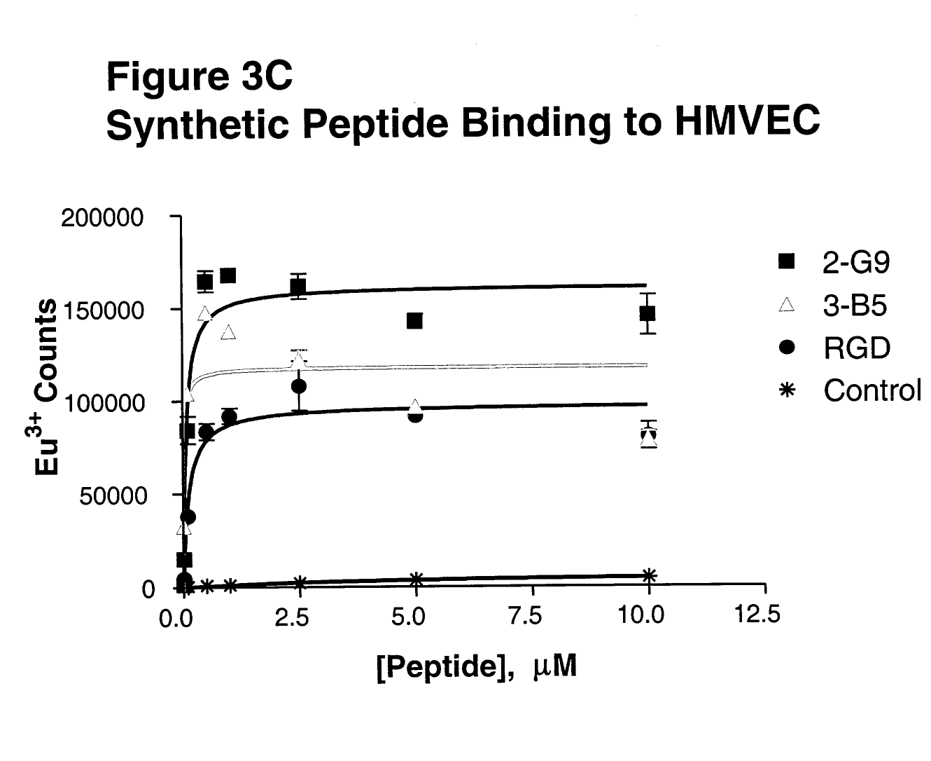

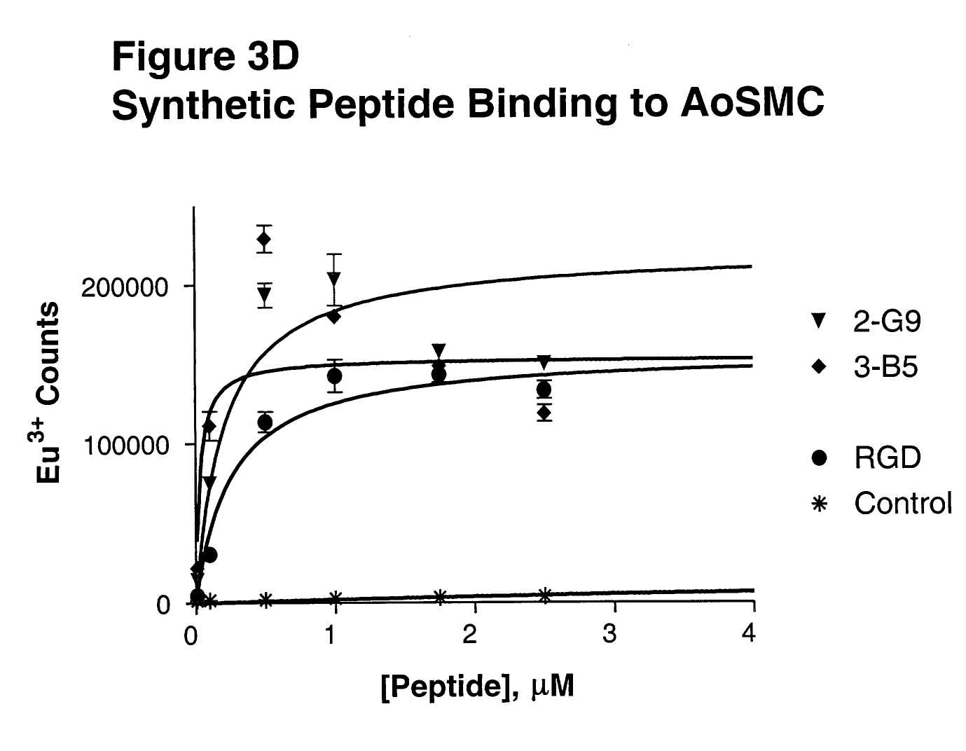

FIGS. 3A-D represents saturation binding data from typical binding experiments. The binding parameters are summarized as follows.

| | Cell | | Bmax | Binding |
|---|---|---|---|---|
| Peptide Name | Type | Kd (μM) | (counts/cell) | Affinity |
| 2-C10 (SEQ ID NO: 89) | AoSMC | −0.1698 | 0.9694 | −5.709 |
| 2-G9 (SEQ ID NO: 82) | AoSMC | 0.1478 | 8.519 | 57.639 |
| 3-B5 (SEQ ID NO:) | AoSMC | 0.098 | 8.354 | 85.24 |
| 7-D5 (SEQ ID NO: 251) | AoSMC | 0.384 | 10.8332 | 28.211 |
| 9-G5 (SEQ ID NO: 301) | AoSMC | −1.381 | 2.937 | −2.127 |
| RGD (SEQ ID NO: 685) | AoSMC | 0.2501 | 7.18 | 28.725 |
| 2-C10 (SEQ ID NO: 89) | HMVEC | 2.293 | 8.79 | 3.83 |
| 2-G9 (SEQ ID NO: 82) | HMVEC | 0.031 | 5.15 | 166.12 |
| 3-B5 (SEQ ID NO:) | HMVEC | 0.0097 | 3.817 | 394.75 |
| 9-G5 (SEQ ID NO: 301) | HMVEC | 0.527 | 10.1138 | 19.195 |
| 7-D5 (SEQ ID NO: 251) | HMVEC | 4.768 | 11.0211 | 2.3116 |

The peptide sequences are:

Control peptides:

| | | |
|---|---|---|
| RGD: | Biotin-CDCRGDCFC-OH | (SEQ ID NO: 685, |
| | | Positive control) |
| Control: | Biotin-HIPRSPYKF-OH | (SEQ ID NO: 686, |
| | | Negative control) |

EC binding peptides:

| | | |
|---|---|---|
| 2-C10: | Biotin-GCHSSTWRACG | (SEQ ID NO: 89) |
| 2-G9: | Biotin-GCPTPHSGTCG | (SEQ ID NO: 82) |
| 3-B5: | Biotin-GCMNQHSSACG | (SEQ ID NO: ) |
| 7-D5: | Biotin-GCTQMRTAYCG | (SEQ ID NO: 251) |
| 9-G5: | Biotin-GCDSHKRLKCG | (SEQ ID NO: 301) |

(vi) Conclusions

In these binding experiments we observed strong, specific binding of all the EC binding peptides to HMVEC cells (FIGS. 3A, B). Interestingly, peptides 2-C10 and 9-G5 do not bind to AoSMC while peptide 7-D5 binds better to smooth muscle cells than to endothelial cells.

B. Inhibition of EC Proliferation by Synthetic EC Binding Peptides

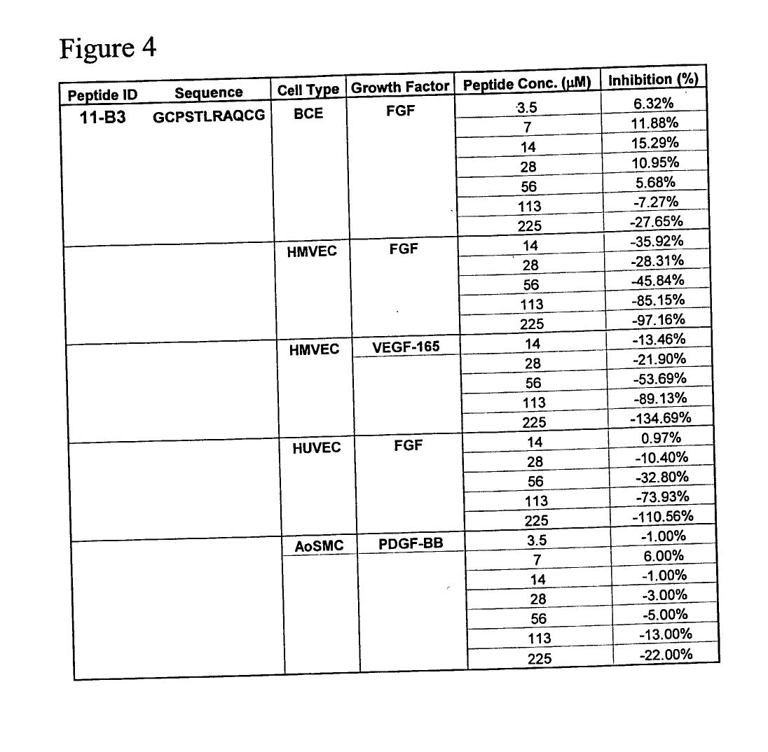

We performed proliferation assays to determine the effect of the synthetic EC binding peptides on the proliferation of endothelial cells. In these assays the proliferation of bovine capillary endothelial (BCE), human microvascular endothelial (HMVEC) and human vascular endothelial cells (HUVEC) was stimulated by FGF and VEGF₁₆₅ as indicated on FIG. 4. The effect of increasing concentration of synthetic EC binding peptides was measured on the proliferation of the stimulated cells. After the 72 h incubation the media was removed, the plates were washed two times with PBS and frozen at −80° C. Proliferation of the BCE cells was accessed using the CyQUANT® fluorescent cell proliferation assay kit according to the manufacturer's recommendations.

The effect of the synthetic peptides on the proliferation of normal human dermal fibroblasts (NHDF) and aortic smooth muscle cells (AoSMC) was measured to assess their EC specific inhibitory activity. The proliferation of NHDF was stimulated either by FGF or PDGF-BB. The proliferation of AoSMC was stimulated by PDGF-BB.

FIG. 4. shows the peptide ID and sequence, the inhibitory effect of the indicated concentration of the peptide on the target cell and the growth factor used to stimulate proliferation. The inhibition was calculated as the percentage of peptide inhibition compared to the non-stimulated control, which was 100%.

C. Inhibition of EC Proliferation by Serum Albumin-EC Binding Peptide Fusions

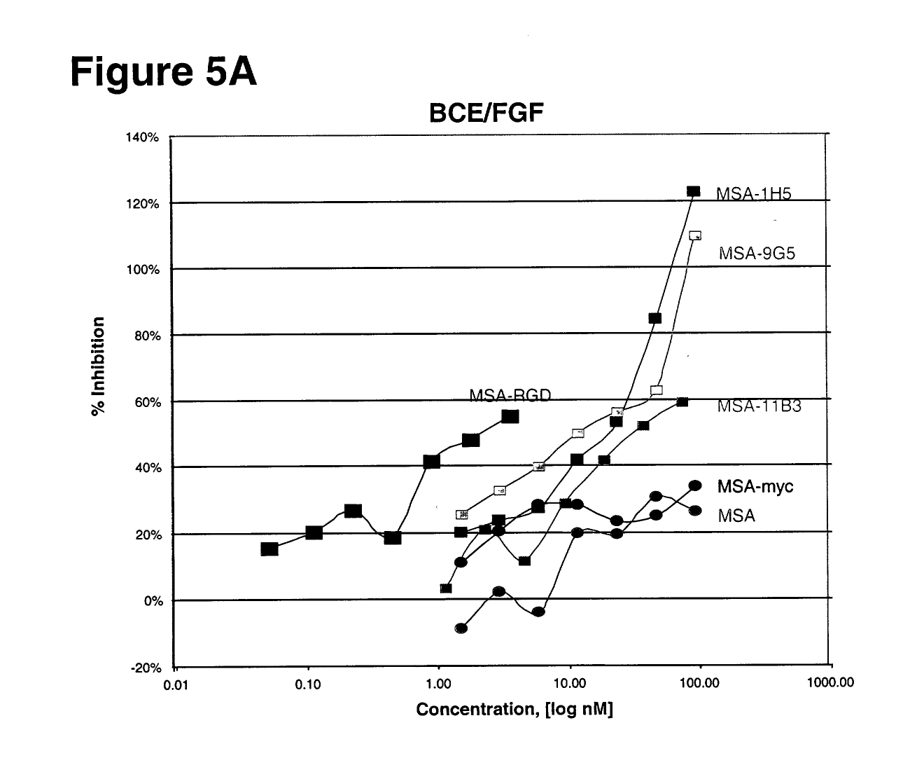

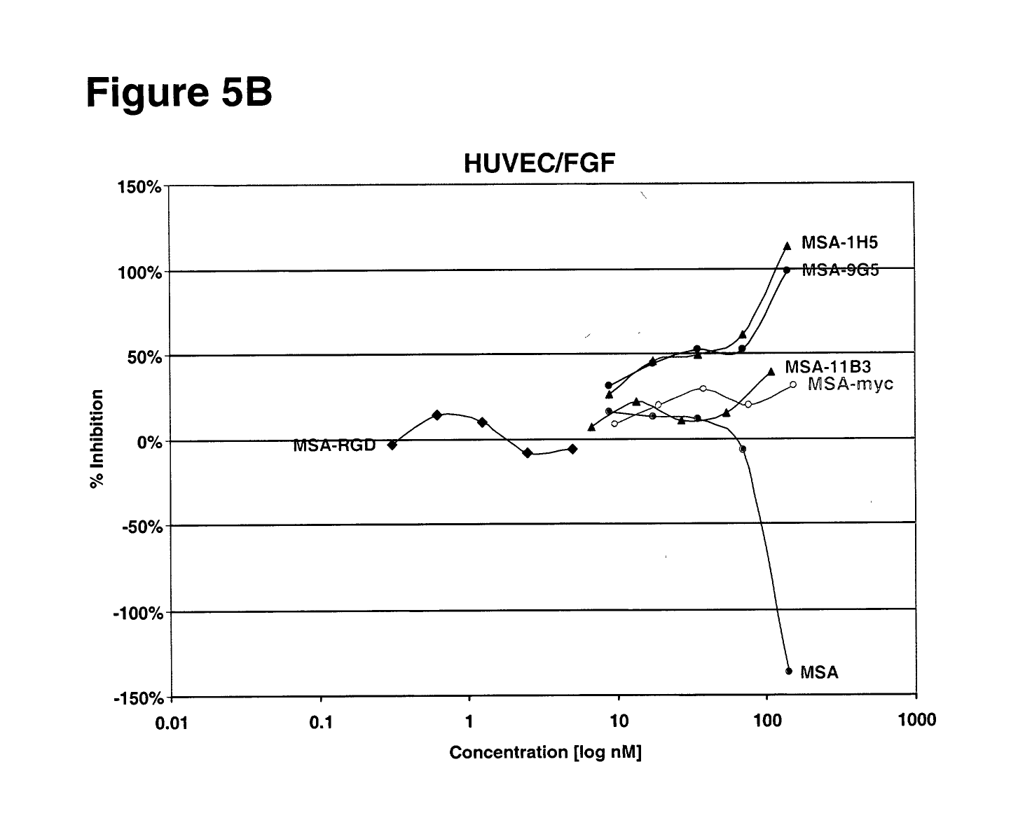

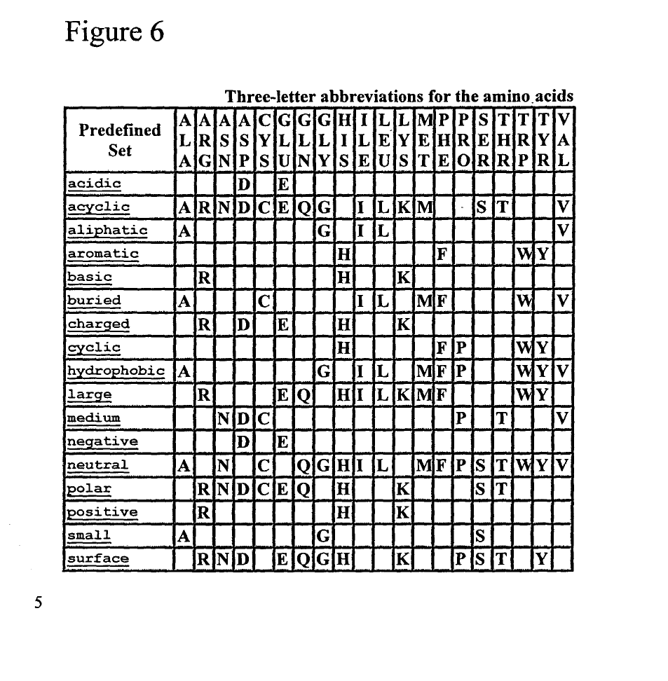

This experiment was designed to demonstrate the inhibition of BCE and HUVEC cell proliferation by purified mouse serum albumin (MSA) proteins that displayed endothelial cell binding (EC) peptides. In the MSA-peptide fusions the peptide sequence was inserted into a cysteine constrained loop between amino acids 53 and 62. The proteins were produced by COS-7 cells that were transfected with expression plasmids that directed the synthesis and secretion of the particular recombinant protein. As it is shown on FIG. 5A, in the MSA-9G5, MSA-11B3 and MSA-RGD constructs the inserted peptides substituted the naturally occurring residues of MSA between cys⁵³-cys⁶². In MSA-1H5 and MSA-myc constructs (negative control) the peptides were inserted into the loop at amino acid glu⁵⁷ (FIG. 5A). FIG. 5B and FIG. 5C shows the inhibitory effect of the purified proteins on the proliferation of BCE and HUVEC cells that were stimulated by FGF.

(i) Experimental Design of the EC Proliferation Experiments

Protein Production and Concentration:

COS7-L cells were transfected with protein expression constructs expressing:

1. MSA, full-length mouse serum albumin (negative control)

2. MSA-RGD, in which the RGD sequence (VRGDF, SEQ ID NO: 687) replaces the MSA sequence between Cys 53 and Cys 62

3. MSA-11B3, in which the 11-B3 peptide sequence (PSTLRAQ, SEQ ID NO: ) replaces the MSA sequence between Cys 53 and Cys 62

4. MSA-1H5, in which the 1-H5 peptide sequence (HTKQIPRHIYSA, SEQ ID NO: 54) is inserted between Glu 57 and Ser 58 within the Cys 53 and Cys 62 loop of MSA

5. MSA-9G5, in which the 9-G5 peptide sequence (DSHKRLK, SEQ ID NO: 301) replaces the MSA sequence between Cys 53 and Cys 62

6. MSA-myc, in which the Myc epitope peptide sequence (EQKLISEEDL, SEQ ID NO: 687) is inserted between Glu 57 and Ser 58 within the Cys 53 and Cys 62 loop of MSA (negative control)

The transfected COS7-L cells were cultured in defined serum free media (VP-SFM). Each day for 5 days the conditioned media was collected from the cells, centrifuged to remove dead cells and other cellular debris and then frozen. The 5 days worth of cultured media were pooled and concentrated 500-fold using a Centiprep-80 with a molecular weight cut-off of 50 (for MSA, MSA-RGD, MSA-9G5) or a molecular weight cut-off of 30 (for MSA-myc, MSA-11B3, MSA-1H5). The concentration of the albumin proteins was determined by Western blot analysis of each preparation using a rabbit anti-MSA antibody and using purified MSA of known concentration to generate a standard curve. Following development of the blot and exposure to film the autoradiographs were analyzed using the Gel Doc 1000 image analysis system and Molecular Analyst software (BioRad).

(ii) BCE Proliferation Assays

On day zero, bovine capillary endothelial cells (BCE) at passage 11 were plated in 96 well tissue culture plates at a density of 2×10³ cells per well in 100 μl 5% calf serum (CS)/DMEM supplemented with penicillin/streptomycin (PS). The cells were then incubated overnight in an atmosphere of 10% CO₂, 37° C.

On day one, the media was changed to 150 μl 2% CS/DMEM/PS. The albumin proteins were added to the first well as 8.75 μl which contains an additional 150 μl of 2% CS/DMEM/PS. 150 μl was then removed from this well and added to the next well resulting in a 1:2 dilution of the protein. This process was repeated for a total of six times each in triplicate. 50 μl of 4 ng/ml FGF (final concentration: 1 ng/ml FGF) was then added to each well and the plates incubated as above for 72 h. A synthetic peptide of cyclic RGD (c-RGD) at a concentration of 4.1 μM was included to serve as a positive control for inhibition of proliferation. Cells without addition of protein but with FGF added and without FGF added were included on each plate as additional controls.

After the 72 h incubation the media was removed, the plated washed two times with PBS and frozen at −80° C. Proliferation of the BCE cells was accessed using the CyQUANT® cell proliferation assay kit according to the manufacturer's recommendations.

(iii) Conclusions

The insertion of the EC binding peptides into MSA increased their inhibitory activity by approximately 1000-fold. The MSA-EC binding peptide fusions inhibited BCE and HUVEC proliferation in the nanomolar (nM) range while the synthetic peptides were active in the micromolar (μM) range. The control MSA and MSA-myc proteins did not significantly affect the proliferation of the target endothelial cells.

D. Inhibition of Angiogenesis by EC-Binding Peptides using Aortic Ring Assay

This experiment was designed to demonstrate the inhibition of angiogenesis by certain identified EC-binding peptides in a well-established angiogenesis assay.

The aortic ring assay is a widely used in vitro assay for angiogenesis. Typically, rat aortas are sliced crosswise and placed in Matrigel. After three to five days, microvessel sprouting can be roughly quantified by manual counting or by using image analysis. The aortic ring assay is low-cost, easy to use, and offers many assays from a single animal. A relatively new variant uses embryonic chick aortic arches (see Muthukkaruppan et al., Proceedings of the American Association for Cancer Research Annual Meeting, 2000).

In this experiment, chicken aortic rings are embedded in Matrigel (BD BioCoat™ Matrigel™ or other equivalent products) in optimized MCDB-131 media (Sigma product number: M8537, or equivalent products from other vendors). From endothelial cells (EC) in the aortic intima, a complex network of branching and anastomosing microvessels develop interspersed with fibroblast as individual cells. In this assay the ability of the identified EC-binding peptides is tested for their abilities to inhibit EC branching and microvessel formation.

Chicken aortic branches were dissected and cleaned from 15 day old chicken embryos, and were then rinsed well in cold PBS+Penicillin/Streptomycin (P/S) buffers. The arteries were then cut into about 1 mm rings using sterile razor blade, and the resulting rings were mounted in 10-20 μl of Matrigel (in a 48-well plate). Subsequently, 300 μl of MCDB-131 basal media+P/S containing the indicated amount of test peptide were added. The cultured rings were incubated at 37° C., with 5% CO₂ for 2-3 days, and the extent of microvessel formation were evaluated as the end of the experiment.

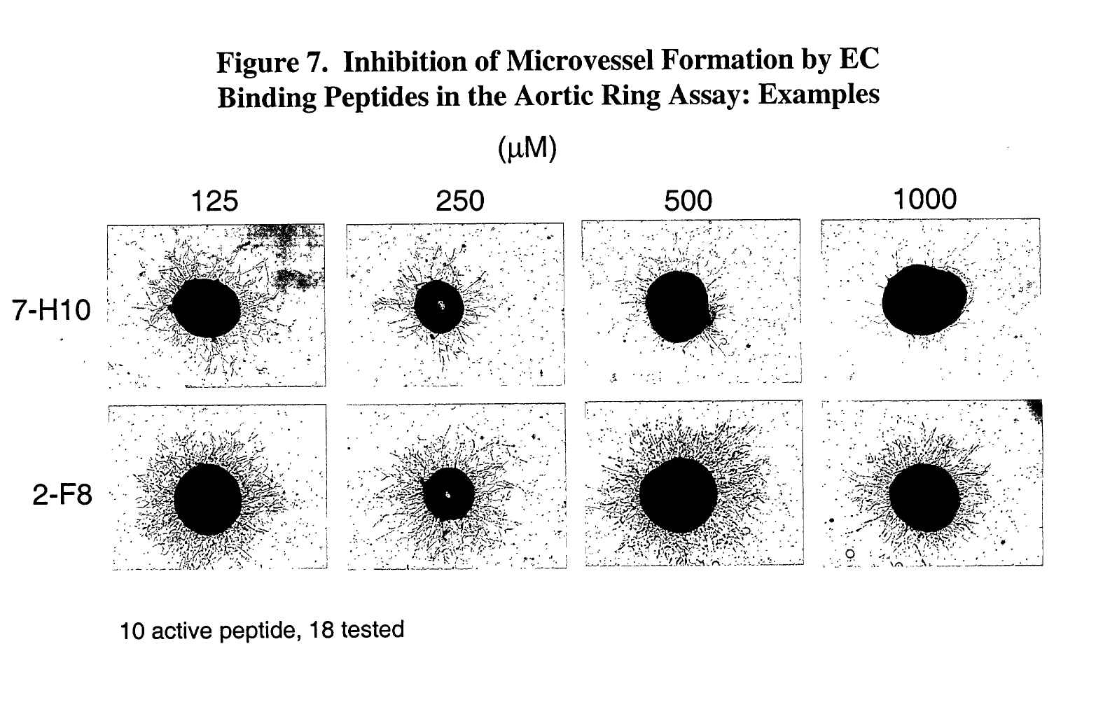

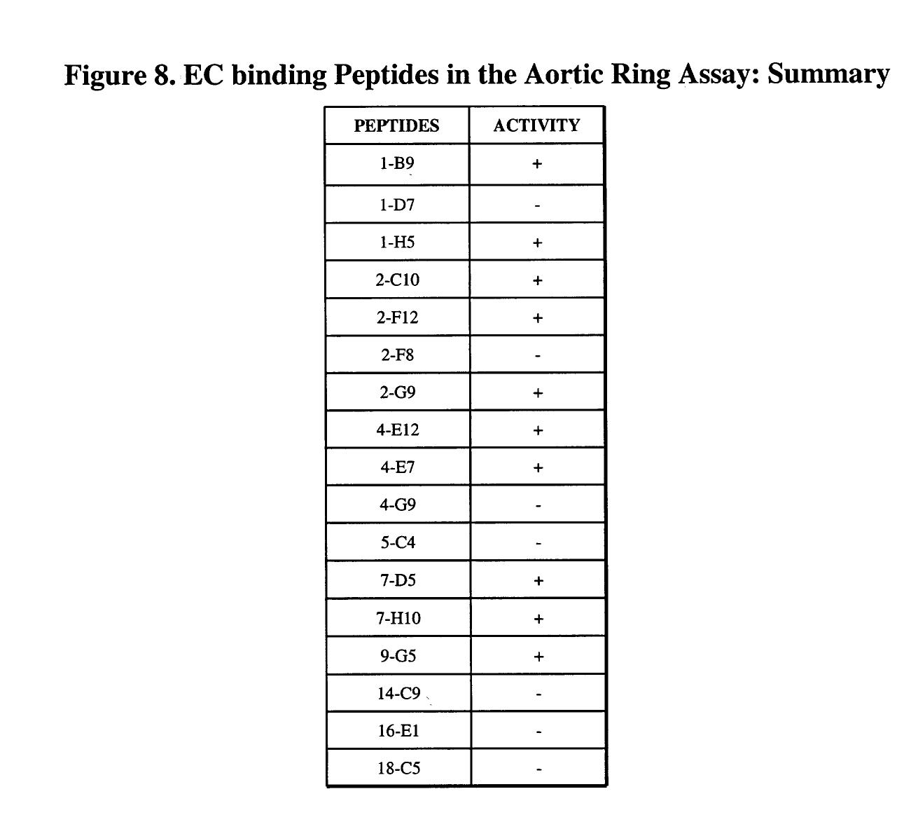

Eighteen polypeptides were tested in the aortic ring assay, 10 of which were found to be able to inhibit angiogenesis under the experimental conditions (see FIG. 8). FIG. 7 shows the results from two of the 18 tested peptides. Peptide 7-H10 (top panels) clearly inhibits angiogenesis in a dose dependent manner, while 2-F8 (bottom panels) does not appear to have an effect at the highest concentration (1 mM) tested.

E. Inhibition of Angiogenesis in vivo by EC-Binding Peptides using Matrigel Plug Assay (1 Week Assay)

This assay takes advantage of the fact that Matrigel is liquid at 4° C. but becomes gel when warmed to 37° C. Thus when injected subcutaneously into the ventral region of a mouse, Matrigel solidifies and forms a so-called “Matrigel plug”. When angiogenic factors, such as FGF, are mixed with the Matrigel and injected, endothelial cells migrate into the gel plug and form functional vessels. The level of angiogenesis can then be assessed by measuring hemoglobin levels in the plugs, which are directly proportional to the extent of angiogenesis. Typically, two plugs are assayed in each mouse: one plug (control plug) contains FGF only and is used as control for angiogenesis, while the test plug contains FGF and an indicated amount of test peptide(s). In single-point assays, peptide concentration in the plug is 1 mM.

Experimental Animals:

C57BL/6 female mice, 6-8 wks.

Matrigel Preparation:

Basis FGF (bFGF) stock was prepared at 25 μg/ml in saline buffer +5% mouse serum albumin. The bFGF stock was added to Matrigel at 200 ng/ml. The mixture was left on ice for at least 3 hours before further use. For each mouse, 1 ml of such mixture was prepared, so that 0.5 ml of the mixture can be injected into each mouse to achieve about 100 ng/one side injection. If a test peptide were also to be added, for each mouse, 50 μl of 10 mM peptide (or 5 mM cRGD, positive control) was slowly added to Matrigel, swirling while adding. Per 5 mice, add 300 μl of peptide per 3 mls of Matrigel.

Injection:

Syringes for injection were left on ice before injection. One syringe is used for each side of the group of 5 mice. About 1 ml of the mixture can be loaded at a time and injected into 2 mice using the Becton Dickinson 1 ml syringe for subcutaneous injection (26 gauge, ⅝ths, cat# 309597). Mice were anesthetized with isofluorane in a chamber, and were pulled out just as their breathing became slow. Injection were performed subcutaneously at inner thighs of the mice.

Measurement of Angiogenesis:

To determined the degree of angiogenesis, Matrigel plugs were dissected out, and placed in pre-weighed Falcon 5 ml snap-cap round bottomed tubes (polypropylene, Falcon cat# 352063). One ml of PBS was then added. The tubes were weighed with Matrigel plugs inside. The samples were kept at 4° C. until ready for hemoglobin assay.

Hemoglobin Assay:

Sonicate samples before measuring the hemoglobin content using the Plasma hemoglobin kit from Sigma.

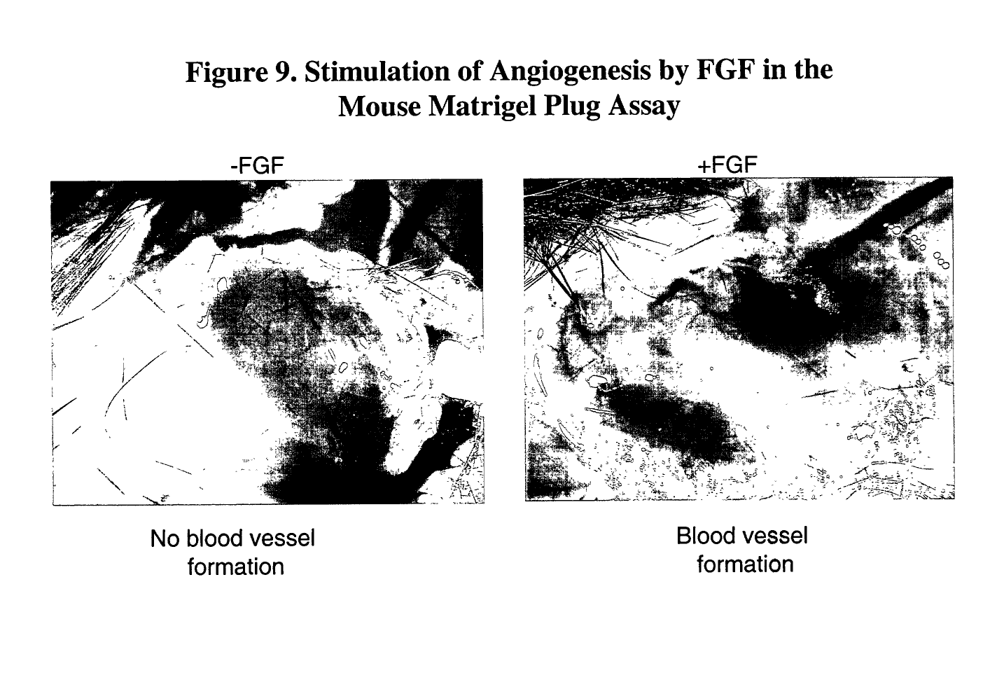

FIG. 9 shows an example of FGF-dependent angiogenesis in vivo in the injected Matrigel plugs. It is evident that blood vessel formation occurred when FGF is present in the Matrigel (right panel), while angiogenesis does not occur at the absence of FGF (left panel).

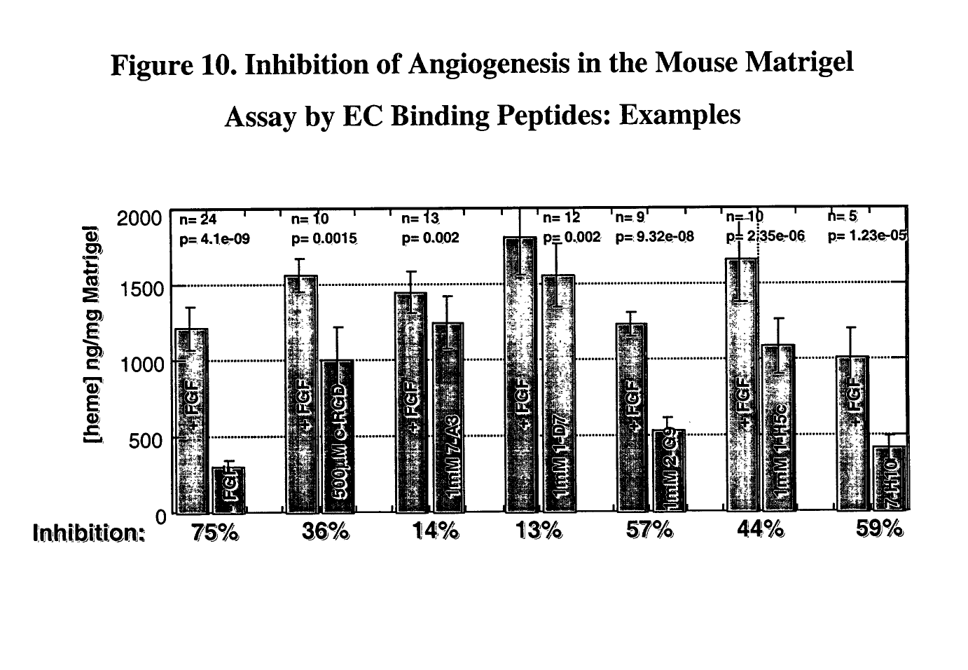

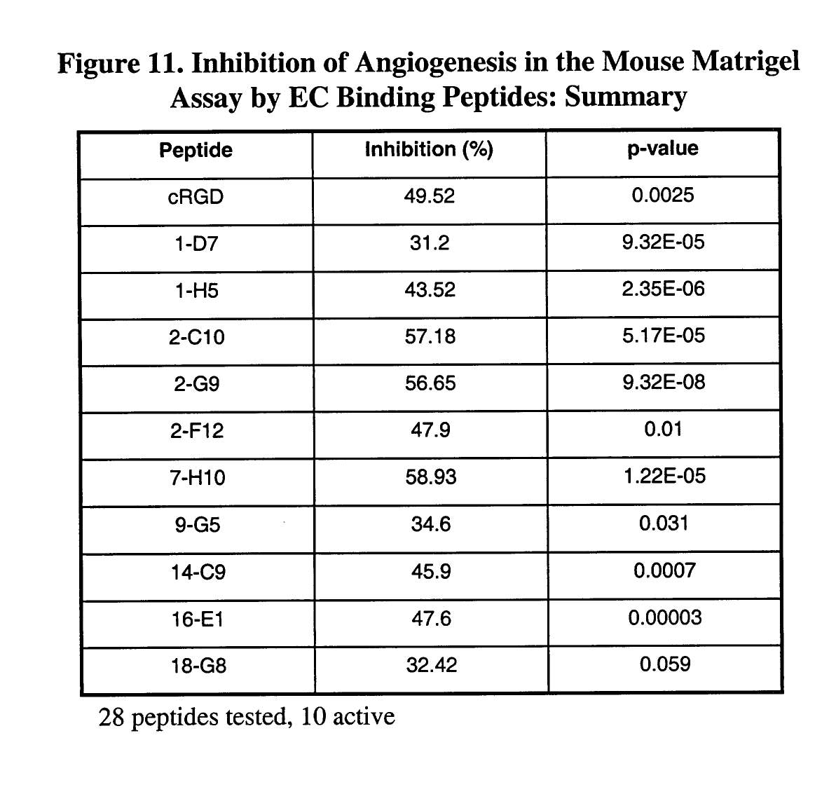

When the same experimental system was used to assess the angiogenesis inhibitory effects of certain identified EC-binding polypeptides, it was evident that certain peptides strongly inhibited angiogenesis in this in vivo model, some even stronger than the positive control cRGD. In FIG. 10, leaving out FGF is equivalent to a 75% inhibition of angiogenesis (see the left most two bars). The positive control cRGD can inhibit 36% of the observed angiogenesis at the presence of FGF (compare the second set of bars). Three peptides, 2-G9, 1-H5 and 7-H10, all exhibited stronger inhibitory effects that cRGD (57%, 44%, and 59% inhibition, respectively); while two peptides, 7-A3 and 1-D7, exhibited weaker yet statistically significant inhibitions (p<0.002). The results of the other tested peptides in this in vivo model are listed in FIG. 11. Collectively, 10 of the 28 peptides tested positively inhibited angiogenesis in this in vivo model.

It should be understood that each assay has its unique advantages and disadvantages. Therefore, certain peptides may test positive (for an angiogenesis inhibitory effect) in one assay but turns out to be negative in a different assay. However, as long as a peptide can be tested as positive in at least one of the commonly accepted methods in the art, a peptide is considered to be an angiogenesis inhibitor. If a peptide is tested positive in more than one assays, then it is more likely that such a peptide is an angiogenesis inhibitor.

Based on the aortic ring assay and the Matrigel plug assay, it can be concluded that at least 6 of the identified peptides (1-H5, 2-C10, 2-G9, 2-F12, 7-H10, and 9-G5, SEQ ID NOs. 54, 89, 82, 87, 253, and 301, respectively) tested positive in both assays, while at least three peptides (14-C9, 1-D7 and 16-E1, SEQ ID NOs. 442, 57, and 467, respectively) tested positive in one of the two assays.

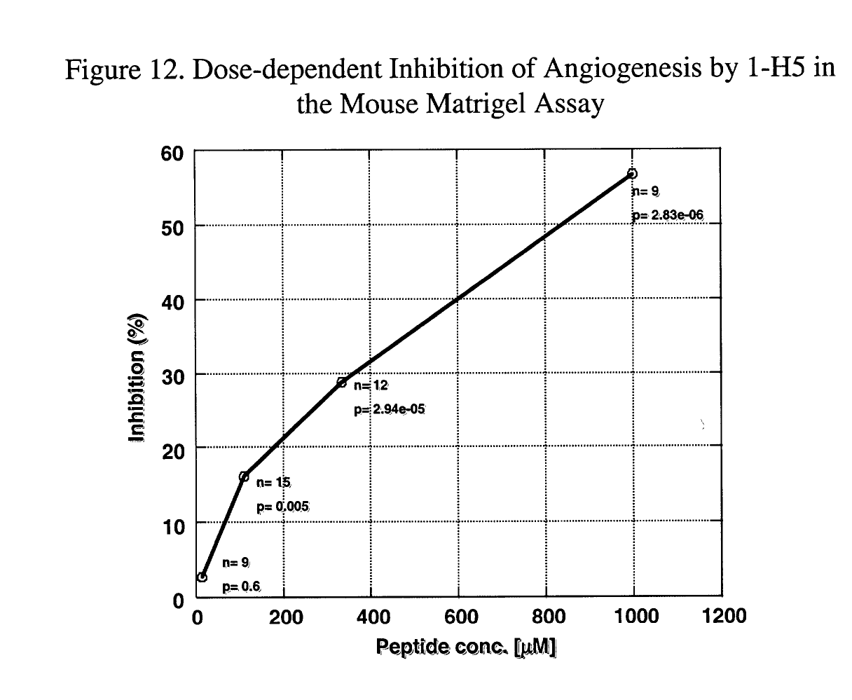

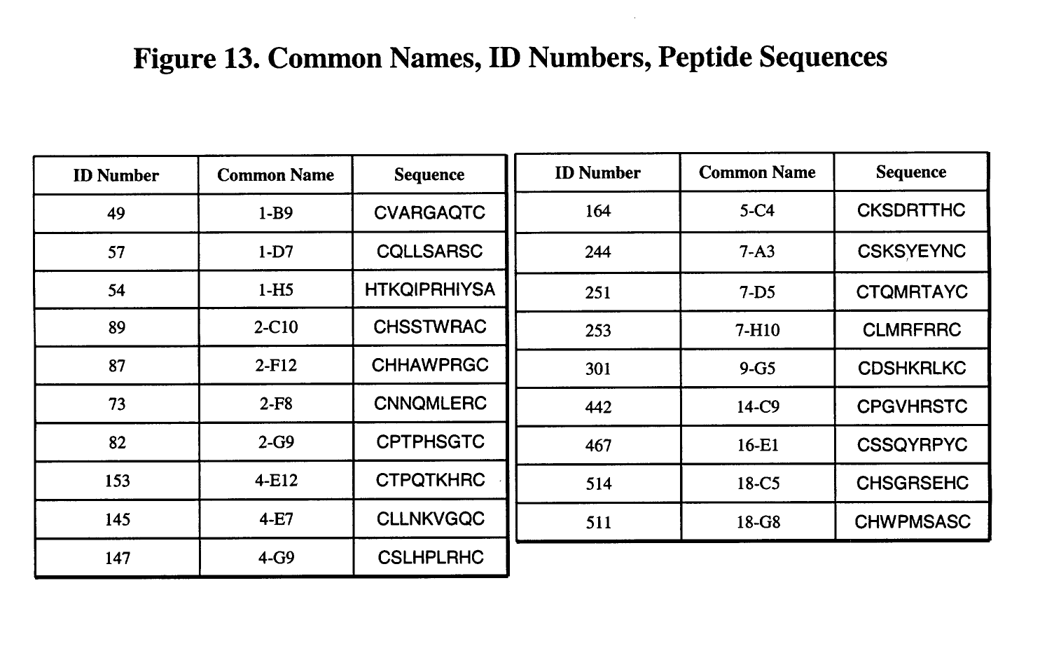

Inhibition of angiogenesis in the Matrigel model is also dose-dependent. FIG. 12 shows the result of one of the identified peptides (1-H5, SEQ ID NO: 54) in the mouse Matrigel plug assay. Inhibition of angiogenesis is linearly proportional to the concentration of 1-H5 in the Matrigel over a wide range of concentrations. All three points (concentrations) tested are statistically significant based on results from multiple experiments (n is no less than 9).

It should be noted that although the instant application provides data performed in two types of angiogenesis assays, other well-known angiogenesis assays can also be used with minor modification.

For example, the human saphenous vein, which runs from the groin to the ankle and is routinely harvested for coronary bypass surgery, is an excellent ex vivo model for angiogenesis. The vein is sectioned in 2-mm thick cross-sections and embedded into 12-well plates coated with Matrigel. The rings are cultured for 14 days before growth analysis. The limitations to this model are the amount of tissue available and donor-to-donor variation.

The mouse corneal angiogenesis assay offers a vascularization model in a normally avascular tissue. Pellets are inserted into surgically created micropockets in the cornea. This model has the distinct advantage of measuring only new vessel growth, which can be easily visualized. But because the cornea is normally avascular, it may not be an ideal testbed, as it lacks many of the blood-borne substances that modulate endothelial cell growth.

Finally, in the chick embryo chorioallantonic membrane (CAM) assay, a window is cut into the egg shell, exposing the vessels below. Alternatively, the embryo can be transferred to a petri dish, where the CAM grows as a flat membrane, thus allowing several test sites. This assay's potential caveats may include critical timing for observing effects, confounding inflammatory and vasodilation effects, and an inability to assess drugs requiring metabolic activation.

Equivalents

Those skilled in the art will recognize, or be able to ascertain using no more than routine experimentation, many equivalents to the specific embodiments of the invention described herein. Such equivalents are intended to be encompassed by the following claims.

All references, publications and patents cited in the specification above are herein incorporated by reference.

1. An isolated, synthetic or recombinant peptide or polypeptide which includes one or more ECBP sequences, each having an amino acid sequence independently represented in any of the following sequences:

| | |
|---|---|
| T-h-s-X-X-X-X-u-s-G-s-G-K or | (SEQ ID NO: 531) |
| h-p-X-X-Y-t-h-X-s-s or | (SEQ ID NO: 532) |
| S-X-X-u-M-s-V or | (SEQ ID NO: 533) |
| t-h-h-s-L-R-h-X-a-u or | (SEQ ID NO: 534) |
| s-s-G-h-X-s-X-a-a-h-p-s or | (SEQ ID NO: 535) |
| p-s-a-h-X-X-T-s-V-P-h or | (SEQ ID NO: 536) |
| L-X-N-p-s-p-p-t-G-t-t-t or | (SEQ ID NO: 537) |
| h-h-P-+-h-h-L-p-p-h-h-t or | (SEQ ID NO: 538) |
| s-s-l-h-s-h-s-s-X-p-p-A or | (SEQ ID NO: 539) |
| S-s-c-N-H-X-t-X-X-c-s or | (SEQ ID NO: 540) |
| s-t-h-H-X-X-X-t-X-h-s-L or | (SEQ ID NO: 541) |
| h-h-h-T-S-h-p-X-X-t-X-t-s-h or | (SEQ ID NO: 542) |
| h-X-h-X-S-h-s-h-p-L-p or | (SEQ ID NO: 543) |
| s-X-T-hX-p-X-H-s-oh-s or | (SEQ ID NO: 544) |
| t-o-s-T-S-h-h-s-s or | (SEQ ID NO: 545) |
| L-P-s-X-K-p-h-T-p-u-h-s or | (SEQ ID NO: 546) |
| h-S-P-t-Q-Q-p-hp-s-x-T-u or | (SEQ ID NO: 547) |
| t-W-K-s-P-S or | (SEQ ID NO: 548) |
| h-T-S-P-h-h or | (SEQ ID NO: 549) |
| u-h-p-S-t-h-T-h or | (SEQ ID NO: 550) |
| h-h-h-h-P-H-X-h-t-s or | (SEQ ID NO: 551) |
| A-X-X-p-X-X-o-p-P-h-h or | (SEQ ID NO: 552) |
| s-h-h-G-o-L-h-X-p-s-T-t-s-s or | (SEQ ID NO: 553) |
| h-p-X-H-h-X-X-p-S-o-X-t or | (SEQ ID NO: 554) |
| u-X-u-X-h-s-X-X-P-R-X-h or | (SEQ ID NO: 555) |
| h-h-X-R-P-X-X-M-P or | (SEQ ID NO: 556) |
| h-s-Q-T-s-T-X-h-X-h-h or | (SEQ ID NO: 557) |
| p-A-s-s-s-X-X-p-s-p-h-u or | (SEQ ID NO: 558) |
| s-S-h-t-M-K-P-S-P-p-P-L-s-A or | (SEQ ID NO: 559) |
| s-A-h-P-A-t-p-h-X-t-h-s-s or | (SEQ ID NO: 560) |
| T-p-s-Y-P-s-R-h-G-s-p-P-H-P-c or | (SEQ ID NO: 561) |
| s-X-P-s-h-G-P-h-A-a or | (SEQ ID NO: 562) |
| s-h-W-X-s-X-X-h-s or | (SEQ ID NO: 563) |
| s-T-G-s-s-s-P-X-S-t or | (SEQ ID NO: 564) |
| s-s-s or | (SEQ ID NO: 565) |
| s-T-X-H-s-s-p or | (SEQ ID NO: 566) |
| A-t-s-s-P-+-V-h-s-L-s or | (SEQ ID NO: 567) |
| s-.-h-P-P-t-t-L or | (SEQ ID NO: 568) |
| t-X-X-X-L-X-s-s-h-s-p-s or | (SEQ ID NO: 569) |
| L-P-X-X-X-h-L or | (SEQ ID NO: 570) |
| T-t-h-h-s-h-P-R-h-X-t or | (SEQ ID NO: 571) |
| L-X-X-s-X-X-X-O-p-q or | (SEQ ID NO: 572) |
| s-X-h-L-p-T-P-t-X-c-a-p-N or | (SEQ ID NO: 573) |
| h-t-h-X-s-P-P-X-h-X-X-s or | (SEQ ID NO: 574) |
| h-h-h-P-X-h-T-s-X-X-S-p or | (SEQ ID NO: 575) |
| t-s-s-X-Q-P-S-X-h-s-A-p or | (SEQ ID NO: 576) |
| E-t-X-s-L-X-R-h-L or | (SEQ ID NO: 577) |
| H-t-X-p-X-P-P-s-L-h-p-X-L or | (SEQ ID NO: 578) |
| p-h-s-X-s-l-l-P-X-p-h-X-F or | (SEQ ID NO: 579) |
| p-p-X-X-s-A-h-X-s-l-u-P-X-s or | (SEQ ID NO: 580) |
| H-h-s-X-S-P-p-h-s-W-s-X-L or | (SEQ ID NO: 581) |
| W-P-h-t-D-h-P or | (SEQ ID NO: 582) |
| s-p-T-t-A-P-l-s-X-s-s-h or | (SEQ ID NO: 583) |
| h-X-s-s-h-T-o-h-R-S or | (SEQ ID NO: 584) |
| t-h-X-h-s-o-t-X-A-P-A-t-P-h or | (SEQ ID NO: 585) |
| M-+-X-s-h-p-A-P-s-s-t-A-h-H or | (SEQ ID NO: 586) |
| u-p-t-p-p-X-X-h-T-h-s-s-h-X-s or | (SEQ ID NO: 587) |
| Y-Y-P-A-X-S-t-h-p-o-R-s or | (SEQ ID NO: 588) |
| t-T-t-T-h-L-a-u-X-t-p-t or | (SEQ ID NO: 589) |
| p-X-X-h-H-s-T-h-p-s-H-t-h or | (SEQ ID NO: 590) |
| s-N-X-X-X-X-T-X-s-t-p-s-X-h-p or | (SEQ ID NO: 591) |
| A-t-o-h-s-P-X-A-s-h-h or | (SEQ ID NO: 592) |
| h-H-s-X-p-h-N-X-X-s-T-X-s-+-s or | (SEQ ID NO: 593) |
| Y-q-h-X-s-X-p-s-X-p or | (SEQ ID NO: 594) |
| p-s-h-s-+-F-N-X-s-X-p-P or | (SEQ ID NO: 595) |
| s-p-h-s-X-h-s-P-u-X-h or | (SEQ ID NO: 596) |
| T-X-t-N-u-X-X-s-X-M-+-t or | (SEQ ID NO: 597) |
| S-M-V-Y-G-X-p-X-s-X-A or | (SEQ ID NO: 5981) |
| o-s-X-X-h-p-X-X-+-S-h-P-P-R-h or | (SEQ ID NO: 599) |
| t-h-t-P-h-S-X-S-h-X-h-P or | (SEQ ID NO: 600) |
| u-s-X-l-X-h-X-s-p-s or | (SEQ ID NO: 601) |
| h-t-Q-S-h-l-+-s-h-h-h-h or | (SEQ ID NO: 602) |
| s-p-p-X-h-s-L-t-S-s-p-X-h-h-h-D or | (SEQ ID NO: 603) |
| S-l-n-x-h-X-X-X-X-X-d-s or | (SEQ ID NO: 604) |
| S-L-H-X-L-X-t-D-h-h-h or | (SEQ ID NO: 605) |
| h-X-h-h-D-+-R-t-A-X-h-h or | (SEQ ID NO: 606) |
| p-X-u-X-X-X-X-R-X-s or | (SEQ ID NO: 607) |
| E-t-M-a-h-S-X-L or | (SEQ ID NO: 608) |
| N-X-X-X-X-p-p-h-h-t or | (SEQ ID NO: 609) |
| s-X-X-a-p-S-T-h-p-A-p-A or | (SEQ ID NO: 610) |
| S-p-X-h-X-t-Q-R-X-h-p-h or | (SEQ ID NO: 611) |
| p-X-h-X-Q-X-X-X-A-X-h-P or | (SEQ ID NO: 612) |
| u-s-X-p-h-t-X-S-S-h-t-h or | (SEQ ID NO: 613) |
| s-h-+-s-p-S-X-X-X-s-l-s-Y-p or | (SEQ ID NO: 614) |
| s-M-s-s-h-h-p-S-s-X-s-s-R or | (SEQ ID NO: 615) |
| h-h-s-h-Q-S-X-X-X-X-h or | (SEQ ID NO: 616) |
| o-X-h-s-S-M-h-h-h-s or | (SEQ ID NO: 617) |
| h-s-V-h-u-S-s-X-X-T or | (SEQ ID NO: 618) |
| h-S-t-L-P-H-h-X-L-s or | (SEQ ID NO: 619) |
| H-u-L-P-h-T-h-p-s-A-h or | (SEQ ID NO: 620) |
| h-h-X-p-p-T-H-X-h-P-h-p-s or | (SEQ ID NO: 621) |
| t-s-h-p-T-s-t-h-h-s-A or | (SEQ ID NO: 622) |
| t-s-T-s-Q-h-h-h-X-p-t-h or | (SEQ ID NO: 623) |
| h-X-h-s-p-D-V-p-h-X-h-h or | (SEQ ID NO: 624) |
| h-h-h-D-s-p-p-s-X-s-t-s-X-t or | (SEQ ID NO: 625) |
| p-X-X-X-X-X-R-h-T-X-h or | (SEQ ID NO: 626) |
| s-N-t-h-o-D-s-u-R-h or | (SEQ ID NO: 627) |
| I-X-X-X-c-L-T-X-P-s-P-t or | (SEQ ID NO: 628) |
| u-s-p-s-t-h-Q-s-R-t-h or | (SEQ ID NO: 629) |
| T-p-p-c-X-h-s-X-s-Y-h-A or | (SEQ ID NO: 630) |
| P-p-H-h-R-X-h-S-s-t-X-h or | (SEQ ID NO: 631) |
| s-p-+-c-h-p-X-u-R-t-h-p or | (SEQ ID NO: 632) |
| H-X-a-p-+-s-X-a-Y-p-s-A | (SEQ ID NO: 633) |

wherein
X represents any amino acid residue;
o represents an amino acid with an alcoholic side chain, e.g., Ser or Thr;
l represents an amino acid with an aliphatic side chain, e.g., lie or Leu or Val;
a represents an amino acid with an aromatic side chain, e.g., Phe or His or Trp or Tyr;
c represents an amino acid with a charged side chain, e.g., Glu or Asp or His or Lys or Arg;
h represents an amino acid with a hydrophobic side chain, e.g., Ala or Cys or Phe or Gly or His or Ile or Lys or Leu or Met or Arg or Thr or Val Try or Tyr;
− represents an amino acid with a negatively charged side chain, e.g., Glu or Asp;
p represents an amino acid with a polar side chain, e.g., Cys or Asp or Glu or His or Lys or Asn or Gin or Arg or Ser or Thr;
+ represents an amino acid with a positive side chain, e.g., His or Lys or Arg;
s represents an amino acid with a small side chain, e.g., Ala or Cys or Asp or Gly or Asn or Pro or Ser or Thr or Val;
u represents an amino acid with a tiny side chain, e.g., Ala or Gly or Ser; and
t represents an amino acid most likely situated at a turn, e.g., Ala or Cys or Asp or Glu or Gly or His or Lys or Asn or Gln or Arg or Ser or Thr.

2. An isolated or recombinant peptide or polypeptide which includes one or more ECBP sequence, each having an amino acid sequence independently represented in any of the following sequences:

| | |
|---|---|
| A-D-Y-R-S-SV-G-G-G-K or | (SEQ ID NO: 634) |
| L-S-N-N-s-K-H or | (SEQ ID NO: 635) |
| G-P-H-L-M-L-Q-N-K-L-R or | (SEQ ID NO: 636) |
| S-S-S-D-N-H-X-u-Q-L-H-T or | (SEQ ID NO: 637) |
| s-u-R-H-Q-S-W-H-P-H-D or | (SEQ ID NO: 638) |
| h-S-P-t-Q-Q-R-h-H-N-S-T or | (SEQ ID NO: 639) |
| A-P-I-H-L-H-S-c-P-L-L-H or | (SEQ ID NO: 640) |
| H-o-X-T-K-P-L or | (SEQ ID NO: 641) |
| H-s-I-Y-P-R-p or | (SEQ ID NO: 642) |
| Q-P-h-P-T-S-I or | (SEQ ID NO: 643) |
| h-A-s-u-S-M-P-T-s-R-L-A or | (SEQ ID NO: 644) |
| Y-H-h-P-P-S-s-T-P-L-s-A or | (SEQ ID NO: 645) |
| s-s-s-s-M-K-P-S-P-X-P or | (SEQ ID NO: 646) |
| T-T-s-Y-P-A-R-W-G-A-H-P or | (SEQ ID NO: 647) |
| L-P-I-s-K-A-L or | (SEQ ID NO: 648) |
| A-h-L-T-G-P-R or | (SEQ ID NO: 649) |
| p-S-L-H-Q-R-L or | (SEQ ID NO: 650) |
| H-Q-I-T-Q-P-p-S-L-L-S-P or | (SEQ ID NO: 651) |
| A-I-P-X-V-P or | (SEQ ID NO: 652) |
| H-K-A-P-S-P-K-h-D-W-s-P or | (SEQ ID NO: 653) |
| E-T-p-A-P-L or | (SEQ ID NO: 654) |
| G-E-T-X-A-P-h or | (SEQ ID NO: 655) |
| M-K-S-s-I-P-A-P-s-G-G or | (SEQ ID NO: 656) |
| S-P-F-R-A-P-s or | (SEQ ID NO: 657) |
| Y-P-h-R-A-P-T-s-Q-A-h-H or | (SEQ ID NO: 658) |
| S-T-A-o-Y-T-R or | (SEQ ID NO: 659) |
| Y-Y-P-A-u-S-T-I-Q-S-R-P or | (SEQ ID NO: 660) |
| H-D-T-Y-s-s-H or | (SEQ ID NO: 661) |
| H-A-A-T-M-P or | (SEQ ID NO: 662) |
| S-R-F-N-X-D or | (SEQ ID NO: 663) |
| T-X-p-N-G-P-S or | (SEQ ID NO: 664) |
| G-X-T-P-S-h-A or | (SEQ ID NO: 665) |
| S-M-V-Y-G-N-p-L-P-S-A-L or | (SEQ ID NO: 666) |
| h-A-h-S-M-P-P or | (SEQ ID NO: 667) |
| T-E-Q-p-W-I-K-N-I-Y-A-R or | (SEQ ID NO: 668) |
| A-L-H-S-A-R or | (SEQ ID NO: 669) |
| h-L-H-S-D-R-A-L-M-I-D or | (SEQ m NO: 670) |
| S-A-P-L-t-S or | (SEQ ID NO: 671) |
| H-S-S-T-h-R-A or | (SEQ ID NO: 672) |
| S-p-P-W-s-A-Q-R-E-L-S-V or | (SEQ ID NO: 673) |
| u-T-W-S-H-H-h-S-S-u-u-L or | (SEQ ID NO: 674) |
| G-W-S-S-Y-R or | (SEQ ID NO: 675) |
| A-M-s-P-R-p-H-S-s-P-S-V or | (SEQ ID NO: 676) |
| M-P-A-V-M-S-S-s-Q-V-P-R or | (SEQ ID NO: 677) |
| L-L-A-D-T-T-H-H-h-P-W-T or | (SEQ ID NO: 678) |
| K-N-L-N-T-T-u-M-Y-A-A-S or | (SEQ ID NO: 679) |
| I-L-A-X-D-L-T-X-X-G-P or | (SEQ ID NO: 680) |
| O-G-K-W-Q-P-R or | (SEQ ID NO: 681) |
| G-L-Q-u-R-H-I or | (SEQ ID NO: 682) |
| K-h-I-P-t-T-Y or | (SEQ ID NO: 683) |
| Q-S-H-Y-R-X-I-S-P-A-Q-V | (SEQ ID NO: 684) |

wherein
X represents any amino acid residue;
o represents an amino acid with an alcoholic side chain, e.g., Ser or Thr;
l represents an amino acid with an aliphatic side chain, e.g., Ile or Leu or Val;
a represents an amino acid with an aromatic side chain, e.g., Phe or His or Trp or Tyr;
c represents an amino acid with a charged side chain, e.g., Glu or Asp or His or Lys or Arg;
h represents an amino acid with a hydrophobic side chain, e.g., Ala or Cys or Phe or Gly or His or Ile or Lys or Leu or Met or Arg or Thr or Val Try or Tyr;
− represents an amino acid with a negatively charged side chain, e.g., Glu or Asp;
p represents an amino acid with a polar side chain, e.g., Cys or Asp or Glu or His or Lys or Asn or Gln or Arg or Ser or Thr;
+ represents an amino acid with a positive side chain, e.g., His or Lys or Arg;
s represents an amino acid with a small side chain, e.g., Ala or Cys or Asp or Gly or Asn or Pro or Ser or Thr or Val;
u represents an amino acid with a tiny side chain, e.g., Ala or Gly or Ser; and
t represents an amino acid most likely situated at a turn, e.g., Ala or Cys or Asp or Glu or Gly or His or Lys or Asn or Gln or Arg or Ser or Thr.

3. An isolated, synthetic or recombinant peptide or polypeptide which includes one or more ECBP sequences, each having an amino acid sequence independently represented in any of SEQ ID Nos: 1-530.

4. A peptidomimetic comprising a binding sequence corresponding to an ECBP sequence represented in any of claims 1-3, having one or more peptide bond replacements or non-naturally occurring amino acid sidechains, wherein the peptidomimetic binds to a endothelial cell in a manner dependent upon the presence of the ECBP sequence.

5. The peptide/polypeptide of claim 3, wherein the ECBP sequence mediates binding to endothelial cells with a K_{d} of 10⁻⁵ or less.

6. The peptide/polypeptide of claim 3, which has an EC₅₀ of 10⁻⁴ M or less for promoting at least one of endothelial cell proliferation or endothelial cell migration.

7. The peptide/polypeptide of claim 3, which has an ED₅₀ of 10⁻⁴ M or less for inhibiting at least one of endothelial cell proliferation or endothelial cell migration.

8. The peptide/polypeptide of claim 7, which has an ED₅₀ for killing endothelial cells at least one order of magnitude greater than the ED₅₀ for inhibiting endothelial cell proliferation or endothelial cell migration.

9. The peptide/polypeptide of claim 3, covalently or non-covalently coupled to a cytotoxic agent or antiproliferative agent.

10. The peptide/polypeptide of claim 7, wherein the agent is selected from: alkylating agents, enzyme inhibitors, proliferation inhibitors, lytic agents, DNA or RNA synthesis inhibitors, membrane permeability modifiers, DNA intercalators, metabolites, dichloroethylsulfide derivatives, protein production inhibitors, ribosome inhibitors, inducers of apoptosis, or neurotoxins.

11. The peptide/polypeptide of claim 7, coupled to a cytotoxic agents selected from: taxanes; nitrogen mustards; ethylenimine derivatives; alkyl sulfonates; nitrosoureas; triazenes; folic acid analogs; pyrimidine analogs; purine analogs; vinca alkaloids; antibiotics; enzymes; platinum coordination complexes; substituted urea; methyl hydrazine derivatives; adrenocortical suppressants; or hormones and antagonists.

12. The peptide/polypeptide of claim 7, coupled to a protein synthesis inhibitor.

13. The peptide/polypeptide of claim 7, coupled to a toxin selected from: ricin toxin, Pseudomonas exotoxin (PE), diphtheria toxin (DT), Clostridium perfringens phospholipase C (PLC), bovine pancreatic ribonuclease (BPR), pokeweed antiviral protein (PAP), abrin, abrin A chain (abrin toxin), cobra venom factor (CVF), gelonin (GEL), saporin (SAP), modeccin, viscumin or volkensin.

14. The peptide/polypeptide of claim 7, coupled to an enzyme which converts a prodrug to an active drug.

15. The peptide/polypeptide of claim 7, coupled with an agent selected from: metals; metal chelators; lanthanides; lanthanide chelators; radiometals; radiometal chelators; positron-emitting nuclei; microbubbles (for ultrasound); liposomes; molecules microencapsulated in liposomes or nanosphere; monocrystalline iron oxide nanocompounds; magnetic resonance imaging contrast agents; light absorbing, reflecting and/or scattering agents; colloidal particles; or fluorophores.

16. The peptide/polypeptide of claim 15, coupled to a metal-chelating ligand.

17. The peptide/polypeptide of claim 16, wherein the metal-chelating ligand is an NₓS_{y}chelate moiety.

18. The peptide/polypeptide of claim 17, wherein the metal-chelating ligand chelates a radiometal or paramagnetic ion.

19. An imaging preparation comprising the peptide/polypeptide of claim 16, including a chelated metal selected from: ³²P, ³³P, ⁴³K, ⁴⁷Sc, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/^{81M}Kr ^{87M}Sr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb ¹²¹Sn, 123I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³ Pb, ²¹¹At, ²¹²Pb, ²¹²Bi and ²¹³Bi.

20. The peptide/polypeptide of claim 7, coupled with a radiosensitizing agent.

21. The peptide/polypeptide of claim 3, coupled to a polymer or a functionalized polymer.

22. The peptide of claim 3, which peptide or peptidomimetic is cyclic.

23. The peptide of claim 22, having a cyclic portion formed by one or more intramolecular covalent bonds between two or more amino acid residues of the peptide.

24. The peptide of claim 23, which intramolecular bond is selected from: backbone-to-backbone, sidechain-to-backbone or sidechain-to-sidechain bonds.

25. The peptide of claim 23, wherein said intramolecular bond is an intramolecular disulfide bond.

26. The peptide of claim 23, wherein said intramolecular bond selected from: backbone-to-backbone, sidechain-to-backbone or sidechain-to-sidechain bond.

27. The peptide of claim 3, formulated with a polyanionic or polycatonic binding agent.

28. The peptide/polypeptide of claim 3, formulated in a pharmaceutically acceptable excipient.

29. The peptide/polypeptide of claim 3, which polypeptide is a chimeric serum protein.

30. The peptide/polypeptide of claim 29, which ECBP sequence(s) are present at one or more internal sites in the amino acid sequence of the serum protein.

31. The peptide/polypeptide of claim 29, which serum protein is selected from: albumin, α-globulins, β-globulins, γ-globulins, haptoglobin, transthyretin, collagen, α2 macroglobulin, β2 microglobulin, C Reactive Protein, apolipoproteins, lipoproteins, cathepsins amylase, antichymotrypsin, ferritin, α fetoprotein, elastin and fibronectin and coagulation factors including fibrinogen, fibrin, thrombin, ceruloplasmin, antiplasmin or antithrombin III.

32. A chimeric viral coat protein comprising a peptide/polypeptide of claim 3 fused to a viral coat protein.

33. A nucleic acid including a coding sequence for the peptide/polypeptide of claim 3.

34. A nucleic acid encoding the peptide/polypeptide of claim 29.

35. A nucleic acid encoding the peptide/polypeptide of claim 32.

36. The nucleic acid of claim 34, wherein said coding sequence is flanked at each end by a coding sequence of an intein polypeptide to encode a fusion protein which, when expressed, undergoes intramolecular splicing to yield a cyclic peptide including said one or more ECBP sequences.

37. A viral particle including one or more chimeric viral coat proteins of claim 32.

38. The viral particle of claim 37, which is an adenoviral particle or an adeno-associated viral particle.

39. The viral particle of claim 38, wherein the ECBP sequence is provided as part of a fusion protein including the fiber knob protein.

40. A method for promoting the proliferation and/or migration of endothelial cells comprising treating the cells with an ECBP agonist in an amount sufficient to promote proliferation and/or migration of the treated cells.

41. A method for reducing the proliferation and/or migration of endothelial cells comprising treating the cells with an ECBP antagonist in an amount sufficient to reduce proliferation and/or migration of the treated cells.

42. A method for reducing angiogenesis comprising treating an mammal with an ECBP antagonist in an amount sufficient to reduce angiogenesis at one or more sites in the treated mammal.

43. The method of claim 42, for prophylaxis or reducing the effects of a disorder selected from: hemangioma, solid tumors, leukemia, metastasis, telangiectasia, psoriasis, scleroderma, pyogenic granuloma, myocardial angiogenesis, plaque neovascularization, coronary collaterals, cerebral collaterals, arteriovenous malformations, ischemic limb angiogenesis, corneal diseases, rubeosis, neovascular glaucoma, diabetic retinopathy, retrolental fibroplasia, arthritis, diabetic neovascularization, macular degeneration, wound healing, peptic ulcer, Helicobacter related diseases, fractures, keloids, vasculogenesis, hematopoiesis, ovulation, menstruation, placentation, or cat scratch fever.

44. The method of claim 42, as part of a treatment regimen for treatment of a solid tumor or carcinoma.

45. The method of claim 42, as part of a treatment regimen for treatment or prophylaxis for an autoimmune disease.

46. The method of claim 42, as part of a treatment regimen for treatment or prophylaxis for an ocular diseases selected from: diabetic retinopathy, retinopathy of prematurity, corneal graft rejection, retrolental fibroplasia, neovascular glaucoma, rubeosis, retinal neovascularization due to macular degeneration or hypoxia.

47. The method of claim 42, as part of a treatment regimen for treatment or prophylaxis for psoriasis.

48. The method of claim 42, in combination with radiotherapy and/or other chemotherapeutic treatments.

49. The method of any of claims 40-48, wherein the ECBP antagonist is a peptide/ polypeptide of claim 6.

50. A method for promoting angiogenesis comprising treating an mammal with an ECBP agonist in an amount sufficient to promote angiogenesis at one or more sites in the treated mammal.

51. The method of claim 50, as part of a treatment regimen for myocardial infarction.

52. The method of claim 50, as part of a treatment regimen for repair of vascular damage after ischemia.

53. The method of claim 50, as part of a treatment regimen to stimulate the growth of transplanted tissue or vascularized prosthetic devices.

54. The method of claim 50, as part of a treatment regimen to stimulate wound healing.

55. The method of claim 50, as part of a treatment regimen for vascular tissue repair during or after angioplasty.

56. The method of any of claims 50-55, wherein the ECBP agonist is a peptide/polypeptide of claim 6.

57. The method of any of claims 40-55, wherein the ECBP agonist or ECBP antagonist is delivered systemically.

58. The method of any of claims 40-55, wherein the ECBP agonist or ECBP antagonist is delivered by local injection.

59. A medicament for promoting angiogenesis comprising an ECBP agonist in an amount sufficient to promote angiogenesis at one or more sites in the treated mammal.

60. A method for manufacturing a medicament for promoting angiogenesis comprising admixing an ECBP agonist, in an amount sufficient to promote angiogenesis at one or more sites in a treated mammal, with a pharmaceutically acceptable excipient.

61. A medicament for reducing angiogenesis comprising an ECBP antagonist in an amount sufficient to reduce angiogenesis at one or more sites in the treated mammal.

62. A method for manufacturing a medicament for inhibiting angiogenesis comprising admixing an ECBP antagonist, in an amount sufficient to reduce angiogenesis at one or more sites in a treated mammal, with a pharmaceutically acceptable excipient.

63. A method of conducting a drug discovery business comprising:
(i) identifying, from a variegated library of peptides, members of the library bind to endothelial cells;
(ii) from the members of the library identified in step (i), identifying peptides which inhibit or promote growth and/or migration of endothelial cells;
(iii) conducting therapeutic profiling of an agent including the peptide identified in step (ii), or peptidomimetic thereof or a protein containing the peptide for efficacy and toxicity in mammals; and
(iv) formulating a pharmaceutical preparation including one or more agents identified in step (iii) as having an acceptable therapeutic profile.

64. The method of claim 63, including an additional step of establishing a distribution system for distributing the pharmaceutical preparation for sale, and may optionally include establishing a sales group for marketing the pharmaceutical preparation.

65. A method of conducting a drug discovery business comprising:
(i) identifying, from a variegated library of peptides, members of the library bind to endothelial cells;
(ii) from the members of the library identified in step (i), identifying peptides which inhibit or promote growth and/or migration of endothelial cells;
(iii) conducting therapeutic profiling of an agent including the peptide identified in step (ii), or peptidomimetic thereof or a protein containing the peptide for efficacy and toxicity in mammals; and
(iv) licensing, to a third party, the rights for further drug development of one or more agents identified in step (iii) as having an acceptable therapeutic profile.

66. A method of conducting a drug discovery business comprising:
(i) identifying, from a variegated library of peptides, members of the library bind to endothelial cells;
(ii) from the members of the library identified in step (i), identifying peptides which inhibit or promote growth and/or migration of endothelial cells;
(iii) licensing, to a third party, the rights for further drug development based on one or more peptides identified in step (ii).
